(19) United States
(12) Patent Application Publication
(10) Pub. No.: US 2023/0234967 A1
(43) Pub. Date: Jul. 27, 2023

(54) Fused Azole Heterocycles as AHR Antagonists

(71) Applicant: Senda Biosciences, Inc., Cambridge, MA (US)

(72) Inventors: Alessandra Bartolozzi, Gladwyne, PA (US); John Robert Proudfoot, Newtown, CT (US); Timothy Briggs, Waltham, MA (US); John Mancuso, Saint-Marthe-Sur-Le-Lac, Québec (CA); Maxence Bos, Montreal (CA); Tianlin Guo, Montreal (CA); Vu Linh Ly, Montreal (CA); Anna Blois, Chestnut Hill, MA (US); Bernard Lanter, Somerville, MA (US); Steven John Taylor, Winchester, MA (US); Leonard Buckbinder, East Greenwich, RI (US)

(21) Appl. No.: 17/999,443

(22) PCT Filed: May 27, 2021
(86) PCT No.: PCT/US2021/034413

§ 371 Date: Nov. 21, 2022

(51) Int. Cl. C07D 513/04; C07D 498/04; C07D 471/04; C07D 487/04; A61P 35/00; A61P 1/00; A61K 39/395; A61K 31/437; A61K 31/444; A61K 31/5377; A61K 31/496; A61K 31/519; A61K 31/4985

(52) U.S. Cl. CPC C07D 513/04; C07D 498/04; C07D 471/04; C07D 487/04; A61P 35/00; A61P 1/00; A61K 39/3955; A61K 31/437; A61K 31/444; A61K 31/5377; A61K 31/496; A61K 31/519; A61K 31/4985

(57) ABSTRACT

The present disclosure relates to thiazolo-pyridine, oxazolo-pyridine, pyrrolo-pyridine, pyrrolo-pyrazine and pyrrolo-pyrimidine compounds and pharmaceutically acceptable salts thereof, pharmaceutical compositions comprising the same, methods of preparing the same, intermediate compounds useful for preparing the same, and methods for treating or prophylaxis of diseases, in particular cancer or conditions with dysregulated immune responses or other disorders associated with aberrant AHR signaling.

This application claims the benefit of priority to U.S. Provisional Patent Application No. 63/031,391, filed May 28, 2020, which is incorporated herein by reference in its entirety.

Disclosed herein are novel thiazolo-pyridine, oxazolo-pyridine, pyrrolo-pyridine, pyrrolo-pyrazine and pyrrolo-pyrimidine compounds and pharmaceutically acceptable salts thereof, methods of preparing said compounds and salts, intermediate compounds useful for preparing said compounds and salts, pharmaceutical compositions comprising said compounds and salts, and methods of using said compounds and salts for the treatment or prophylaxis of diseases, in particular of cancer or conditions with dysregulated immune responses or other disorders associated with aberrant AHR signaling.

The Aryl Hydrocarbon Receptor (AHR) is a ligand-activated transcription factor, belonging to the basic helix-loop-helix/Per-Arnt-Sim (bHLH/PAS) family that is located in the cytosol. Upon ligand binding, the AHR translocates to the nucleus where it heterodimerises with ARNT (AHR Nuclear Translocator) upon which it interacts with DREs (Dioxin Response Elements) of AHR-responsive genes to regulate their transcription. The AHR is best known for binding to environmental toxins and inducing the metabolic machinery, such as cytochrome P 450 enzymes (eg. CYP1A1, CYP1A2 and CYP1B1), required for their elimination (Reyes et al., Science, 1992, 256(5060):1 193-5). Activation of AHR by xenobiotics has demonstrated its role in numerous cellular processes such as embryogenesis, tumorigenesis and inflammation.

AHR is expressed in many cells of the immune system, including dendritic cells (DCs), macrophages, T cells and NK cells, and plays an important role in immunoregulation (Nguyen et al., Front. Immunol., 2014, 5:551). The classic exogenous AHR ligands TCDD and 3-methylcholanthrene, for example, are known to induce profound immunosuppression, promote carcinogenesis and induce tumour growth (Gramatzki et al., Oncogene, 2009, 28(28):2593-605; Bui et al., Oncogene, 2009, 28(41):3642-51; Esser et al., Trends Immunol., 2009, 30:447-454). In the context of immunosuppression, AHR activation promotes regulatory T cell generation, inhibits Th1 and Th17 differentiation, directly and indirectly, and decreases the activation and maturation of DCs (Wang et al., Clin. Exp. Immunol., 2014, 177(2):521-30; Mezrich et al., J. Immunol., 2010, 185(6):3190-8; Wei et al., Lab. Invest., 2014, 94(5):528-35; Nguyen et al., PNAS, 2010, 107(46):19961-6). AHR activation modulates the innate immune response and constitutive AHR expression has been shown to negatively regulate the type-1 interferon response to viral infection (Yamada et al., Nat. Immunol., 2016, 17(6):687-94). Additionally, mice with a constitutively active AHR spontaneously develop tumours (Andersson et al., PNAS, 2002, 99(15):9990-5).

In addition to xenobiotics, the AHR can also bind metabolic products of tryptophan degradation. Tryptophan metabolites, such as kynurenine and kynurenic acid, are endogenous AHR ligands that activate the AHR under physiological conditions (DiNatale et al., Toxicol. Sci., 2010, 115(1):89-97; Mezrich et al., J. Immunol., 2010, 185(6):3190-8; Opitz et al., Nature, 2011, 478(7368):197-203). Other endogenous ligands are known to bind the AHR, although their physiological roles are currently unknown (Nguyen & Bradfield, Chem. Res. Toxicol., 2008, 21(1):102-116).

The immunosuppressive properties of kynurenine and tryptophan degradation are well described and are implicated in cancer-associated immunosuppression. The enzymes indoleamine-2,3-dioxygenases 1 and 2 (IDO1/IDO2) as well as tryptophan-2,3-dioxygenase 2 (TDO2) are responsible for catalysing the first and rate-limiting step of tryptophan metabolism. IDO1/2-mediated degradation of tryptophan in tumours and tumour-draining lymph nodes reduces anti-tumour immune responses and inhibition of IDO can suppress tumour formation in animal models (Uyttenhove et al., Nat. Med., 2003, 9(10):1269-74; Liu et al., Blood, 2005, 115(17): 3520-30; Muller et al., Nat. Med., 11(3):312-9; Metz, Cancer Res., 2007, 67(15):7082-7).

TDO2 is also strongly expressed in cancer and can lead to the production of Immunosuppressive kynurenine. In glioma, activation of the AHR by kynurenine, downstream of TDO-mediated tryptophan degradation, enhances tumour growth as a consequence of inhibiting anti-tumour immune responses as well as directly promoting tumour cell survival and motility (Opitz et al., Nature, 2011, 478(7368):197-203). AHR ligands generated by tumour cells therefore act in both an autocrine and paracrine fashion on tumour cells and lymphocytes, respectively, to promote tumour growth.

The present disclosure is drawn to novel thiazolo-pyridine, oxazolo-pyridine, pyrrolo-pyridine, pyrrolo-pyrazine and pyrrolo-pyrimidine compounds and/or pharmaceutically acceptable salts thereof. Compounds of the present disclosure have surprisingly been found to effectively inhibit AHR and may therefore be used for treatment or prophylaxis of cancer and/or other conditions where exogenous and endogenous AHR ligands induce dysregulated immune responses, uncontrolled cell growth, proliferation and/or survival of tumor cells, immunosuppression in the context of cancer, inappropriate cellular immune responses, or inappropriate cellular inflammatory responses or diseases that are accompanied by uncontrolled cell growth, proliferation and/or survival of tumor cells, immunosuppression in the context of cancer inappropriate cellular immune responses, or inappropriate cellular inflammatory responses, particularly in which the uncontrolled cell growth, proliferation and/or survival of tumor cells, immunosuppression in the context of cancer, inappropriate cellular immune responses, or inappropriate cellular inflammatory responses is mediated by AHR, such as, for example, liquid and solid tumors, and/or metastases thereof, e.g. head and neck tumors including brain tumors and brain metastases, tumors of the thorax including non-small cell and small cell lung tumors, gastrointestinal tumors including colon, colorectal and pancreatic tumors, liver tumors, endocrine tumors, mammary and other gynecological tumors, urological tumors including renal, bladder and prostate tumors, skin tumors, and sarcomas, and/or metastases thereof.

The present disclosure also relates to pharmaceutical compositions comprising at least one entity chosen from the thiazolo-pyridine, oxazolo-pyridine, pyrrolo-pyridine, pyrrolo-pyrazine and pyrrolo-pyrimidine compounds disclosed herein and pharmaceutically acceptable salts thereof. The present disclosure also relates to methods of treatment comprising administering at least one compound, pharmaceutically acceptable salt thereof, and/or pharmaceutical composition of the present disclosure. In some embodiments, the disclosure provides a method of treating a disease or condition mediated by AHR signaling. In some embodiments, the disclosure provides a method of treating a disease or condition associated with aberrant AHR signaling. In some embodiments, the disclosure provides a method of inhibiting cancer cell proliferation mediated by AHR signaling.

As used herein, the term “pharmaceutically acceptable salt” refers to a salt that is pharmaceutically acceptable as defined herein and that has the desired pharmacological activity of the parent compound. Non-limiting examples of pharmaceutically acceptable salts include those derived from inorganic acids, non-limiting examples of which include hydrochloric acid, hydrobromic acid, hydroiodic acid, sulfuric acid, nitric acid, and phosphoric acid, and those derived from organic acids, non-limiting examples of which include acetic acid, trifluoroacetic acid, propionic acid, glycolic acid, pyruvic acid, oxalic acid, stearic acid, malic acid, maleic acid, malonic acid, salicylic acid, succinic acid, fumaric acid, tartaric acid, citric acid, benzoic acid, cinnamic acid, mandelic acid, p-toluenesulfonic acid, methanesulfonic acid, ethanesulfonic acid, and lactic acid.

Additional non-limiting examples of pharmaceutically acceptable salts include those formed when an acidic proton in a parent compound is replaced by a metal ion, non-limiting examples of which include an alkali metal ion and an alkaline earth metal ion, and those formed when an acidic proton present in a parent compound is replaced by a ammonium ion, a primary ammonium ion, a secondary ammonium ion, a tertiary ammonium ion, or a quaternary ammonium ion. Non-limiting examples of alkali metals and alkaline earth metals include sodium, potassium, lithium, calcium, aluminum, magnesium, copper, zinc, iron, and manganese. Additional non-limiting examples of pharmaceutically acceptable salts include those comprising one or more counterions and zwitterions.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. The same rule applies for any other ranges described herein, even if the values within the range are not specifically called out in this disclosure.

The term “compound,” as used herein unless otherwise indicated, refers to a collection of molecules having an identical chemical structure as a collection of stereoisomers (for example, a collection of racemates, a collection of cis/trans stereoisomers, or a collection of (F) and (Z) stereoisomers). Therefore, geometric and conformational mixtures of the present compounds and salts are within the scope of the disclosure. Unless otherwise stated, all tautomeric forms of the compounds of the disclosure are within the scope of the disclosure.

“Stereoisomer” as used herein refers to enantiomers and diastereomers.

The term “tautomer,” as used herein, refers to one of two or more isomers of a compound that exist together in equilibrium, and are readily interchanged by migration of an atom or group within the molecule.

Unless indicated otherwise, nomenclature used to describe chemical groups or moieties as used herein follow the convention where, reading the name from left to right, the point of attachment to the rest of the molecule is at the right-hand side of the name. For example, the group “(C₁₋₃ alkoxy)C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, where a chemical group is described by its chemical formula or structure having a terminal bond moiety indicated by “−”, it will be understood that the “−” represents the point of attachment. In some embodiments, a wavy line (i.e., [symbol] ) depicts the point of attachment.

As used herein, an “acyl” or “alkanoyl” is a functional group with formula RCO— where R is bound to the carbon atom of the carbonyl functional group by a single bond and the “—” denotes the point of attachment to the rest of the molecule. Non-limiting examples of acyls include formyl (HC(O)—, also called methanoyl), acetyl (CH₃C(O)—, also called ethanoyl), and benzoyl (PhC(O)—).

The term “alkyl” or “aliphatic” as used herein, means a straight-chain (i.e., unbranched) or branched, substituted or unsubstituted hydrocarbon chain that is completely saturated and that has a single point of attachment to the rest of the molecule. Unless otherwise specified, an alkyl group is a hydrocarbon chain of 1 to 20 alkyl carbon atoms. In some embodiments, an alkyl group contains one to twelve carbon atoms (C₁-C₁₂). In some embodiments, an alkyl group contains one to eight carbon atoms (C₁-C₈). In some embodiments, an alkyl group contains one to six carbon atoms (C₁-C₆). In some embodiments, an alkyl group contains one to four carbon atoms (C₁-C₄). In some embodiments, a cyclic alkyl group contains three to six carbon atoms (C₃-C₆). Non-limiting examples of substituted and unsubstituted linear, branched, and cyclic alkyl groups include methyl, ethyl, n-propyl, iso-propyl, cyclopropyl, n-butyl, sec-butyl, iso-butyl, tert-butyl, cyclobutyl, cyclopentyl, cyclohexyl, hydroxymethyl, chloromethyl, fluoromethyl, trifluoromethyl, aminomethyl, 2-aminoethyl, 3-aminopropyl, 4-aminobutyl, dimethylaminomethyl, 2-dimethylaminoethyl, 3-dimethylaminopropyl, 4-dimethylaminobutyl, cyclopropylmethyl, cyclobutylmethyl, cyclopentylmethyl, trifluoroethyl, and trifluoropropyl.

“Alkoxy,” as used herein, refers to an alkyl group, as previously defined, attached to the principal carbon chain through an oxygen (“alkoxy”) atom.

“Halo” and “halogen,” as used herein, are interchangeable and refer to halogen atoms such as fluoro (F), chloro (Cl), bromo (Br), and iodo (I).

“Haloalkyl” refers to an alkyl group substituted with one or more halo atoms (F, Cl, Br, I). For example, “fluoromethyl” refers to a methyl group substituted with one or more fluoro atoms (e.g., monofluoromethyl, difluoromethyl, or trifluoromethyl).

“Haloalkoxy” refers to an alkoxy group substituted with one or more halo atoms (F, Cl, Br, I). For example, “fluoromethoxy” refers to a methoxy group substituted with one or more fluoro atoms (e.g., monofluoromethoxy, difluoromethoxy, or trifluoromethoxy).

“Hydroxyalkyl” refers to an alkyl group substituted with one or more hydroxy groups (—OH).

The terms “cycloalkyl” and “cycloalkyl group” as used interchangeably herein refer to a cyclic saturated monovalent hydrocarbon radical of three to twelve carbon atoms that has a single point of attachment to the rest of the molecule. Cycloalkyl groups may be unsubstituted or substituted. In some embodiments, a cycloalkyl group comprises three to eight carbon atoms (C₃-C₈). In some embodiments, a cycloalkyl group comprises three to six carbon atoms (C₃-C₆). Non-limiting examples of substituted and unsubstituted cycloalkyls include cyclopropyl, cyclopropylmethyl, cyclobutyl, cyclobutylmethyl, cyclopentyl, cyclopentylmethyl, cyclohexyl, cycloheptyl, and cyclooctyl.

The terms “alkylene” and “alkylene group” as used interchangeably herein refer to a saturated divalent (i.e., having two points of attachment to the rest of the molecule) hydrocarbon radical comprising one to twelve carbon atoms (C₁-C₁₂). Alkylene groups may be linear, branched, or cyclic. Alkylene groups may be unsubstituted or substituted. In some embodiments, an alkylene group comprises one to eight carbon atoms (C₁-C₈). In some embodiments, an alkylene group comprises one to six carbon atoms (C₁-C₆). In some embodiments, an alkylene group comprises one to four carbon atoms (C₁-C₄). Non-limiting examples of alkylene groups include methylene and ethylene.

The terms “alkenyl” and “alkenyl group” as used interchangeably herein refer to a monovalent (i.e., having a single point of attachment to the rest of the molecule) hydrocarbon radical comprising two to eight carbon atoms (C₂-C₈) with at least one site of unsaturation (i.e., an sp2 carbon-carbon double bond). Alkenyl groups may be linear, branched, or cyclic. Alkenyl groups may be unsubstituted or substituted. In some embodiments, an alkenyl group contains two to six carbon atoms (C₂-C₆). In some embodiments, an alkenyl group contains two to four carbon atoms (C₂-C₄). Alkenyl groups may have E or Z orientations. Non-limiting examples of alkenyl groups include ethenyl (also called vinyl), 1-propenyl, iso-propenyl, and 2-chloroethenyl.

The terms “alkenylene” and “alkenylene group” as used interchangeably herein refer to a divalent (i.e., having two points of attachment to the rest of the molecule) hydrocarbon radical of two to eight carbon atoms (C₂-C₈) with at least one site of unsaturation (e.g., an sp2 carbon-carbon double bond). Alkenylene groups may be linear, branched, or cyclic. Alkenylene groups may be unsubstituted or substituted. In some embodiments, an alkylene group contains two to six carbon atoms (C₂-C₆). In some embodiments, an alkylene group contains two to four carbon atoms (C₂-C₄). Alkylene groups may have E or Z orientations. A non-limiting example of an alkenyl group is ethenylene (also called vinylene).

The terms “alkynyl” and “alkynyl group” as used interchangeably herein refer to a monovalent (i.e., having a single point of attachment to the rest of the molecule) hydrocarbon radical of two to eight carbon atoms (C₂-C₈) with at least one site of unsaturation (i.e., an sp carbon-carbon triple bond). Alkynyl groups may be linear or branched. Alkynyl groups may be unsubstituted or substituted. In some embodiments, an alkynyl group contains two to six carbon atoms (C₂-C₆). In some embodiments, an alkynyl group contains two to four carbon atoms (C₂-C₄). A non-limiting example of an alkynyl group is ethynyl.

The terms “alkynylene” and “alkynylene group” as used interchangeably herein refer to a divalent (i.e., having two points of attachment to the rest of the molecule) hydrocarbon radical of two to eight carbon atoms (C₂-C₈) with at least one site of unsaturation (i.e., an sp carbon-carbon triple bond). Alkynylene groups may be linear or branched. Alkynylene groups may be unsubstituted or substituted. In some embodiments, an alkynylene group contains two to six carbon atoms (C₂-C₆). In some embodiments, an alkynylene group contains two to four carbon atoms (C₂-C₄). A non-limiting example of an alkynylene group is ethynylene.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Nonlimiting examples of aromatic groups include aryl and heteroaryl groups.

The terms “aryl” and “aryl group” as used interchangeably herein refer to a monovalent (i.e., having a single point of attachment to the rest of the molecule) aromatic hydrocarbon radical of 6-20 carbon atoms (C₆-C₂₀). Aryl groups can be unsubstituted or substituted. Non-limiting examples of unsubstituted and substituted aryl groups include phenyl, 2-fluorophenyl, 3-fluorophenyl, 4-fluorophenyl, 2-methylphenyl, 3-methylphenyl, 4-methylphenyl, 2-chlorophenyl, 3-chlorophenyl, 4-chlorophenyl, 2,6-dichlorophenyl, 3,4-difluorophenyl, 2-hydroxyphenyl, 3-hydroxyphenyl, 4-hydroxyphenyl, 2-methoxyphenyl, 3-methoxyphenyl, 4-methoxyphenyl, 2-phenoxyphenyl, 3-phenoxyphenyl, 4-phenoxyphenyl, 2-cyanophenyl, 3-cyanophenyl, 4-cyanophenyl, 2-dimethylaminophenyl, 3-dimethylaminophenyl, 4-dimethylaminophenyl, 3-methyl sulfonylphenyl, 4-methylsulfonylphenyl, 3-aminophenyl, 3-methylaminophenyl, 3-(2-hydroxyethoxy)phenyl, 2-trifluoromethylphenyl, 3-trifluoromethylphenyl, 4-trifluoromethylphenyl, 2-isopropylphenyl, 3-isopropylphenyl, 4-isopropylphenyl, 1-naphthyl and 2-naphthyl.

The term “heteroalkyl” as used herein refers to an alkyl group wherein at least one of the carbon atoms in the chain is replaced by a heteroatom, such as nitrogen, oxygen, phosphorous, and sulfur. A heteroalkyl group may be unsubstituted or substituted.

The terms “heterocycloalkyl,” “heterocycle,” “heterocyclyl,” and “heterocyclic group” as used interchangeably herein refer to a saturated or partially unsaturated ring system of 3 to 20 atoms, wherein at least one of the ring atoms is a heteroatom, such as nitrogen, oxygen, phosphorous, and sulfur. A heterocycloalkyl group may be unsubstituted or substituted. In some embodiments, a heterocycloalkyl group comprises 3 to 10 atoms. In some embodiments, a heterocycloalkyl group contains 3 to 7 atoms. In some embodiments, a heterocycloalkyl group is monocyclic. In some embodiments, a heterocycloalkyl group is bicyclic. In some embodiments, a heterocycloalkyl group comprises fused rings. Non-limiting examples of unsubstituted and substituted heterocycloalkyl groups include pyrrolidinyl, N-methylpyrrolidinyl, azetidinyl, dihydrofuranyl, tetrahydrofuranyl, tetrahydropyranyl, 3-hydroxypyrrolidinyl, 3-methoxypyrrolidinyl, and benzodioxolyl.

The terms “heteroaryl” and “heteroaryl group” as used interchangeably herein refer to an aromatic ring system of 3 to 20 atoms, wherein at least one of the ring atoms is a heteroatom, such as nitrogen, oxygen, phosphorous, and sulfur. A heteroaryl group may be unsubstituted or substituted. In some embodiments, a heteroaryl group contains 5 to 20 atoms. In some embodiments, a heteroaryl group contains 5 to 9 atoms. In some embodiments, a heteroaryl group contains 5 atoms. In some embodiments, a heteroaryl group contains 6 atoms. In some embodiments, a heteroaryl group contains 7 atoms. In some embodiments, a heteroaryl group is monocyclic. In some embodiments, a heteroaryl group is bicyclic. In some embodiments, a heteroaryl group contains fused rings. Non-limiting examples of heteroaryl groups include pyridinyl, imidazolyl, imidazopyridinyl, pyrimidinyl, pyrazolyl, triazolyl, pyrazinyl, tetrazolyl, furyl, 2-thienyl, 3-thienyl, isoxazolyl, thiazolyl, oxadiazolyl, 3-methyl-1,2,4-oxadiazolyl, 3-phenyl-1,2,4-oxadiazolyl, oxazolyl, isothiazolyl, pyrrolyl, quinolinyl, isoquinolinyl, tetrahydroisoquinolinyl, indolyl, benzimidazolyl, benzofuranyl, indazolyl, indolizinyl, phthalazinyl, pyridazinyl, triazinyl, thiadiazolyl, furazanyl, benzofurazanyl, benzothiophenyl, benzothiazolyl, benzoxazolyl, quinazolinyl, quinoxalinyl, naphthyridinyl, furopyridinyl, and 1H-pyrrolo[2,3-b]pyridinyl. Non-limiting examples of heteroaryl groups include:

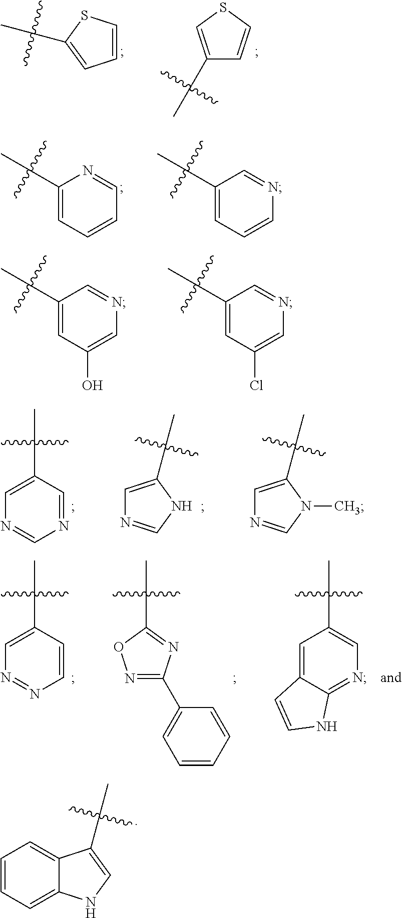

The phrase “optionally substituted” as used herein means may or may not be “substituted.” The term “substituted” as used herein refers to the replacement of one or more hydrogen atoms on a group (such as on an alkyl group, alkylene group, alkenyl group, alkenylene group, alkynyl group, alkynylene group, aryl group, heterocycloalkyl group, or heteroaryl group) by one or more substituents. Non-limiting examples of substituents that replace a single hydrogen atom include halogen, hydroxyl, and amino. Non-limiting examples of substituents that replace two hydrogen atoms include oxo and methene. Non-limiting examples of substituents that replace three hydrogen atoms include nitrile.

Additional non-limiting examples of substituents include:

C₁-C₆ linear, branched, and cyclic alkyl groups, non-limiting examples of which include methyl, ethyl, n-propyl, iso-propyl, cyclopropyl, n-butyl sec-butyl, iso-butyl, tert-butyl, cyclobutyl, cyclopentyl, and cyclohexyl;

C₂-C₈ linear, branched, and cyclic alkenyl groups, non-limiting examples of which include ethenyl (also called vinyl), 1-propenyl, and iso-propenyl;

C₂-C₈ linear and branched alkynyl groups, non-limiting examples of which include ethynyl;

substituted and unsubstituted aryl groups, non-limiting examples of which include phenyl, 2-fluorophenyl, 3-methylphenyl, 4-chlorophenyl, 2,6-dichlorophenyl, 3,4-difluorophenyl, 3-hydroxyphenyl, 4-cyanophenyl, 2-dimethylaminophenyl, 3-methyl sulfonylphenyl, 4-trifluoromethylphenyl, 3-isopropylphenyl, 1-naphthyl, and 2-naphthyl;

substituted and unsubstituted heterocyclic groups, non-limiting examples of which include pyrrolidinyl, N-methylpyrrolidinyl, azetidinyl, dihydrofuranyl, tetrahydrofuranyl, tetrahydropyranyl, 3-hydroxypyrrolidinyl, and 3-methoxypyrrolidinyl;

substituted and unsubstituted heteroaryl groups, non-limiting examples of which include pyridinyl, imidazolyl, pyrimidinyl, pyrazolyl, furyl, 2-thienyl, 3-thienyl, isoxazolyl, thiazolyl, oxadiazolyl, 3-methyl-1,2,4-oxadiazolyl, 3-phenyl-1,2,4-oxadiazolyl, indolyl, benzothiazolyl, and 1H-pyrrolo[2,3-b]pyridinyl;

—(CR^{a}R^{b})_{z}OR^{c}, non-limiting examples of which include —OH, —OCH₃, —OCH₂OH, and —OCH₂CH₃;

—(CR^{a}R^{b})_{z}N(R^{c})(R^{d}), non-limiting examples of which include —NH₂, —NHCH₃, —N(CH₃)₂, —CH₂NH₂, —CH₂NHCH₃,

a halogen atom, non-limiting examples of which include a fluorine atom (—F) and a chlorine atom (—Cl);

—(CR^{a}R^{b})_{z}CN;

—(CR^{a}R^{b})_{z}NO₂;

—CHₓX_{y}, wherein X is a halogen atom and x+y sum to 3, non-limiting examples of which include —CH₂F, —CHF₂, and —CF₃;

—(CR^{a}R^{b})_{z}C(O)R^{c}, non-limiting examples of which include —COCH₃, —COCH₂CH₃, and —CH₂COCH₃;

—(CR^{a}R^{b})_{z}C(O)OR^{c}, non-limiting examples include CO₂H, —CO₂CH₃, —CO₂CH₂CH₃, and —CH₂CO₂CH₃,

—(CR^{a}R^{b})_{z}C(O)N(R^{c})(R^{d}), non-limiting examples of which include —CONH₂, —CONHCH₃, —CON(CH₃)₂, —CH₂CONH₂, —CH₂CONHCH₃, —CH₂CON(CH₃)₂;

—(CR^{a}R^{b})_{z}SO₂R^{c}; non-limiting examples of which include —SO₂H, —SO₂CH₃, —CH₂SO₂H, —CH₂SO₂CH₃, —SO₂C₆H₅, and —CH₂SO₂C₆H₅; and

—(CR^{a}R^{b})_{z}SO₃R^{c}; non-limiting examples of which include —SO₃H, —SO₃CH₃, —CH₂SO₃H, —CH₂SO₃CH₃, —SO₃C₆H₅, and —CH₂SO₃C₆H₅;

wherein each of R^{a} and R^{b} is independently chosen from hydrogen and substituted or unsubstituted C₁-C₆ linear, branched, or cyclic alkyl, each of R^{c} and R^{d} is independently chosen from hydrogen, substituted or unsubstituted C₁-C₆ linear, branched, or cyclic alkyl, and aryl, or wherein R^{c} and R^{d} together form a ring system comprising 3 to 7 atoms, and z is chosen from 0, 1, 2, 3, and 4.

As used herein, the term “pharmaceutical composition” refers to a preparation that is in such form as to permit the biological activity of the active ingredient to be effective, and that contains no additional components that are unacceptably toxic to a subject to which the composition would be administered. In some embodiments, such compositions may be sterile.

The term “pharmaceutically acceptable,” as used herein in “pharmaceutically acceptable salt” and “pharmaceutically acceptable excipient,” refers to a component that is, within the scope of sound medical judgment, suitable for use in contact with the tissues of humans and other mammals without undue toxicity, irritation, allergic response and the like, and are commensurate with a reasonable benefit/risk ratio.

The phrase “pharmaceutically acceptable excipient” is employed herein to refer to a pharmaceutically acceptable material chosen from a solvent, dispersion media, diluent, dispersion, suspension aid, surface active agent, isotonic agent, thickening or emulsifying agent, preservative, polymer, peptide, protein, cell, hyaluronidase, and mixtures thereof. In some embodiments, the solvent is an aqueous solvent.

“Treatment,” “treat,” and “treating” refer to reversing, alleviating (e.g., alleviating one or more symptoms), and/or delaying the progression of a medical condition or disorder described herein.

The terms “disease” and “disorder” are used interchangeably herein and refer to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, sickness, illness, complaint, indisposition, or affection.

“Subject,” as used herein, means an animal subject, such as a mammalian subject, and particularly human beings.

As used herein, the term “administering” refers to the placement of a compound, pharmaceutically acceptable salt thereof, and/or a pharmaceutical composition comprising into a mammalian tissue or a subject by a method or route that results in at least partial localization of the compound, salt, and/or composition at a desired site or tissue location.

The term “therapeutically effective amount” as used herein refers to an amount of a compound or salt that produces a desired effect for which it is administered (e.g., improvement in symptoms of a disease or condition mediated by AhR signaling, lessening the severity of such a disease or condition or a symptom thereof, and/or reducing progression any one of the foregoing). The exact amount of an effective dose will depend on the purpose of the treatment and will be ascertainable by one skilled in the art using known techniques (see, e.g., Lloyd (1999) The Art, Science and Technology of Pharmaceutical Compounding).

One of ordinary skill in the art would recognize that, when an amount of a compound is disclosed, the relevant amount of a pharmaceutically acceptable salt form of the compound is an amount equivalent to the amount of the free base of the compound. The amounts of the compounds and pharmaceutically acceptable salts disclosed herein are based upon the free base form of the relevant compound. For example, “10 mg of at least one entity chosen from compounds of Formulas I or Ia and pharmaceutically acceptable salts thereof” refers to 10 mg of a compound of Formulas I or Ia or an amount of a pharmaceutically acceptable salt of the compound of Formulas I or Ia equivalent to 10 mg of the relevant compound of Formulas I or Ia.

The “effectiveness” of a compound or composition of the disclosure can be assessed by any method known to one of ordinary skill in the art, including those described in the examples of this disclosure. Effectiveness can be established in vitro (biochemical and/or biological in cultured cells) and/or in vivo. Effectiveness in vitro may be used to extrapolate or predict some degree of effectiveness in vivo, in an animal or in a human subject. A reference or standard or comparison may be used. The term “effective” at inhibiting a receptor (such as AhR), and/or signaling mediated by the enzyme in the context of this disclosure and claims means reducing/activating the activity of the receptor and/or the activation and propagation of the signaling pathway in terms of activation of a downstream molecule or known biological effect by a detectable or measurable amount relative to the baseline activity. This can be assessed in vitro or in vivo and, in some cases, extrapolated to what an activity or benefit in vivo might be by one of ordinary skill in the art. In some embodiments, the reduction or activation is measured in terms of percentage reduction or activation, relative to the activity in the absence of exposure to the compound of the disclosure, including, for example, at least 5%, at least 10%, 20%, at least 30%, at least 40%, at least 50%, at least 60%, at least 70%, at least 80%, at least 90%, or about 100%. The activity might also fall within a range, e.g., 5-10%, 10-20%, and any other range interval between 1% and 100%. An amount is “effective” in vivo if it produces any benefit to the subject to whom the compound or salt is administered.

Disclosed herein are compounds chosen from

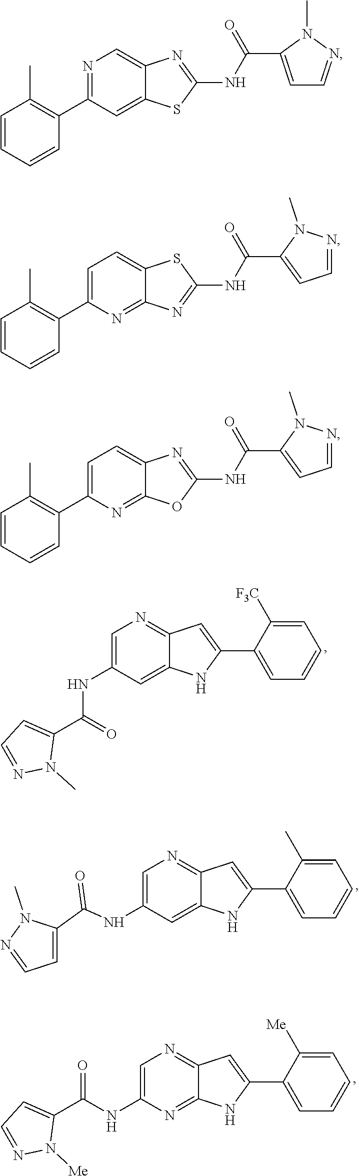 [SMILES: Cc1ccccc1-c1cc2sc(NC(=O)c3ccnn3C)nc2cn1 ; Cc1ccccc1-c1ccc2sc(NC(=O)c3ccnn3C)nc2n1 ; Cc1ccccc1-c1ccc2nc(NC(=O)c3ccnn3C)oc2n1 ; Cn1nccc1C(=O)Nc1cnc2cc(-c3ccccc3C(F)(F)F)[nH]c2c1 ; Cc1ccccc1-c1cc2ncc(NC(=O)c3ccnn3C)cc2[nH]1 ; Cc1ccccc1-c1cc2ncc(NC(=O)c3ccnn3C)nc2[nH]1]

and pharmaceutically acceptable salts of any of the foregoing.

Also disclosed herein are compounds chosen from

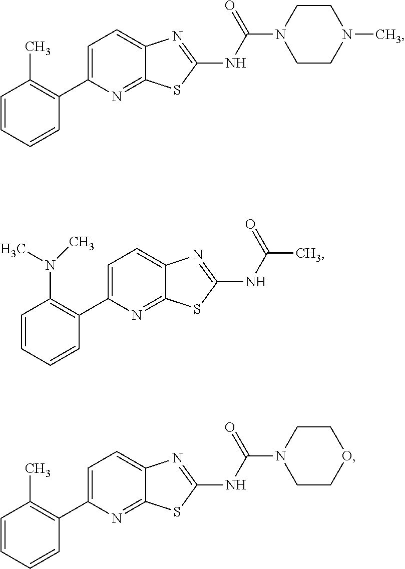 [SMILES: Cc1ccccc1-c1ccc2nc(NC(=O)N3CCN(C)CC3)sc2n1 ; CC(=O)Nc1nc2ccc(-c3ccccc3N(C)C)nc2s1 ; Cc1ccccc1-c1ccc2nc(NC(=O)N3CCOCC3)sc2n1]

and pharmaceutically acceptable salts of any of the foregoing.

In some embodiments, there is provided a pharmaceutical composition comprising at least one entity chosen from compounds of any of the foregoing and pharmaceutically acceptable salts thereof, and at least one pharmaceutically acceptable excipient.

In some embodiments, there is provided a method of treating a disease or condition associated with aberrant AhR signaling in a subject in need thereof comprising administering to the subject a pharmaceutical composition comprising at least one entity chosen from compounds of any of the foregoing or a therapeutically effective amount of at least one entity chosen from compounds of formula I

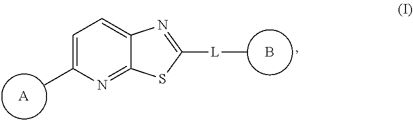

compounds of formula (II)

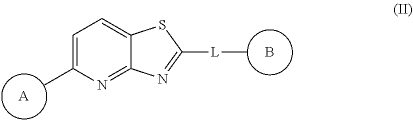

with the proviso that the compound is not

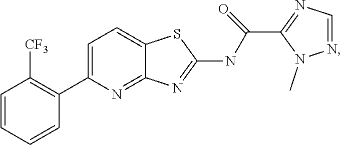 [SMILES: Cn1ncnc1C(=O)Nc1nc2nc(-c3ccccc3C(F)(F)F)ccc2s1]

compounds of formula (III)

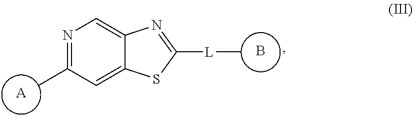

compounds of formula (IV)

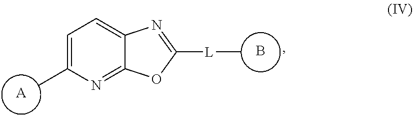

compounds of formula (V)

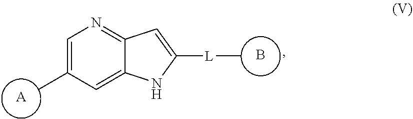

compounds of formula (VI)

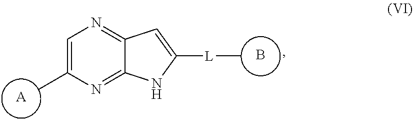

compounds of formula (VII)

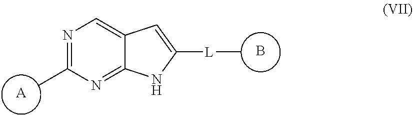

and pharmaceutically acceptable salts of any of the foregoing,
wherein

Ring A is chosen from optionally substituted aryls, optionally substituted heteroaryls, optionally substituted cycloalkyls, and optionally substituted heterocycloalkyls;

Ring B is chosen from optionally substituted aryls, optionally substituted heteroaryls, optionally substituted cycloalkyls, and optionally substituted heterocycloalkyls; and

L is chosen from a bond and —NT¹-C(O)—, wherein T¹ is H or Me.

In some embodiments, Ring A is chosen from optionally substituted 6-10 membered aryls. In some embodiments, Ring A is chosen from optionally substituted 5-10 membered heteroaryls. In some embodiments, Ring A is chosen from optionally substituted 3-10 membered cycloalkyls. In some embodiments, Ring A is chosen from optionally substituted 3-10 membered heterocycloalkyls.

In some embodiments, Ring A is an optionally substituted phenyl. In some embodiments, Ring A is an optionally substituted pyrrolyl. In some embodiments, Ring A is an optionally substituted furanyl. In some embodiments, Ring A is an optionally substituted furazanyl. In some embodiments, Ring A is an optionally substituted thiophenyl. In some embodiments, Ring A is an optionally substituted imidazolyl. In some embodiments, Ring A is an optionally substituted isothiazoyl. In some embodiments, Ring A is an optionally substituted isoxazolyl. In some embodiments, Ring A is an optionally substituted oxazolyl. In some embodiments, Ring A is an optionally substituted oxadiazolyl. In some embodiments, Ring A is an optionally substituted tetrazolyl. In some embodiments, Ring A is an optionally substituted thiazolyl. In some embodiments, Ring A is an optionally substituted triazolyl. In some embodiments, Ring A is an optionally substituted pyrazolyl. In some embodiments, Ring A is an optionally substituted pyridinyl. In some embodiments, Ring A is an optionally substituted pyrazinyl. In some embodiments, Ring A is an optionally substituted pyridazinyl. In some embodiments, Ring A is an optionally substituted pyrimidinyl.

In some embodiments, Ring A is

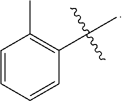 [SMILES: *c1ccccc1C]

In some embodiments, Ring A is

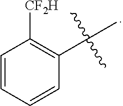 [SMILES: *c1ccccc1C(F)F]

In some embodiments, Ring A is

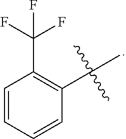 [SMILES: *c1ccccc1C(F)(F)F]

In some embodiments, Ring A is

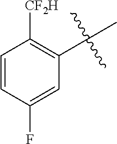 [SMILES: *c1cc(F)ccc1C(F)F]

In some embodiments, Ring A is

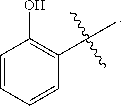 [SMILES: *c1ccccc1O]

In some embodiments, Ring A is

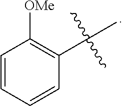 [SMILES: *c1ccccc1OC]

In some embodiments, Ring A is

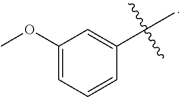 [SMILES: *c1cccc(OC)c1]

In some embodiments, Ring A is

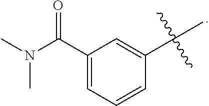 [SMILES: *c1cccc(C(=O)N(C)C)c1]

In some embodiments, Ring A is

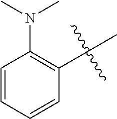 [SMILES: *c1ccccc1N(C)C]

In some embodiments, Ring A is

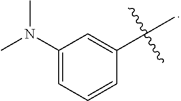 [SMILES: *c1cccc(N(C)C)c1]

In some embodiments, Ring A is

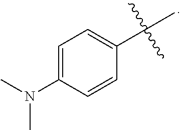 [SMILES: *c1ccc(N(C)C)cc1]

In some embodiments, Ring A is

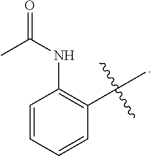 [SMILES: *c1ccccc1NC(C)=O]

In some embodiments, Ring A is

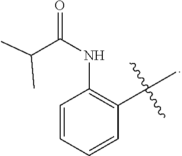 [SMILES: *c1ccccc1NC(=O)C(C)C]

In some embodiments, Ring A is

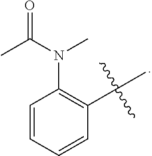 [SMILES: *c1ccccc1N(C)C(C)=O]

In some embodiments, Ring A is

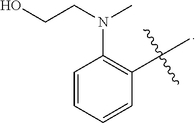 [SMILES: *c1ccccc1N(C)CCO]

In some embodiments, Ring A is

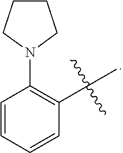 [SMILES: *c1ccccc1N1CCCC1]

In some embodiments, Ring A is

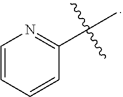 [SMILES: *c1ccccn1]

In some embodiments, Ring A is

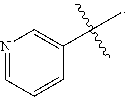 [SMILES: *c1cccnc1]

In some embodiments, Ring A is

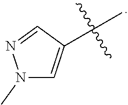 [SMILES: *c1cnn(C)c1]

In some embodiments, Ring A is

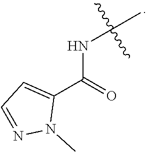 [SMILES: *NC(=O)c1ccnn1C]

In some embodiments, Ring A is

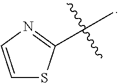 [SMILES: *c1nccs1]

In some embodiments, Ring A is

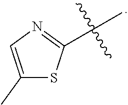 [SMILES: *c1ncc(C)s1]

In some embodiments, Ring A is

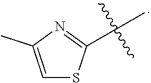 [SMILES: *c1nc(C)cs1]

In some embodiments, Ring A is

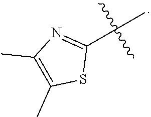 [SMILES: *c1nc(C)c(C)s1]

In some embodiments, Ring A is

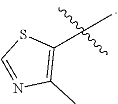 [SMILES: *c1scnc1C]

In some embodiments, Ring A is

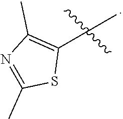 [SMILES: *c1sc(C)nc1C]

In some embodiments, Ring A is

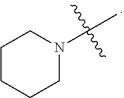 [SMILES: *N1CCCCC1]

In some embodiments, Ring B is chosen from optionally substituted 6-10 membered aryls. In some embodiments, Ring B is chosen from optionally substituted 5-10 membered heteroaryls. In some embodiments, Ring B is chosen from optionally substituted 3-10 membered cycloalkyls. In some embodiments, Ring B is chosen from optionally substituted 3-10 membered heterocycloalkyls.

In some embodiments, Ring B is an optionally substituted phenyl. In some embodiments, Ring B is an optionally substituted pyrrolyl. In some embodiments, Ring B is an optionally substituted furanyl. In some embodiments, Ring B is an optionally substituted furazanyl. In some embodiments, Ring B is an optionally substituted thiophenyl. In some embodiments, Ring B is an optionally substituted imidazolyl. In some embodiments, Ring B is an optionally substituted isothiazoyl. In some embodiments, Ring B is an optionally substituted isoxazolyl. In some embodiments, Ring B is an optionally substituted oxazolyl. In some embodiments, Ring B is an optionally substituted oxadiazolyl. In some embodiments, Ring B is an optionally substituted tetrazolyl. In some embodiments, Ring B is an optionally substituted thiazolyl. In some embodiments, Ring B is an optionally substituted triazolyl. In some embodiments, Ring B is an optionally substituted pyrazolyl. In some embodiments, Ring B is an optionally substituted pyridinyl. In some embodiments, Ring B is an optionally substituted pyrazinyl. In some embodiments, Ring B is an optionally substituted pyridazinyl. In some embodiments, Ring B is an optionally substituted pyridinonyl. In some embodiments, Ring B is an optionally substituted pyrimidinyl. In some embodiments, Ring B is an optionally substituted piperidinyl. In some embodiments, Ring B is an optionally substituted piperazinyl. In some embodiments, Ring B is an optionally substituted morpholinyl.

In some embodiments, Ring B is

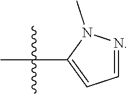 [SMILES: *c1ccnn1C]

In some embodiments, Ring B is

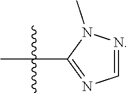 [SMILES: *c1ncnn1C]

In some embodiments, Ring B is

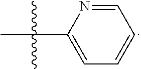 [SMILES: *c1ccccn1]

In some embodiments, Ring B is

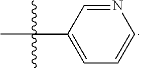 [SMILES: *c1cccnc1]

In some embodiments, Ring B is

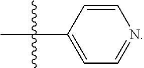 [SMILES: *c1ccncc1]

In some embodiments, Ring B is

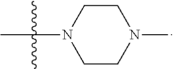 [SMILES: *N1CCN(C)CC1]

In some embodiments, Ring B is

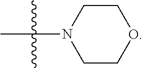 [SMILES: *N1CCOCC1]

In some embodiments, Ring B is

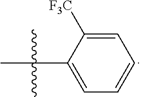 [SMILES: *c1ccccc1C(F)(F)F]

In some embodiments, L is a bond. In some embodiments, L is —NH(C═O)—. In some embodiments L is —NCH₃(C═O)—.

In some embodiments, in the methods disclosed herein, the least one entity is chosen from:
1-Methyl-N-(5-(2-(trifluoromethyl)phenyl)thiazolo[5,4-b]pyridin-2-yl)-1H-pyrazole-5-carboxamide;
1-Methyl-N-(5-(2-(trifluoromethyl)phenyl)thiazolo[5,4-b]pyridin-2-yl)-1H-1,2,4-triazole-5-carboxamide;
1-Methyl-N-(5-(o-tolyl)thiazolo[5,4-b]pyridin-2-yl)-1H-1,2,4-triazole-5-carboxamide;
N-(5-(2-(difluoromethyl)phenyl)thiazolo[5,4-b]pyridin-2-yl)-1-methyl-1H-pyrazole-5-carboxamide;
N-(5-(2-(difluoromethyl)phenyl)thiazolo[5,4-b]pyridin-2-yl)-1-methyl-1H-1,2,4-triazole-5-carboxamide;
N-(5-(o-tolyl)thiazolo[5,4-b]pyridin-2-yl)picolinamide;
N-(5-(o-tolyl)thiazolo[5,4-b]pyridin-2-yl)nicotinamide;
N-(5-(o-tolyl)thiazolo[5,4-b]pyridin-2-yl)isonicotinamide;
N-(5-(2-methoxyphenyl)thiazolo[5,4-b]pyridin-2-yl)-1-methyl-1H-1,2,4-triazole-5-carboxamide;
N-(5-(2-(difluoromethyl)-5-fluorophenyl)thiazolo[5,4-b]pyridin-2-yl)-1-methyl-1H-pyrazole-5-carboxamide;
N-(5-(2-(difluoromethyl)-5-fluorophenyl)thiazolo[5,4-b]pyridin-2-yl)-1-methyl-1H-1,2,4-triazole-5-carboxamide;
N-(5-(2-methoxyphenyl)thiazolo[5,4-b]pyridin-2-yl)picolinamide;
N-(5-(2-methoxyphenyl)thiazolo[5,4-b]pyridin-2-yl)nicotinamide;
N-(5-(2-methoxyphenyl)thiazolo[5,4-b]pyridin-2-yl)isonicotinamide;
1-Methyl-N-(5-(o-tolyl)thiazolo[5,4-b]pyridin-2-yl)-1H-pyrazole-5-carboxamide;
N-(5-(3-methoxyphenyl)thiazolo[5,4-b]pyridin-2-yl)-1-methyl-1H-pyrazole-5-carboxamide;
N-(5-(3-(dimethylcarbamoyl)phenyl)thiazolo[5,4-b]pyridin-2-yl)-1-methyl-1H-pyrazole-5-carboxamide;
N-(5-(2-(dimethylamino)phenyl)thiazolo[5,4-b]pyridin-2-yl)-1-methyl-1H-pyrazole-5-carboxamide;
N-(5-(3-(dimethylamino)phenyl)thiazolo[5,4-b]pyridin-2-yl)-1-methyl-1H-pyrazole-5-carboxamide;
N-(5-(4-(dimethylamino)phenyl)thiazolo[5,4-b]pyridin-2-yl)-1-methyl-1H-pyrazole-5-carboxamide;
1-Methyl-N-(5-(2-(pyrrolidin-1-yl)phenyl)thiazolo[5,4-b]pyridin-2-yl)-1H-pyrazole-5-carboxamide;
1-Methyl-N-(5-(1-methyl-1H-pyrazol-4-yl)thiazolo[5,4-b]pyridin-2-yl)-1H-pyrazole-5-carboxamide;
N-(5-(2-(dimethylamino)phenyl)thiazolo[5,4-b]pyridin-2-yl)acetamide;
N-(5-(2-hydroxyphenyl)thiazolo[5,4-b]pyridin-2-yl)-1-methyl-1H-pyrazole-5-carboxamide;
N-(5-(2-methoxyphenyl)thiazolo[5,4-b]pyridin-2-yl)-1-methyl-1H-pyrazole-5-carboxamide;
1-Methyl-N-(5-(pyridin-3-yl)thiazolo[5,4-b]pyridin-2-yl)-1H-pyrazole-5-carboxamide;
1-Methyl-N-(5-(pyridin-2-yl)thiazolo[5,4-b]pyridin-2-yl)-1H-pyrazole-5-carboxamide;
1-Methyl-N-(5-(thiazol-2-yl)thiazolo[5,4-b]pyridin-2-yl)-1H-pyrazole-5-carboxamide;
N-(5-(4,5-dimethylthiazol-2-yl)thiazolo[5,4-b]pyridin-2-yl)-1-methyl-1H-pyrazole-5-carboxamide;
1-Methyl-N-(5-(5-methylthiazol-2-yl)thiazolo[5,4-b]pyridin-2-yl)-1H-pyrazole-5-carboxamide;
1-Methyl-N-(5-(4-methylthiazol-5-yl)thiazolo[5,4-b]pyridin-2-yl)-1H-pyrazole-5-carboxamide;
1-Methyl-N-(5-(4-methylthiazol-2-yl)thiazolo[5,4-b]pyridin-2-yl)-1H-pyrazole-5-carboxamide;
N-(5-(2,4-dimethylthiazol-5-yl)thiazolo[5,4-b]pyridin-2-yl)-1-methyl-1H-pyrazole-5-carboxamide;
N-(5-(2-isobutyramidophenyl)thiazolo[5,4-b]pyridin-2-yl)-1-methyl-1H-pyrazole-5-carboxamide;
N-(5-(2-acetamidophenyl)thiazolo[5,4-b]pyridin-2-yl)-1-methyl-1H-pyrazole-5-carboxamide;
1-Methyl-N-(5-(2-(N-methylacetamido)phenyl)thiazolo[5,4-b]pyridin-2-yl)-1H-pyrazole-5-carboxamide;
N-(5-(2-(2-hydroxyethyl)(methyl)amino)phenyl)thiazolo[5,4-b]pyridin-2-yl)-1-methyl-1H-pyrazole-5-carboxamide;
N-Methyl-N-(5-(o-tolyl)thiazolo[5,4-b]pyridin-2-yl)picolinamide;
N-(5-(o-tolyl)thiazolo[5,4-b]pyridin-2-yl)morpholine-4-carboxamide;
4-Methyl-N-(5-(o-tolyl)thiazolo[5,4-b]pyridin-2-yl)piperazine-1-carboxamide; and
pharmaceutically acceptable salts of any of the foregoing.

In some embodiments, there is provided a method of treating a disease or condition associated with aberrant AhR signaling in a subject in need thereof comprising administering to the subject a therapeutically effective amount of a pharmaceutical composition comprising at least one entity chosen from compounds of any of the foregoing or a therapeutically effective amount of at least one entity chosen from any of the foregoing compounds.

In some embodiments, there is provided a method of treating a disease or condition mediated by AhR signaling in a subject in need thereof comprising administering to the subject a therapeutically effective amount of a pharmaceutical composition comprising at least one entity chosen from compounds of any of the foregoing or a therapeutically effective amount of at least one entity chosen from any of the foregoing compounds.

In some embodiments, there is provided a method of inhibiting cancer cell proliferation mediated by AhR signaling in a subject in need thereof comprising administering to the subject a pharmaceutical composition comprising at least one entity chosen from compounds of any of the foregoing or a therapeutically effective amount of at least one entity chosen from any of the foregoing compounds.

In some embodiments, there is provided a method of inhibiting tumor cell invasion or metastasis mediated by AhR signaling in a subject in need thereof comprising administering to the subject a therapeutically effective amount of a pharmaceutical composition comprising at least one entity chosen from compounds of any of the foregoing or a therapeutically effective amount of at least one entity chosen from any of the foregoing compounds.

In some embodiments, the disease is chosen from cancers. In some embodiments, the disease is chosen from breast cancers, respiratory tract cancers, brain cancers, cancers of reproductive organs, digestive tract cancers, urinary tract cancers, eye cancers, liver cancers, skin cancers, head and neck cancers, thyroid cancers, parathyroid cancers, and metastases of any of the foregoing. In some embodiments, the disease is chosen from lymphomas, sarcomas, melanomas, glioblastomas, and leukemias.

In some embodiments, the method comprises further administering to the subject a therapeutically effective amount of at least one immune checkpoint inhibitor. In some embodiments, the immune checkpoint inhibitor is chosen from PD-1 inhibitors. In some embodiments the immune checkpoint inhibitors is chosen from PD-L1 inhibitors. In some embodiments the immune checkpoint inhibitors is chosen from CTLA-4 blockers.

The compounds of the present disclosure and pharmaceutically acceptable salts thereof can be incorporated into pharmaceutical compositions. In some embodiments, the disclosure is drawn to a pharmaceutical composition comprising at least one entity chosen from compounds of the present disclosure and pharmaceutically acceptable salts thereof. In some embodiments, the disclosure is drawn to a pharmaceutical composition consisting essentially of at least one entity chosen from compounds of the pres and pharmaceutically acceptable salts thereof.

In some embodiments, the pharmaceutical composition comprises at least one entity chosen from compounds of the present disclosure and pharmaceutically acceptable salts thereof and at least one pharmaceutically acceptable excipient. Pharmaceutically acceptable excipients are well-known to persons having ordinary skill in the art and are described in, as a non-limiting example, Remington: The Science and Practice of Pharmacy, 22nd Edition, Lippincott Williams & Wilkins, Philadelphia, Pa. (2013) and any other editions, which are hereby incorporated by reference.

Compounds of the disclosure, pharmaceutically acceptable salts thereof, and/or pharmaceutical compositions comprising said at least one entity chosen from compounds disclosed herein and pharmaceutically acceptable salts thereof can be used in therapeutic treatments.

The compounds, pharmaceutically acceptable salts, and/or pharmaceutical compositions can be administered in unit forms of administration to mammalian subjects, including human beings. Suitable non-limiting examples of unit forms of administration include orally administered forms and forms administered via a parenteral/systemic route, non-limiting examples of which including inhalation, subcutaneous administration, intramuscular administration, intravenous administration, intradermal administration, and intravitreal administration.

In some embodiments, pharmaceutical compositions suitable for oral administration can be in the form of tablets, pills, powders, hard gelatine capsules, soft gelatine capsules, and/or granules. In some embodiments of such pharmaceutical compositions, a compound of the disclosure and/or a pharmaceutically acceptable salt of a compound of the disclosure is (or are) mixed with one or more inert diluents, non-limiting examples of which including starch, cellulose, sucrose, lactose, and silica. In some embodiments, such pharmaceutical compositions may further comprise one or more substances other than diluents, such as (as non-limiting examples), lubricants, coloring agents, coatings, or varnishes.

In some embodiments, pharmaceutical compositions for parenteral administration can be in the form of aqueous solutions, non-aqueous solutions, suspensions, emulsions, drops, or any combination(s) thereof. In some embodiments, such pharmaceutical compositions may comprise one or more of water, pharmaceutically acceptable glycol(s), pharmaceutically acceptable oil(s), pharmaceutically acceptable organic esters, or other pharmaceutically acceptable solvents.

In some embodiments, disclosed herein is a method of inhibiting AhR comprising administering to a subject in need thereof at least one entity chosen from compounds of formula I or formula Ia and pharmaceutically acceptable salts thereof. In some embodiments, disclosed herein is a method of reducing the activity of AhR comprising administering to a subject in need thereof at least one entity chosen from compounds of formula I or formula Ia and pharmaceutically acceptable salts thereof.

In some embodiments, disclosed herein is a method of treating a cancer comprising administering to a subject in need thereof at least one entity chosen from compounds of formula I or formula Ia and pharmaceutically acceptable salts thereof. In some embodiments, the cancers are chosen from liquid tumors and solid tumors. In some embodiments, the cancer is chosen from breast cancers, respiratory tract cancers, brain cancers, cancers of reproductive organs, digestive tract cancers, urinary tract cancers, eye cancers, liver cancers, skin cancers, head and neck cancers, thyroid cancers, parathyroid cancers, and metastases of any of the foregoing. In some embodiments, the cancers are chosen from breast cancers, pancreatic cancers, prostate cancers, and colon cancers. In some embodiments, the cancers are chosen from lymphomas, sarcomas, and leukemias.

In some embodiments, disclosed herein is a method of treating ocular disorders comprising administering to a subject in need thereof at least one entity chosen from compounds of formula I or formula Ia and pharmaceutically acceptable salts thereof.

With regard to the methods disclosed herein, the mode (or modes) of administration, dose (or doses), and pharmaceutical form (or forms) can be determined according to criteria generally considered during the establishment of a treatment of a patient, such as, by way of non-limiting examples, the potency of the compound(s) and/or pharmaceutically acceptable salts of the compound(s), the age of the patient, the body weight of the patient, the severity of the patient's condition (or conditions), the patient's tolerance to the treatment, and secondary effects observed in treatment. Determination of doses effective to provide therapeutic benefit for specific modes and frequency of administration is within the capabilities of those skilled in the art.

In some embodiments, a compound of the disclosure and/or pharmaceutically acceptable salt thereof is present in a pharmaceutical composition in an amount ranging from 5 μg to 2,000 mg. In some embodiments, a compound of the disclosure and/or pharmaceutically acceptable salt thereof is present in a pharmaceutical composition in an amount ranging from 5 μg to 1,000 mg. In some embodiments, a compound of the disclosure and/or pharmaceutically acceptable salt thereof is present in a pharmaceutical composition in an amount ranging from 5 μg to 500 mg. In some embodiments, a compound of the disclosure and/or pharmaceutically acceptable salt thereof is present in a pharmaceutical composition in an amount ranging from 5 μg to 250 mg. In some embodiments, a compound of the disclosure and/or pharmaceutically acceptable salt thereof is present in a pharmaceutical composition in an amount ranging from 5 μg to 100 mg. In some embodiments, a compound of the disclosure and/or pharmaceutically acceptable salt thereof is present in a pharmaceutical composition in an amount ranging from 5 μg to 50 mg.

In some embodiments, a compound of the disclosure and/or pharmaceutically acceptable salt thereof is present in a pharmaceutical composition in an amount ranging from 1 mg to 5,000 mg. In some embodiments, a compound of the disclosure and/or pharmaceutically acceptable salt thereof is present in a pharmaceutical composition in an amount ranging from 1 mg to 3,000 mg. In some embodiments, a compound of the disclosure and/or pharmaceutically acceptable salt thereof is present in a pharmaceutical composition in an amount ranging from 1 mg to 2,000 mg. In some embodiments, a compound of the disclosure and/or pharmaceutically acceptable salt thereof is present in a pharmaceutical composition in an amount ranging from 1 mg to 1,000 mg. In some embodiments, a compound of the disclosure and/or pharmaceutically acceptable salt thereof is present in a pharmaceutical composition in an amount ranging from 1 mg to 500 mg. In some embodiments, a compound of the disclosure and/or pharmaceutically acceptable salt thereof is present in a pharmaceutical composition in an amount ranging from 1 mg to 250 mg. In some embodiments, a compound of the disclosure and/or pharmaceutically acceptable salt thereof is present in a pharmaceutical composition in an amount ranging from 1 mg to 100 mg. In some embodiments, a compound of the disclosure and/or pharmaceutically acceptable salt thereof is present in a pharmaceutical composition in an amount ranging from 1 mg to 50 mg.

In some embodiments, a compound of the disclosure and/or pharmaceutically acceptable salt thereof is present in a pharmaceutical composition in an amount of 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, or 5,000 mg.

Effective amounts and dosages can be estimated initially from in vitro assays. For example, an initial dosage for use in animals can be formulated to achieve a circulating blood or serum concentration of active compound that is at or above an IC₅₀ of the particular compound as measured in an in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” in Goodman and Gilman's The Pharmaceutical Basis of Therapeutics, Chapter 1, pp. 1-46, latest edition, Pergamagon Press, and the references cited therein, which methods are incorporated herein by reference in their entirety. Initial dosages can also be estimated from in vivo data, such as animal models. Animal models useful for testing the efficacy of compounds to treat or prevent the various diseases described in this disclosure are well-known in the art.

In some embodiments, the administered dose ranges from 0.0001 or 0.001 or 0.01 mg/kg/day to 100 mg/kg/day, but can be higher or lower, depending upon, among other factors, the activity of the compound, its bioavailability, the mode of administration and various factors discussed above. Doses and intervals can be adjusted individually to provide plasma levels of the compound(s) which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Non-limiting embodiments of the present disclosure include:

1. A compound chosen from

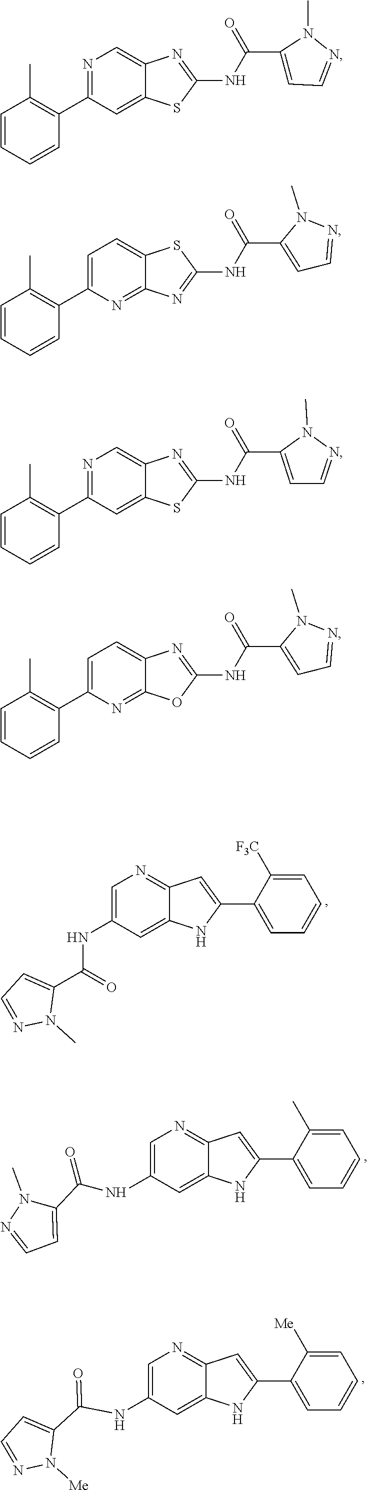

and pharmaceutically acceptable salts of any of the foregoing.
2. A compound chosen from

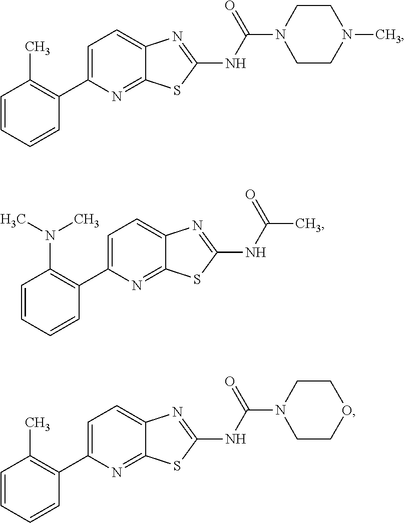 [SMILES: Cc1ccccc1-c1ccc2nc(NC(=O)N3CCN(C)CC3)sc2n1 ; CC(=O)Nc1nc2ccc(-c3ccccc3N(C)C)nc2s1 ; Cc1ccccc1-c1ccc2nc(NC(=O)N3CCOCC3)sc2n1]

and pharmaceutically acceptable salts of any of the foregoing.
3. A pharmaceutical composition comprising at least one entity chosen from compounds of any one of embodiments 1 and 2 and pharmaceutically acceptable salts thereof, and at least one pharmaceutically acceptable excipient.
4. A method of treating a disease or condition associated with aberrant AhR signaling in a subject in need thereof comprising administering to the subject a pharmaceutical composition according to embodiment 3 or a therapeutically effective amount of at least one entity chosen from
compounds of embodiment 1,
compounds of embodiment 2,
compounds of formula I

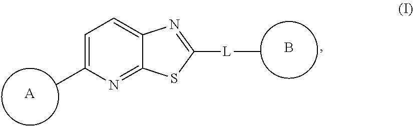

compounds of formula (II)

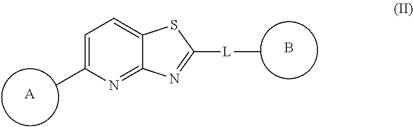

with the proviso that the compound is not

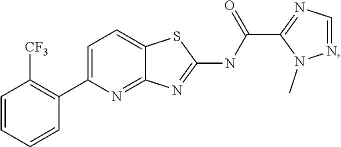 [SMILES: Cn1ncnc1C(=O)Nc1nc2nc(-c3ccccc3C(F)(F)F)ccc2s1]

compounds of formula (III)

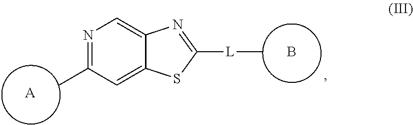

compounds of formula (IV)

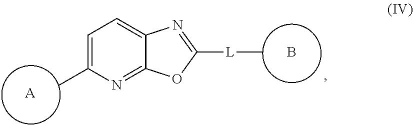

compounds of formula (V)

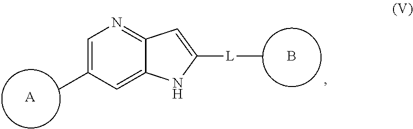

compounds of formula (VI)

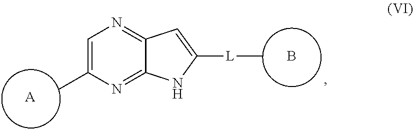

compounds of formula (VII)

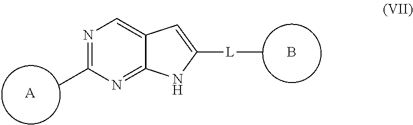

and pharmaceutically acceptable salts of any of the foregoing,
wherein

Ring A is chosen from optionally substituted aryls, optionally substituted heteroaryls, optionally substituted cycloalkyls, and optionally substituted heterocycloalkyls;

Ring B is chosen from optionally substituted aryls, optionally substituted heteroaryls, optionally substituted cycloalkyls, and optionally substituted heterocycloalkyls; and

L is chosen from a bond and —NT¹-C(O)—, wherein T¹ is H or Me.

5. A method of treating a disease or condition associated with aberrant AhR signaling in a subject in need thereof comprising administering to the subject a therapeutically effective amount of a pharmaceutical composition according to embodiment 3 or a therapeutically effective amount of at least one entity chosen from compounds of any one of embodiments 1 and 2, compounds of Formula I

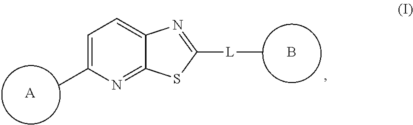

compounds of formula (II)

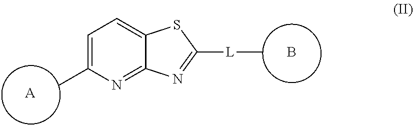

with the proviso that the compound is not

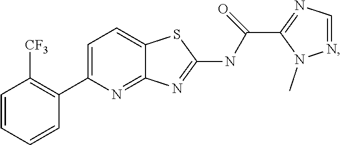 [SMILES: Cn1ncnc1C(=O)Nc1nc2nc(-c3ccccc3C(F)(F)F)ccc2s1]

compounds of formula (III)

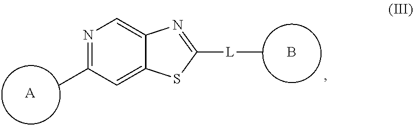

compounds of formula (IV)

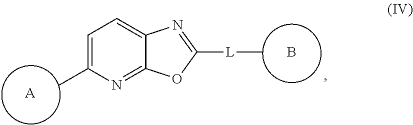

compounds of formula (V)

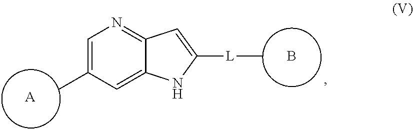

compounds of formula (VI)

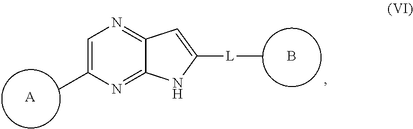

compounds of formula (VII)

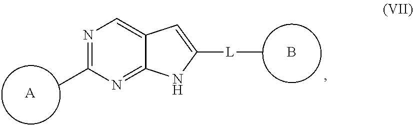

and pharmaceutically acceptable salts thereof,
wherein

Ring A is chosen from optionally substituted aryls, optionally substituted heteroaryls, optionally substituted cycloalkyls, and optionally substituted heterocycloalkyls;

Ring B is chosen from optionally substituted aryls, optionally substituted heteroaryls, optionally substituted cycloalkyls, and optionally substituted heterocycloalkyls; and

L is chosen from a bond and —NT¹-C(O)—, wherein T¹ is H or Me.

6. A method of treating a disease or condition mediated by AhR signaling in a subject in need thereof comprising administering to the subject a therapeutically effective amount of a pharmaceutical composition according to embodiment 3 or a therapeutically effective amount of at least one entity chosen from compounds of any one of embodiments 1 and 2, compounds of Formula I

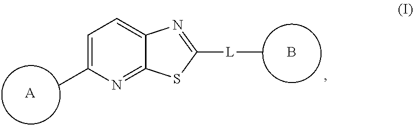

compounds of formula (II)

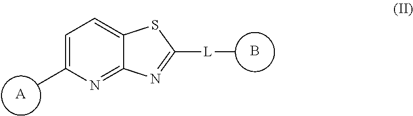

with the proviso that the compound is not

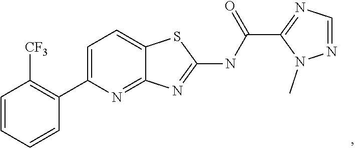 [SMILES: Cn1ncnc1C(=O)Nc1nc2nc(-c3ccccc3C(F)(F)F)ccc2s1]

compounds of formula (III)

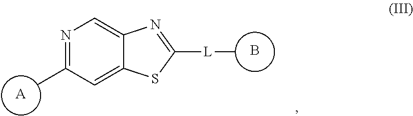

compounds of formula (IV)

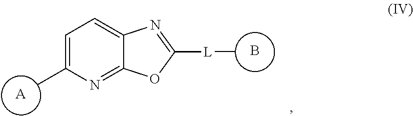

compounds of formula (V)

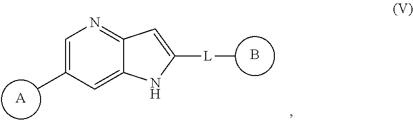

compounds of formula (VI)

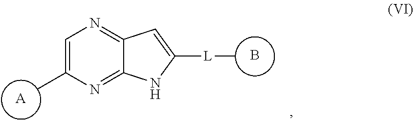

compounds of formula (VII)

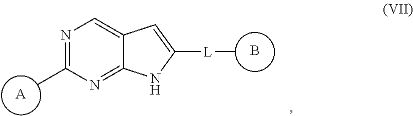

and pharmaceutically acceptable salts thereof,
wherein

Ring A is chosen from optionally substituted aryls, optionally substituted heteroaryls, optionally substituted cycloalkyls, and optionally substituted heterocycloalkyls;

Ring B is chosen from optionally substituted aryls, optionally substituted heteroaryls, optionally substituted cycloalkyls, and optionally substituted heterocycloalkyls; and

L is chosen from a bond and —NT¹-C(O)—, wherein T¹ is H or Me.

7. A method of inhibiting cancer cell proliferation mediated by AhR signaling in a subject in need thereof comprising administering to the subject a pharmaceutical composition according to embodiment 3 or a therapeutically effective amount of at least one entity chosen from compounds of any one of embodiments 1 and 2, compounds of Formula I

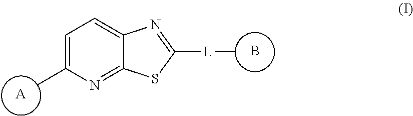

compounds of formula (II)

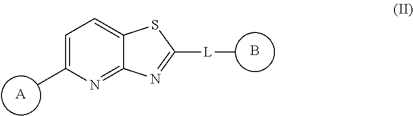

with the proviso that the compound is not

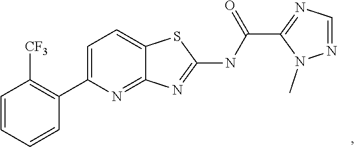 [SMILES: Cn1ncnc1C(=O)Nc1nc2nc(-c3ccccc3C(F)(F)F)ccc2s1]

compounds of formula (III)

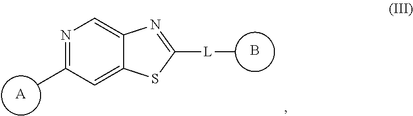

compounds of formula (IV)

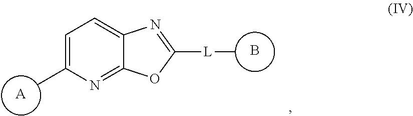

compounds of formula (V)

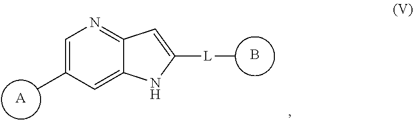

compounds of formula (VI)

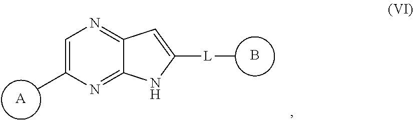

compounds of formula (VII)

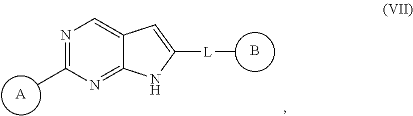

and pharmaceutically acceptable salts thereof,
wherein

Ring A is chosen from optionally substituted aryls, optionally substituted heteroaryls, optionally substituted cycloalkyls, and optionally substituted heterocycloalkyls;

Ring B is chosen from optionally substituted aryls, optionally substituted heteroaryls, optionally substituted cycloalkyls, and optionally substituted heterocycloalkyls; and

L is chosen from a bond and —NT¹-C(O)—, wherein T¹ is H or Me.

8. A method of inhibiting tumor cell invasion or metastasis mediated by AhR signaling in a subject in need thereof comprising administering to the subject a therapeutically effective amount of a pharmaceutical composition according to embodiment 3 or a therapeutically effective amount of at least one entity chosen from compounds of any one of embodiments 1 and 2, compounds of Formula I

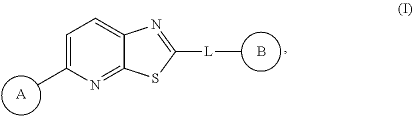

compounds of formula (II)

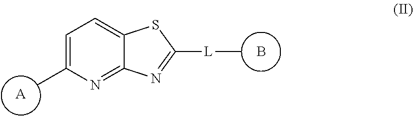

with the proviso that the compound is not

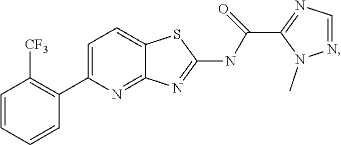 [SMILES: Cn1ncnc1C(=O)Nc1nc2nc(-c3ccccc3C(F)(F)F)ccc2s1]

compounds of formula (III)

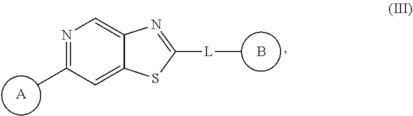

compounds of formula (IV)

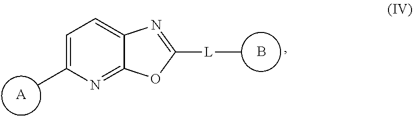

compounds of formula (V)

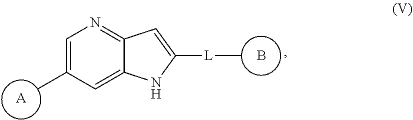

compounds of formula (VI)

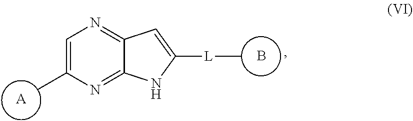

compounds of formula (VII)

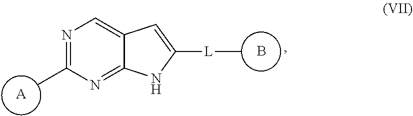

and pharmaceutically acceptable salts thereof,
wherein

Ring A is chosen from optionally substituted aryls, optionally substituted heteroaryls, optionally substituted cycloalkyls, and optionally substituted heterocycloalkyls;

Ring B is chosen from optionally substituted aryls, optionally substituted heteroaryls, optionally substituted cycloalkyls, and optionally substituted heterocycloalkyls; and

L is chosen from a bond and —NT¹-C(O)—, wherein T¹ is H or Me.

9. The method according to any one of embodiments 4-8, wherein the at least one entity is chosen from compounds of any one of embodiments 1 and 2 and pharmaceutically acceptable salts thereof.
10. The method according to any one of embodiments 4-8, wherein the least one entity is chosen from:
1-Methyl-N-(5-(2-(trifluoromethyl)phenyl)thiazolo[5,4-b]pyridin-2-yl)-1H-pyrazole-5-carboxamide;
1-Methyl-N-(5-(2-(trifluoromethyl)phenyl)thiazolo[5,4-b]pyridin-2-yl)-1H-1,2,4-triazole-5-carboxamide;
1-Methyl-N-(5-(o-tolyl)thiazolo[5,4-b]pyridin-2-yl)-1H-1,2,4-triazole-5-carboxamide;
N-(5-(2-(difluoromethyl)phenyl)thiazolo[5,4-b]pyridin-2-yl)-1-methyl-1H-pyrazole-5-carboxamide;
N-(5-(2-(difluoromethyl)phenyl)thiazolo[5,4-b]pyridin-2-yl)-1-methyl-1H-1,2,4-triazole-5-carboxamide;
N-(5-(o-tolyl)thiazolo[5,4-b]pyridin-2-yl)picolinamide;
N-(5-(o-tolyl)thiazolo[5,4-b]pyridin-2-yl)nicotinamide;
N-(5-(o-tolyl)thiazolo[5,4-b]pyridin-2-yl)isonicotinamide;
N-(5-(2-methoxyphenyl)thiazolo[5,4-b]pyridin-2-yl)-1-methyl-1H-1,2,4-triazole-5-carboxamide;
N-(5-(2-(difluoromethyl)-5-fluorophenyl)thiazolo[5,4-b]pyridin-2-yl)-1-methyl-1H-pyrazole-5-carboxamide;
N-(5-(2-(difluoromethyl)-5-fluorophenyl)thiazolo[5,4-b]pyridin-2-yl)-1-methyl-1H-1,2,4-triazole-5-carboxamide;
N-(5-(2-methoxyphenyl)thiazolo[5,4-b]pyridin-2-yl)picolinamide;
N-(5-(2-methoxyphenyl)thiazolo[5,4-b]pyridin-2-yl)nicotinamide;
N-(5-(2-methoxyphenyl)thiazolo[5,4-b]pyridin-2-yl)isonicotinamide;
1-Methyl-N-(5-(o-tolyl)thiazolo[5,4-b]pyridin-2-yl)-1H-pyrazole-5-carboxamide;
N-(5-(3-methoxyphenyl)thiazolo[5,4-b]pyridin-2-yl)-1-methyl-1H-pyrazole-5-carboxamide;
N-(5-(3-(dimethylcarbamoyl)phenyl)thiazolo[5,4-b]pyridin-2-yl)-1-methyl-1H-pyrazole-5-carboxamide;
N-(5-(2-(dimethylamino)phenyl)thiazolo[5,4-b]pyridin-2-yl)-1-methyl-1H-pyrazole-5-carboxamide;
N-(5-(3-(dimethylamino)phenyl)thiazolo[5,4-b]pyridin-2-yl)-1-methyl-1H-pyrazole-5-carboxamide;
N-(5-(4-(dimethylamino)phenyl)thiazolo[5,4-b]pyridin-2-yl)-1-methyl-1H-pyrazole-5-carboxamide;
1-Methyl-N-(5-(2-(pyrrolidin-1-yl)phenyl)thiazolo[5,4-b]pyridin-2-yl)-1H-pyrazole-5-carboxamide;
1-Methyl-N-(5-(1-methyl-1H-pyrazol-4-yl)thiazolo[5,4-b]pyridin-2-yl)-1H-pyrazole-5-carboxamide;
N-(5-(2-(dimethylamino)phenyl)thiazolo[5,4-b]pyridin-2-yl)acetamide;
N-(5-(2-hydroxyphenyl)thiazolo[5,4-b]pyridin-2-yl)-1-methyl-1H-pyrazole-5-carboxamide;
N-(5-(2-methoxyphenyl)thiazolo[5,4-b]pyridin-2-yl)-1-methyl-1H-pyrazole-5-carboxamide;
1-Methyl-N-(5-(pyridin-3-yl)thiazolo[5,4-b]pyridin-2-yl)-1H-pyrazole-5-carboxamide;
1-Methyl-N-(5-(pyridin-2-yl)thiazolo[5,4-b]pyridin-2-yl)-1H-pyrazole-5-carboxamide;
1-Methyl-N-(5-(thiazol-2-yl)thiazolo[5,4-b]pyridin-2-yl)-1H-pyrazole-5-carboxamide;
N-(5-(4,5-dimethylthiazol-2-yl)thiazolo[5,4-b]pyridin-2-yl)-1-methyl-1H-pyrazole-5-carboxamide;
1-Methyl-N-(5-(5-methylthiazol-2-yl)thiazolo[5,4-b]pyridin-2-yl)-1H-pyrazole-5-carboxamide;
1-Methyl-N-(5-(4-methylthiazol-5-yl)thiazolo[5,4-b]pyridin-2-yl)-1H-pyrazole-5-carboxamide;
1-Methyl-N-(5-(4-methylthiazol-2-yl)thiazolo[5,4-b]pyridin-2-yl)-1H-pyrazole-5-carboxamide;
N-(5-(2,4-dimethylthiazol-5-yl)thiazolo[5,4-b]pyridin-2-yl)-1-methyl-1H-pyrazole-5-carboxamide;
N-(5-(2-isobutyramidophenyl)thiazolo[5,4-b]pyridin-2-yl)-1-methyl-1H-pyrazole-5-carboxamide;
N-(5-(2-acetamidophenyl)thiazolo[5,4-b]pyridin-2-yl)-1-methyl-1H-pyrazole-5-carboxamide;
1-Methyl-N-(5-(2-(N-methylacetamido)phenyl)thiazolo[5,4-b]pyridin-2-yl)-1H-pyrazole-5-carboxamide;
N-(5-(2-(2-hydroxyethyl)(methyl)amino)phenyl)thiazolo[5,4-b]pyridin-2-yl)-1-methyl-1H-pyrazole-5-carboxamide;
N-Methyl-N-(5-(o-tolyl)thiazolo[5,4-b]pyridin-2-yl)picolinamide;
N-(5-(o-tolyl)thiazolo[5,4-b]pyridin-2-yl)morpholine-4-carboxamide;
4-Methyl-N-(5-(o-tolyl)thiazolo[5,4-b]pyridin-2-yl)piperazine-1-carboxamide; and
pharmaceutically acceptable salts of any of the foregoing.
   11. The method according to any one of embodiments 4-8, wherein Ring A is chosen from optionally substituted 6-10 membered aryls, optionally substituted 5-10 membered heteroaryls, optionally substituted 3-10 membered cycloalkyls, and optionally substituted 3-10 membered heterocycloalkyls.
   12. The method according to any one of embodiments 4-11, wherein Ring A is chosen from phenyl, pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, and pyrimidinyl, each of which may be substituted with one or more substituents, which may be the same or different.
   13. The method according to any one of embodiments 4-11, wherein Ring A is chosen from

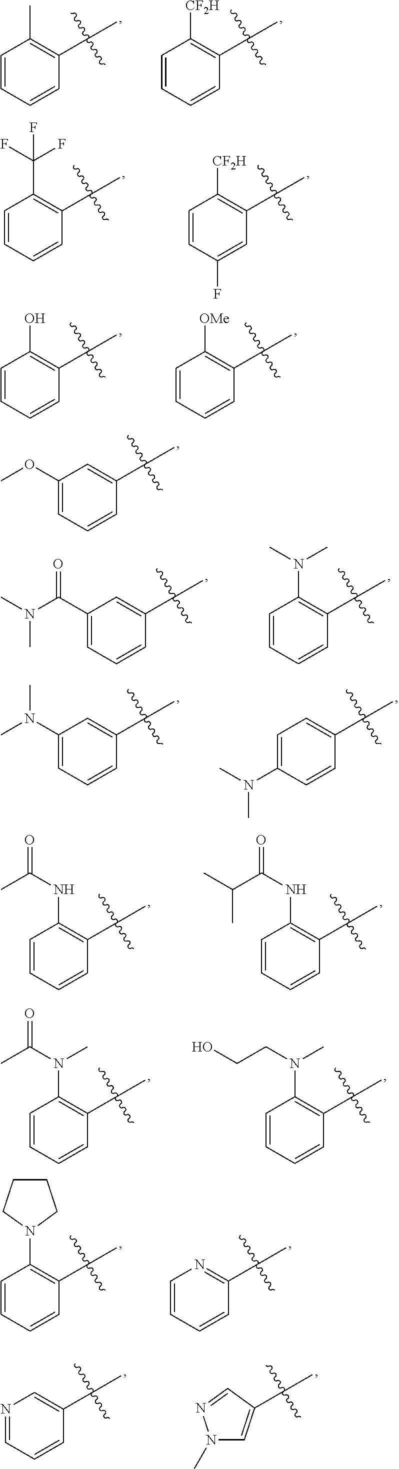

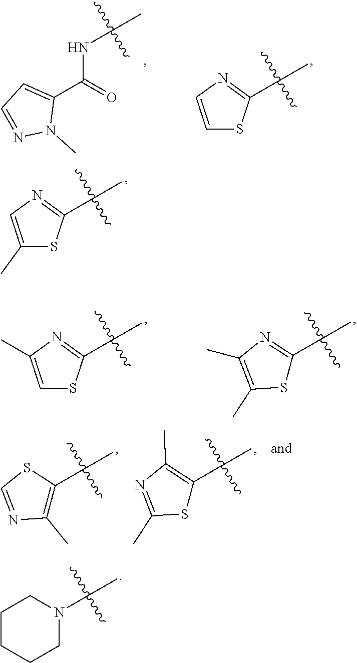

14. The method according to any one of embodiments 4-13, wherein Ring B is chosen from optionally substituted 6-10 membered aryls, optionally substituted 5-10 membered heteroaryls, optionally substituted 3-10 membered cycloalkyls, and optionally substituted 3-10 membered heterocycloalkyls.
15. The method according to any one of embodiments 4-14, wherein Ring B is chosen from phenyl, pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, pyridinonyl, pyrimidinyl, piperidinyl, piperazinyl, and morpholinyl each of which may be substituted with one or more substituents, which may be the same or different.
16. The method according to any one of embodiments 4-14, wherein Ring B is chosen from

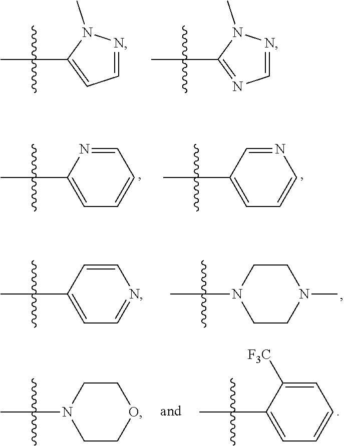

17. The method according to any one of embodiments 4-16, wherein L is a bond, —NH(C═O)—, or —NCH₃(C═O)—.
18. The method of any one of embodiments 4-17, wherein the disease is chosen from cancers.
19. The method of any one of embodiments 4-18, wherein the disease is chosen from breast cancers, respiratory tract cancers, brain cancers, cancers of reproductive organs, digestive tract cancers, urinary tract cancers, eye cancers, liver cancers, skin cancers, head and neck cancers, thyroid cancers, parathyroid cancers, and metastases of any of the foregoing.
20. The method of any one of embodiments 4-18, wherein the disease is chosen from lymphomas, sarcomas, melanomas, glioblastomas, and leukemias.
21. The method of any one of embodiments 4 to 20, further comprising administering to the subject a therapeutically effective amount of at least one immune checkpoint inhibitor.
22. The method of embodiment 21, wherein the immune checkpoint inhibitor is chosen from PD-1 inhibitors, PD-L1 inhibitors, and CTLA-4 blockers.

### EXAMPLES

The following non-limiting examples and data illustrate various aspects and features relating to the compounds and/or methods of the present disclosure, including the preparation of various compounds, as are available through the synthetic methodologies described herein. In comparison with the prior art, in some embodiments, the present compounds and/or methods provide results and data which are surprising, unexpected and contrary thereto. While the utility of this disclosure is illustrated through the use of several compounds and moieties/groups which can be used therewith, it will be understood by those skilled in the art that comparable results are obtainable with various other compounds, moieties and/or groups, as are commensurate with the scope of this disclosure.

### Example 1: Preparation of 1-methyl-N-(5-(2-(trifluoromethyl)phenyl)thiazolo[5,4-b]pyridin-2-yl)-1H-pyrazole-5-carboxamide (Product 1)

### Step 1. Preparation of 5-bromothiazolo[5,4-b]pyridin-2-amine (2)

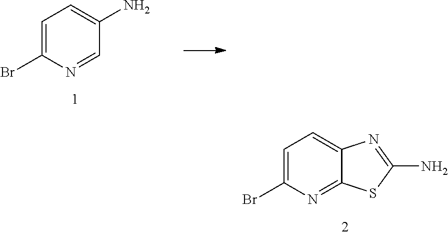 [SMILES: Nc1ccc(Br)nc1 ; Nc1nc2ccc(Br)nc2s1]

6-Bromopyridin-3-amine (1, 15 g, 86.70 mmol) was added to a mixture of potassium thiocyanate (42.13 g, 433.50 mmol) in HOAc (200 mL). A mixture of Bra (9.4 M, 11.99 mL) in 200 mL HOAc was added dropwise at 0° C. to the reaction mixture that was subsequently degassed and purged with N₂ for three times. The mixture was stirred at 25° C. for 12 hours under N₂ atmosphere. The reaction mixture was filtered; the filtrate was concentrated and the crude reaction mixture was adjusted to pH=7 with saturated NaHCO₃ aqueous. The mixture was diluted with EtOAc (200 mL) and washed three times with water (200 mL), dried with anhydrous sodium sulfate, filtered and concentrated under reduced pressure to provide the crude material. The residue was purified by column chromatography to provide the free base of the title compound (2, 10 g, 50% yield). ¹H-NMR (DMSO-d₆, 400 MHz): δH 7.95 (2H, s), 7.56 (1H, d, J=8.4 Hz), 7.41 (1H, d, J=8.4 Hz); MS (m/z):231.9 [M+H]⁺.

### Step 2. Preparation of tert-butyl (5-bromothiazolo[5,4-b]pyridin-2-yl)carbamate (3)

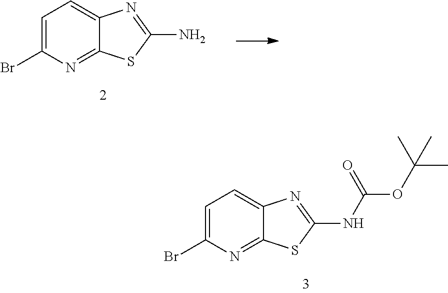 [SMILES: Nc1nc2ccc(Br)nc2s1 ; CC(C)(C)OC(=O)Nc1nc2ccc(Br)nc2s1]

Triethylamine (2.86 g, 28.25 mmol, 3.93 mL) was added to a mixture of 5-bromothiazolo[5,4-b]pyridin-2-amine (2, 5.0 g, 21.73 mmol), di-tert-butyl dicarbonate (6.17 g, 28.25 mmol, 6.49 mL) and DMAP (265.49 mg, 2.17 mmol) in THF (150 mL). The reaction mixture was degassed, purged with N₂ for 3 times and stirred at 25° C. for 5 hours under N₂ atmosphere. The solvent was removed by reduced pressure. The residue was taken up in ethyl acetate (20 mL), washed three times with water (100 mL), three times with a 0.06M HCl solution (100 mL), three times with brine (100 mL) and dried over anhydrous sodium sulfate.

The solution was filtered and the solvent was removed under reduced pressure to afford the crude product. The crude product was washed with a mixture of petroleum ether (100 mL) and ethyl acetate (50 mL). The mixture was filtered and the filter cake was dried under reduced pressure to provide the free base of the title compound (3, 6 g, 42% yield). ¹H-NMR (DMSO-d₆, 400 MHz): δH 12.10 (1H, s), 7.99 (1H, d, J=8.4 Hz), 7.63 (1H, d, J=8.4 Hz), 1.52 (9H, s); MS (m/z):331.9 [M+H]⁺.

### Step 3. Preparation of tert-butyl (5-(2-(trifluoromethyl)phenyl)thiazolo[5,4-b]pyridin-2-yl)carbamate (5)

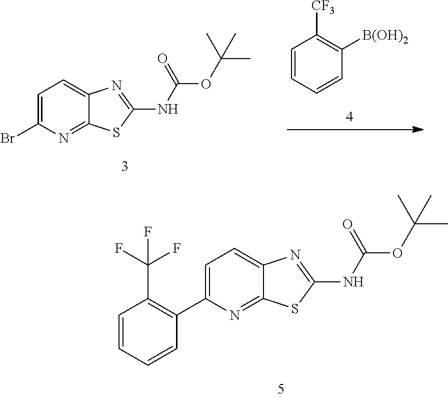

Bis(triphenylphosphine)palladium(II)dichloride (Pd(PPh₃)₂Cl₂, 1.06 g, 1.51 mmol, 0.2 eq) was added to a mixture of tert-butyl (5-bromothiazolo[5,4-b]pyridin-2-yl)carbamate (3, 2.5 g, 7.57 mmol), (2-(trifluoromethyl)phenyl)boronic acid (4, 3.45 g, 18.17 mmol), Cs₂CO₃ (7.40 g, 22.71 mmol) in DMF (60 mL) and water (16 mL). The mixture was degassed, purged with N₂ for three times and stirred at 120° C. for 10 hours under N₂ atmosphere. The reaction mixture was concentrated under reduced pressure and the residue was purified by column chromatography to provide the free base of the title compound (5, 580 mg, 19% yield). ¹H-NMR (DMSO-d₆, 400 MHz): δH 12.03 (1H, s), 8.12 (1H, d, J=8.4 Hz), 7.87 (1H, d, J=8.0 Hz), 7.77 (1H, t, J=7.2), 7.68 (1H, t, J=7.6), 7.61 (1H, d, J=7.6), 7.54 (1H, d, J=8.4), 1.53 (9H, s); MS (m/z):396.0 [M+H]⁺.

Intermediates A reported in Table 1 were prepared according to the above step:

TABLE 1

| | | | MS (m/z) |
|---|---|---|---|
| | | | [M + H]⁺; |
| Intermediate name | Structure and name | ¹H-NMR | Purity (%) |
| 1A (for Product 2) | 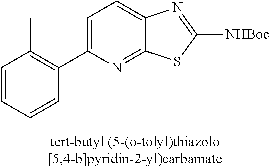 [SMILES: Cc1ccccc1-c1ccc2nc(NC(=O)OC(C)(C)C)sc2n1] | (DMSO-d₆, 400 MHz): δH 11.97 (1H, s), 8.09 (1H, d, J = 8.4 Hz), 7.56 (1H, d, J = 8.4 Hz), 7.43 (1H, d, J = 7.2 Hz), 7.30-7.33 (3H, m), 2.34 (3H, s), 1.53 (9H, s) | 342.1; NA |
| 2A (for Product 3) | 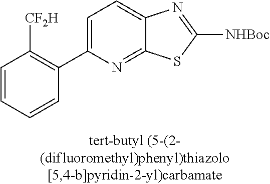 [SMILES: CC(C)(C)OC(=O)Nc1nc2ccc(-c3ccccc3C(F)F)nc2s1] | (CDCl3-d1, 400 Hz): δH 9.53 (1H, s), 8.12 (1H, d, J = 8.4 Hz), 7.85 (1H, d, J = 6.8 Hz), 7.55-7.60 (4H, m), 7.16 (1H, t, J = 55.6 Hz), 1.25 (9H, s). | NA; NA |
| 3A (for Product 4)* | 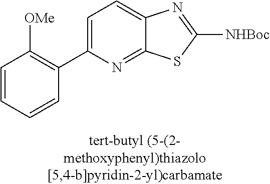 [SMILES: COc1ccccc1-c1ccc2nc(NC(=O)OC(C)(C)C)sc2n1] | (400 MHz, DMSO-d6) δ H 1.53 (s, 9H), 3.84 (s, 3H), 7.08 (t, J = 7.6 Hz, 1H), 7.17 (d, J = 8.0 Hz, 1H), 7.36-7.46 (m, 1H), 7.75 (dd, J = 2.0 Hz, J = 8.0 Hz, 1H), 7.88 (d, J = 8.8 Hz, 1H), 8.03 (d, J = 8.4 Hz, 1H), 11.93 (s, 1H). | 358.0; NA |
| 4A (for Product 5) | 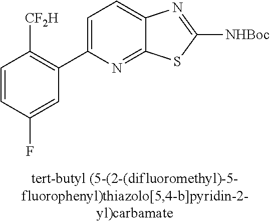 [SMILES: CC(C)(C)OC(=O)Nc1nc2ccc(-c3cc(F)ccc3C(F)F)nc2s1] | (DMSO-d6, 400 MHz): δH 12.08 (1H, s), 8.17 (1H, d, J = 8.4 Hz), 7.84 (1H, t, J = 6.0 Hz), 7.74 (1H, d, J = 8.4 Hz), 7.56 (1H, d, J = 9.6 Hz), 7.46 (1H, s), 7.32 (1H, t, J = 55.2 Hz), 1.53 (9H, s). | 396.0; NA |

*Dioxane, K₂CO₃ and Pd(dppf)Cl₂•CH₂Cl₂ were used instead of water, CsCO₃ and Pd(PPh₃)₂Cl₂.

### Step 4. Preparation of 5-(2-(trifluoromethyl)phenyl)thiazolo[5,4-b]pyridin-2-amine (6)

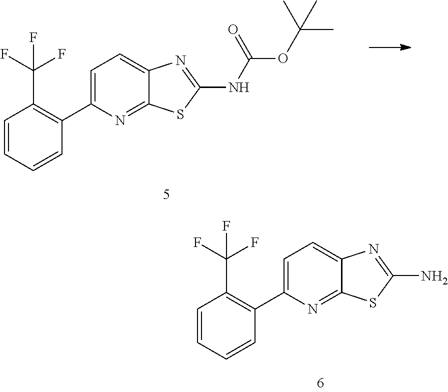 [SMILES: CC(C)(C)OC(=O)Nc1nc2ccc(-c3ccccc3C(F)(F)F)nc2s1 ; Nc1nc2ccc(-c3ccccc3C(F)(F)F)nc2s1]

Trifluoroacetic acid (20 mL) was added at 0° C. to a mixture of tert-butyl (5-(2-(trifluoromethyl)phenyl)thiazolo[5,4-b]pyridin-2-yl)carbamate (5, 580 mg, 1.47 mmol) in DCM (20 mL). The reaction mixture was warmed to 25° C., degassed and purged with N₂ for three times. The mixture was stirred at 25° C. for 2 hours under N₂ atmosphere. The reaction mixture was concentrated under reduced pressure. An aqueous saturated solution of NaHCO₃ (30 mL) was added to the residue. The mixture was filtered and the filter cake was dried under reduced pressure to provide the free base of the title compound (6, 300 mg, 69% yield). ¹H-NMR (DMSO-d₆, 400 MHz): δH 7.90 (2H,$), 7.84 (1H, d, J=8.0 Hz), 7.74 (1H, t, J=7.2 Hz), 7.69 (1H, d, J=8.0 Hz), 7.64 (1H, t, J=7.6 Hz), 7.56 (1H, d, J=7.2 Hz), 7.33 (1H, d, J=8.4 Hz); MS (m/z):296.0 [M+H]⁺.

Intermediates B Reported in Table 2 were Prepared According to the Above Step:

TABLE 2

| | | | MS (m/z) |
|---|---|---|---|
| | | | [M + H]⁺; |
| Intermediate name | Structure and name | ¹H-NMR | Purity (%) |
| 1B (for Product 2) | 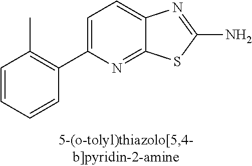 [SMILES: Cc1ccccc1-c1ccc2nc(N)sc2n1] | (DMSO-d₆, 400 MHz): δH 7.82 (2H, s), 7.67 (1H, d, J = 8.0 Hz), 7.34-7.39 (2H, m), 7.25-7.29 (3H, m), 2.33 (3H, s). | 242.0; NA |
| 2B (for Product 3) | 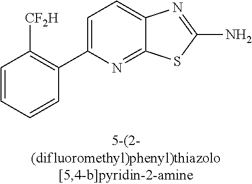 [SMILES: Nc1nc2ccc(-c3ccccc3C(F)F)nc2s1] | | NA; NA |
| 3B (for Product 4 + others) | 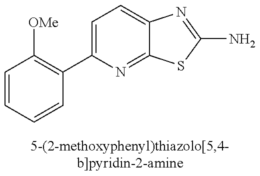 [SMILES: COc1ccccc1-c1ccc2nc(N)sc2n1] | (400 MHz, DMSO-d6) δ H 3.83 (s, 3H), 7.04 (t, J = 7.2 Hz, 1H), 7.13 (d, J = 8.4 Hz, 1H), 7.34-7.43 (m, 1H), 7.62 (d, J = 8.4 Hz, 1H), 7.65-7.75 (m, 2H), 7.81 (s, 2H). | NA; NA |
| 4B (for KProduct 5) | 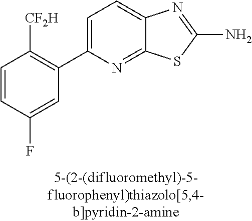 [SMILES: Nc1nc2ccc(-c3cc(F)ccc3C(F)F)nc2s1] | NA | NA |

### Step 5. Preparation of 1-methyl-1H-pyrazole-5-carbonyl chloride (8)

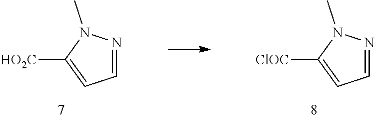

Two drops of DMF (3.19 mg, 43.61 mmol, 3.36 mL) were added to a solution of 1-methyl-1H-pyrazole-5-carboxylic acid (7, 110 mg, 872.23 mmol) and SOCl₂ (207.54 mg, 1.74 mmol, 126.55 mL) in DCM (5.0 mL). The mixture was degassed and purged with N₂ for 3 times and stirred at 40° C. for 2 hours under N₂ atmosphere. After addition of methylbenzene (5.0 mL), the reaction mixture was concentrated under reduced pressure to provide the free base of the title compound (8, 100 mg, 79% yield) that was used in the next step without any further purification. MS (m/z): 196.1 [M+H]⁺.

### Step 6. Preparation of 1-methyl-N-(5-(2-(trifluoromethyl)phenyl)thiazolo[5,4-b]pyridin-2-yl)-1H-pyrazole-5-carboxamide (Product 1)

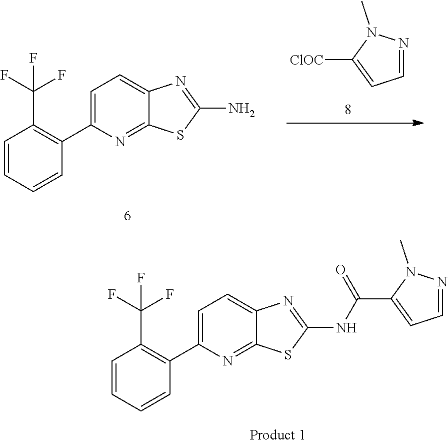

Triethylamine (TEA, 34.27 mg, 338.66 mmol, 47.14 mL) was added to a mixture of 5-(2-(trifluoromethyl)phenyl)thiazolo[5,4-b]pyridin-2-amine (6, 100 mg, 338.66 mmol), 1-methyl-1H-pyrazole-5-carbonyl chloride (8, 97.91 mg, 677.32 mmol) and DMAP (41.37 mg, 338.66 mmol) in DCM (10 mL). The reaction mixture was degassed, purged with N₂ for three times and stirred at 25° C. for 8 hours under N₂ atmosphere. The solvent was removed under reduced pressure and the residue was taken up in ethyl acetate (20 mL), washed three times with brine (10 mL) and dried over anhydrous sodium sulfate. The organic phase was filtered and concentrated. The residue was purified by silica-gel prep-TLC to provide the free base of the title compound (Product 1, 58 mg, 42% yield). ¹H-NMR (DMSO-d₆, 400 MHz): δH 13.09 (1H, s), 8.24 (1H, d, J=8.4 Hz), 7.89 (1H, d, J=7.6 Hz), 7.79 (1H, t, J=7.6 Hz), 7.70 (1H, t, J=7.6 Hz), 7.61-7.65 (3H, m), 7.44 (1H, d, J=2.4 Hz), 4.17 (3H, s); MS (m/z): 404.0 [M+H]⁺; purity 98%.

Final compounds reported in Table 3 were prepared according to step 6 for the synthesis of Product 1, with minor modifications as reported in the notes.

TABLE 3

| | | | MS |
|---|---|---|---|
| | | | (m/z) |
| | | | [M + H]⁺; |
| Final | | | Purity |
| compound | Structure and name | ¹H-NMR | (%) |
| Product 6 | 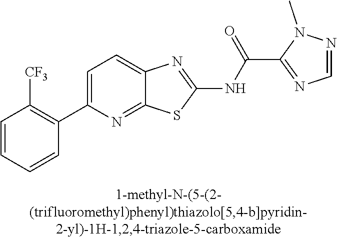 [SMILES: Cn1ncnc1C(=O)Nc1nc2ccc(-c3ccccc3C(F)(F)F)nc2s1] | (DMSO-d₆, 400 MHz): δH 13.25 (1H, s), 8.29 (1H, d, J = 8.0 Hz), 8.25 (1H, s), 7.89 (1H, d, J = 8.0 Hz), 7.80 (1H, t, J = 7.2 Hz), 7.71 (1H, t, J = 7.6 Hz), 7.63-7.66 (2H, m), 4.22 (3H, s). | 405.0; 99.9 |
| Product 2^{b} | 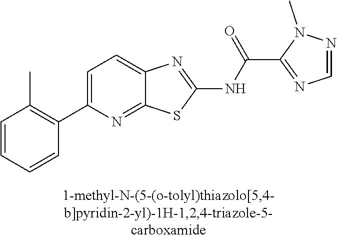 [SMILES: Cc1ccccc1-c1ccc2nc(NC(=O)c3ncnn3C)sc2n1] | (DMSO-d6, 400 MHz): δH 7.88 (1H, s), 7.82 (1H, d, J = 8.4 Hz), 7.42 (1H, t, J = 4.4 Hz), 7.37 (1H, d, J = 8.0 Hz), 7.27-7.30 (3H, m), 6.33 (1H, s) 4.22 (3H, s), 2.36 (3H, s). | 351.2; 94.0 |
| Product 3^{c} | 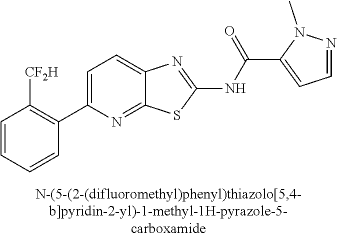 [SMILES: Cn1nccc1C(=O)Nc1nc2ccc(-c3ccccc3C(F)F)nc2s1] | (DMSO-d6, 400 MHz): δH 13.09 (1H, s), 8.28 (1H, d, J = 8.4 Hz), 7.79 (2H, dd, J = 7.2 Hz, J = 14.0 Hz), 7.62-7.72 (4H, m), 7.45 (1H, s), 7.37 (1H, t, J = 15.2 Hz), 4.18 (3H, s). | 386.1; 98.0 |
| Product 7^{c} | 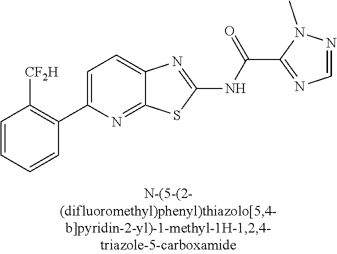 [SMILES: Cn1ncnc1C(=O)Nc1nc2ccc(-c3ccccc3C(F)F)nc2s1] | (DMSO-d6, 400 MHz): δH 13.28 (1H, s), 8.32 (1H, d, J = 8.4 Hz), 8.25 (1H, s), 7.80 (2H, t, J = 10.0 Hz), 7.61-7.75 (3H, m), 7.38 (1H, t, J = 54.8 Hz), 4.21 (3H, s). | 386.9, 94.0 |
| Product 8^{d} | 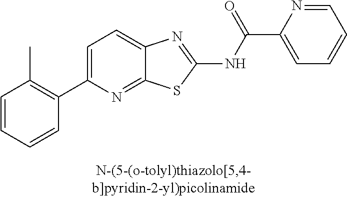 [SMILES: Cc1ccccc1-c1ccc2nc(NC(=O)c3ccccn3)sc2n1] | (DMSO-d6, 400 MHz): δH 13.25 (1H, s), 8.29 (1H, d, J = 8.0 Hz), 8.25 (1H, s), 7.89 (1H, d, J = 8.0 Hz), 7.80 (1H, t, J = 7.2 Hz), 7.71 (1H, t, J = 7.6 Hz), 7.63-7.66 (2H, m), 4.22 (3H, s). | 405.0, 99.9 |
| Product 9^{d} | 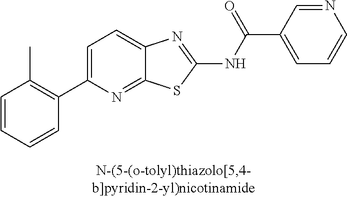 [SMILES: Cc1ccccc1-c1ccc2nc(NC(=O)c3cccnc3)sc2n1] | (400 MHz, DMSO-d6): δ H 2.38 (s, 3H), 7.31-7.36 (m, 3H), 7.47 (d, J = 6.8 Hz, 1H), 7.61-7.67 (m, 2H), 8.21 (d, J = 8.4 Hz, 1H), 8.47-8.50 (m, 1H), 8.82-8.84 (m, 1H), 9.27 (d, J = 1.6 Hz, 1H), 13.27 (s, 1H). | 347.1; 98.0 |
| Product 10^{d} | 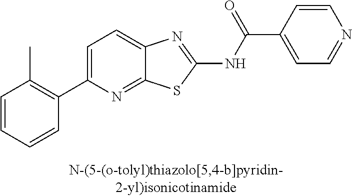 [SMILES: Cc1ccccc1-c1ccc2nc(NC(=O)c3ccncc3)sc2n1] | (400 MHz, DMSO-d6) δH 2.37 (s, 3H), 7.27-7.36 (m, 3H), 7.45 (d, J = 6.4 Hz, 1H), 7.58 (d, J = 8.0 Hz, 1H), 8.04 (d, J = 4.8 Hz, 2H), 8.12 (d, J = 7.2 Hz, 1H), 8.80 (d, J = 4.4 Hz, 2H), 13.41 (s, 1 H). | 347.0; 99.0 |
| Product 4^{c} | 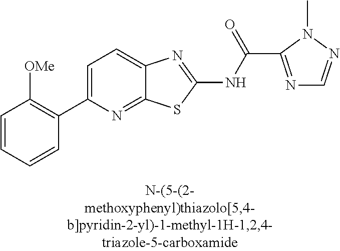 [SMILES: COc1ccccc1-c1ccc2nc(NC(=O)c3ncnn3C)sc2n1] | (400 MHz, DMSO-d6) δH 3.86 (s, 3H), 4.22 (s, 3H), 7.10 (t, J = 7.6 Hz, 1H), 7.19 (d, J = 7.6 Hz, 1H), 7.44 (t, J = 7.2 Hz, 1H), 7.75-7.83 (m, 1H), 7.98 (d, J = 8.4 Hz, 1H), 8.19 (d, J = 8.4 Hz, 1H), 8.24 (s, 1H), 13.16 (s, 1H). | 367.0; 97.0 |
| Product 5^{c} | 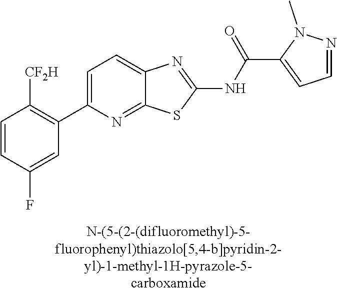 [SMILES: Cn1nccc1C(=O)Nc1nc2ccc(-c3cc(F)ccc3C(F)F)nc2s1] | (DMSO-d6, 400 MHz) δH 13.12 (1H, s), 8.29 (1H, d, J = 8.4 Hz), 7.87 (1H, dd, J = 6.0 Hz, J = 8.8 Hz), 7.82 (1H, d, J = 8.4 Hz), 7.58-7.62 (2H, m), 7.45-7.51 (2H, m), 7.36 (1H, t, J = 55.2 Hz), 4.17 (3H, s) | 404.1, 98 |
| Product 11^{c} | 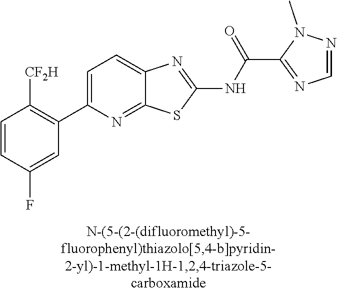 [SMILES: Cn1ncnc1C(=O)Nc1nc2ccc(-c3cc(F)ccc3C(F)F)nc2s1] | (400 MHz, DMSO-d6) δ ppm 13.21 (s, 1H), 8.25 (d, J = 8.4 Hz, 1H), 8.19 (s, 1H), 7.86 (q, J = 5.6 Hz, J = 8.8 Hz, 1H), 7.79 (d, J = 8.4 Hz, 1H), 7.58 (d, J = 10.0 Hz, 1H), 7.23-7.50 (m, 2H), 4.22 (s, 3H); MS (m/z): 405.0 [M + H]+; 99% purity | 405.0; 99.0 |
| Product 12^{c} | 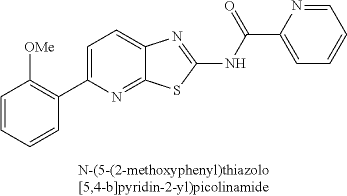 [SMILES: COc1ccccc1-c1ccc2nc(NC(=O)c3ccccn3)sc2n1] | (400 MHz, DMSO-d6) δ H 3.86 (s, 3H), 7.10 (t, J = 7.6 Hz, 1H), 7.19 (d, J = 8.4 Hz, 1H), 7.44 (t, J = 7.2 Hz, 1H), 7.76- 7.81 (m, 2H), 7.98 (d, J = 8.4 Hz, 1H), 8.10-8.28 (m, 3H), 8.82 (d, J = 4.4 Hz, 1H), 12.41 (s, 1H). | 363.1; 95.0 |
| Product 13^{c} | 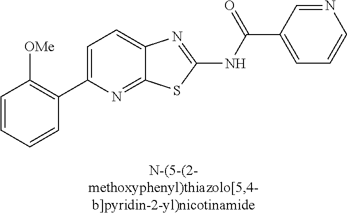 [SMILES: COc1ccccc1-c1ccc2nc(NC(=O)c3cccnc3)sc2n1] | (400 MHz, DMSO-d6): δ H 3.86 (s, 3H), 7.10 (d, J = 7.6 Hz, 1H), 7.19 (d, J = 8.4 Hz, 1H), 7.42-7.46 (m, 1H), 7.62 (dd, J = 4.8 Hz, J = 7.6 Hz, 1H), 7.79 (dd, J = 1.2 Hz, J = 7.6 Hz, 1H), 7.98 (d, J = 8.4 Hz, 1H), 8.17 (d, J = 8.4 Hz, 1H), 8.47 (d, J = 8.0 Hz, 1H), 8.80-8.85 (m, 1H), 9.26 (d, J = 2.0 Hz, 1H), 13.23 (s, 1H). | 363.0; 99.0 |
| Product 14^{c} | 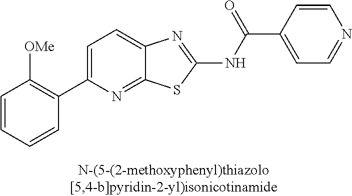 [SMILES: COc1ccccc1-c1ccc2nc(NC(=O)c3ccncc3)sc2n1] | (400 MHz, DMSO-d6): δ H 3.86 (s, 3H), 7.10 (d, J = 7.6 Hz, 1H), 7.19 (d, J = 8.4 Hz, 1H), 7.42-7.46 (m, 1H), 7.79 (dd, J = 1.6 Hz, J = 7.6 Hz, 1H), 7.99 (d, J = 8.4 Hz, 1H), 8.03 (d, J = 6.0 Hz, 2H), 8.18 (d, J = 8.4 Hz, 1H), 8.85 (d, J = 6.0 Hz, 2H), 13.33 (s, 1H). | 363.0; 97.0 |
| Product 15^{a} | 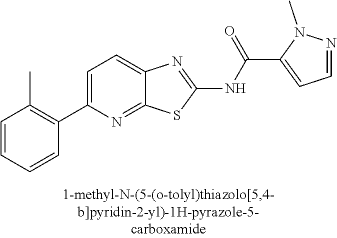 [SMILES: Cc1ccccc1-c1ccc2nc(NC(=O)c3ccnn3C)sc2n1] | (DMSO-d6, 400 MHz): δH 13.05 (1H, s), 8.19 (1H, d, J = 8 Hz), 7.64 (1H, d, J = 8.4 Hz), 7.60 (1H, d, J = 1.6 Hz), 7.47 (1H, d, J = 6.4 Hz), 7.41 (1H, s), 7.34 (3H, s), 4.17 (3H, s), 2.37 (3H, s). | 350.0; 97.0 |

^{a}A solution of acyl chloride in THF was added to a solution of all the other reagents. The reaction mixture was stirred at 60° C. for 8 hours.
^{b}TEA was added to a solution of all the other reagents in DCM. The reaction mixture was stirred at 60° C. for 8 hours.
^{c}A solution of TEA, DMAP and acyl chloride in THF was added to a solution of amine in THF at 0° C. The reaction mixture was stirred at room temperature until completion of the reaction.
^{d}Acetyl chloride was added at 0° C. to a solution of all the reagents in DCM. The reaction mixture was stirred at room temperature until completion of the reaction.

### Example 2: Preparation of 1-methyl-1H-1,2,4-triazole-5-carbonyl chloride (10)

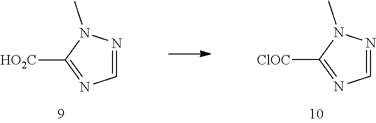

Two drops of DMF (28.75 mg, 393.39 mmol, 30.27 mL) were added to a solution of 1-methyl-1H-1,2,4-triazole-5-carboxylic acid (9, 1 g, 7.87 mmol) and oxalyl chloride (2.00 g, 15.74 mmol, 1.38 mL) in DCM (5.0 mL). The mixture was degassed and purged with N₂ for three times and stirred at 40° C. for 1 hour under N₂ atmosphere. The reaction mixture was concentrated under reduced pressure to provide the free base of the title compound (10, 1 g, crude). MS (m/z): 197.1 [M+H]+.

### Example 3: Preparation of 2-(2-(difluoromethyl)phenyl)-4,4,5,5-tetramethyl-1,3,2-dioxaborolane (13)

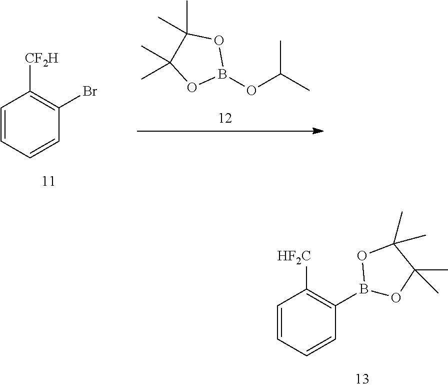

n-Butyllithium (2.5 M, 4.25 mL) was added to a solution of 1-bromo-2-(difluoromethyl) benzene (11, 2.0 g, 9.66 mmol) in THF (20 mL) at −70° C. The reaction mixture was stirred for 1 hour at −70° C. Then 2-isopropoxy-4,4,5,5-tetramethyl-1,3,2-dioxaborolane (12, 2.34 g, 12.56 mmol, 2.56 mL) was added into the mixture at −70° C. and the reaction mixture was stirred for 12 hours at 25° C. Water was added to the mixture (20 mL) at 0° C. The mixture was extracted three times with EtOAc (20 ml). The combined organic layers were dried over anhydrous sodium sulfate and filtered. The solvent was removed under reduced pressure and the remaining residue was purified by silica gel column chromatography to provide the free base of the title compound (13, 1.5 g, 55% yield). ¹H-NMR (CDCl₃-d₁, 400 MHz): δH 7.91 (1H, d, J=7.2 Hz), 7.75 (1H, d, J=7.6 Hz), 7.56 (1H, t, J=7.6 Hz), 7.41-7.51 (2H, m), 1.39 (12H, s).

### Example 4: Preparation of 2-(2-(difluoromethyl)-5-fluorophenyl)-4,4,5,5-tetramethyl-1,3,2-dioxaborolane (16)

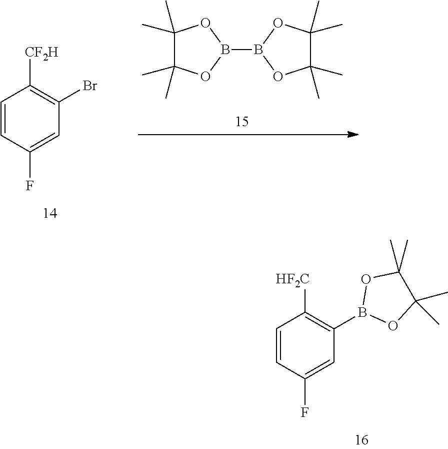

Potassium acetate (1.48 g, 15.11 mmol) and Pd(dppf)Cl₂ (162.60 mg, 222.22 mmol) were added to a solution of 2-bromo-1-(difluoromethyl)-4-fluorobenzene (14, 1.0 g, 4.44 mmol) and 4,4,4′,4′,5,5,5′,5′-octamethyl-2,2′-bi(1,3,2-dioxaborolane) (15, 1.35 g, 5.33 mmol) in dioxane (10 mL) and DMSO (1.0 mL). The reaction mixture was stirred at 85° C. for 3 hours, then it was diluted with water (10 ml) and extracted three times with EtOAc (10 mL). The combined organic layers were washed three times with brine (10 mL), dried over anhydrous sodium sulfate, filtered and concentrated under reduced pressure to give a residue. The residue was purified by silica gel column chromatography to provide the free base of the title compound (16, 450 mg, 33% yield).

### Example 5: Preparation of N-(5-(3-methoxyphenyl)thiazolo[5,4-b]pyridin-2-yl)-1-methyl-1H-pyrazole-5-carboxamide (Product 16)

### Step 1. Preparation of N-(5-bromothiazolo[5,4-b]pyridin-2-yl)-1-methyl-1H-pyrazole-5-carboxamide (17)

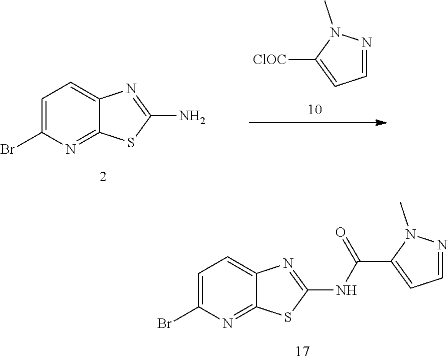

4-Dimethylaminopyridine (1.43 g, 11.73 mmol) and TEA (1.19 g, 11.73 mmol, 1.63 mL) were added to a solution of 1-methyl-1H-pyrazole-5-carbonyl chloride (10, 3.39 g, 23.47 mmol) in DCM (30 mL). Subsequently, 5-bromothiazolo[5,4-b]pyridin-2-amine (2, 2.7 g, 11.73 mmol) was added at 0° C. The mixture was stirred at 25° C. for 5 hours. The reaction mixture was quenched by addition of water (30 mL) at 0° C. The reaction mixture was filtered and the filter cake was dried to provide the free base of the title compound (17, 3.6 g, 91% yield). ¹H-NMR (DMSO-d₆, 400 MHz): δH 13.11 (1H, s), 8.09 (1H, d, J=8.4 Hz), 7.71 (1H, d, J=8.4 Hz), 7.60 (1H, d, J=2.0 Hz), 7.41 (1H, d, J=2.0 Hz), 4.15 (3H, s); MS (m/z): 339.9 [M+H]⁺.

### Step 2. Preparation of N-(5-(3-methoxyphenyl)thiazolo[5,4-b]pyridin-2-yl)-1-methyl pyrazole-5-carboxamide (Product 16)

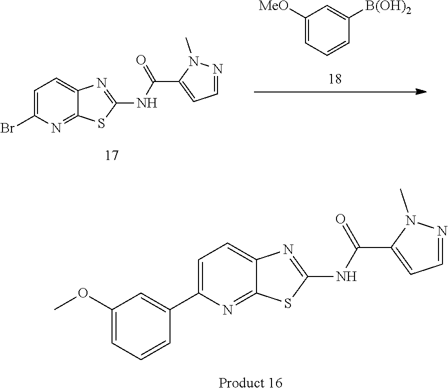 [SMILES: Cn1nccc1C(=O)Nc1nc2ccc(Br)nc2s1 ; COc1cccc(B(O)O)c1 ; COc1cccc(-c2ccc3nc(NC(=O)c4ccnn4C)sc3n2)c1]

Product 16 was prepared according to the procedure reported for step 3 for the synthesis of Product 1 starting from N-(5-bromothiazolo[5,4-b]pyridin-2-yl)-1-methyl-1H-pyrazole-5-carboxamide (17) and (3-methoxyphenyl)boronic acid (18). ¹H-NMR (DMSO-d₆, 400 MHz): δH 7.82 (1H, d, J=8.4 Hz), 7.75 (1H, d, J=8.4 Hz), 7.62-7.65 (2H, m), 7.37 (2H, t, J=8.0 Hz), 6.92-6.95 (1H, m), 6.76 (1H, s), 4.21 (3H, s), 3.85 (3H, s); MS (m/z):366.1 [M+H]⁺; purity 95%.

Final compounds reported in Table 4 were prepared according to step 2 for the synthesis of Product 16 using the corresponding boronic acid.

TABLE 4

| | | | MS |
|---|---|---|---|
| | | | (m/z) |
| | | | [M + |
| Final | | | H]⁺; |
| com- | | | Purity |
| pound | Structure and name | ¹H-NMR | (%) |
| Product 17^{a} | 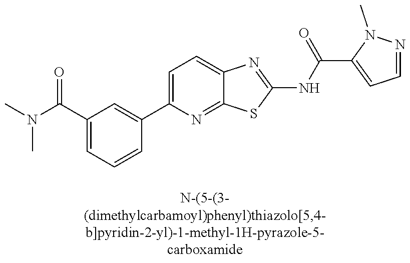 [SMILES: CN(C)C(=O)c1cccc(-c2ccc3nc(NC(=O)c4ccnn4C)sc3n2)c1] | (DMSO-d₆, 400 MHz): δH 8.14 (1H, d, J = 8.0 Hz), 8.08 (1H, s), 7.89 (1H, d, J = 8.4 Hz), 7.80 (1H, d, J = 8.4 Hz), 7.53 (1H, t = 7.6 Hz), 7.36-7.39 (2H, m), 7.14 (1H, s), 6.81 (1H, d, J = 1.6 Hz), 4.20 (3H, s), 2.99 (6H, d, J = 25.2 Hz). | 406.9; 98.0 |
| Product 18 | 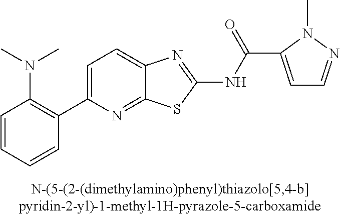 [SMILES: CN(C)c1ccccc1-c1ccc2nc(NC(=O)c3ccnn3C)sc2n1] | (400 MHz, DMSO-d₆) δH 2.54 (s, 6H), 4.18 (s, 3H), 7.06 (t, J = 7.2 Hz, 1H), 7.12 (d, J = 8.0 Hz, 1H), 7.32-7.36 (m, 1H), 7.41 (s, 1H), 7.54 (dd, J = 1.6 Hz, J = 7.6 Hz, 1H), 7.60 (d, J = 2.4 Hz, 1H), 8.00 (d, J = 4.4 Hz, 1H), 8.13 (d, J = 8.4 Hz, 1H), 13.00 (s, 1H). | 379.1; 99.9 |
| Product 19 | 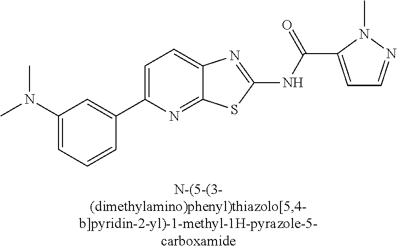 [SMILES: CN(C)c1cccc(-c2ccc3nc(NC(=O)c4ccnn4C)sc3n2)c1] | (DMSO-d₆, 400 MHz): δH 13.04 (1H, s), 8.17 (1H, d, J = 8.4 Hz), 8.07 (1H, d, J = 8.8 Hz), 7.61 (1H, d, J = 2.0 Hz), 7.37-7.47 (3H, m), 7.32 (1H, t, J = 8.4 Hz), 6.82 (1H, dd, J = 2.4 Hz, J = 8.0 Hz), 4.17 (3H, s), 3.00 (6H, s). | 379.0; 98.0 |
| Product 20 | 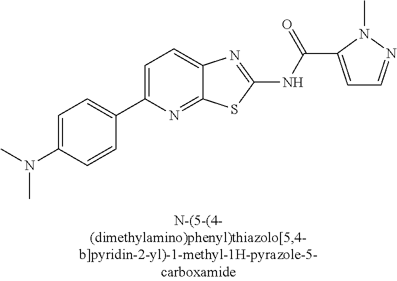 [SMILES: CN(C)c1ccc(-c2ccc3nc(NC(=O)c4ccnn4C)sc3n2)cc1] | (DMSO-d₆, 400 MHz): δH 12.96 (1H, s), 8.09 (1H, d, J = 8.4 Hz), 7.98 (3H, dd, J = 9.2 Hz, J = 19.2 Hz), 7.60 (1H, d, J = 2.0 Hz), 7.42 (1H, brs), 6.82 (2H, d, J = 9.2 Hz), 4.17 (3H, s), 2.99 (6H, s). | 379.0; 98.0 |
| Product 21 | 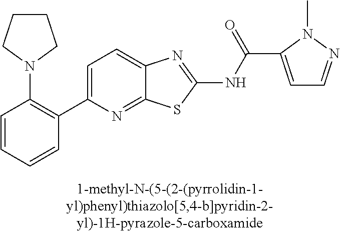 [SMILES: Cn1nccc1C(=O)Nc1nc2ccc(-c3ccccc3N3CCCC3)nc2s1] | (DMSO-d₆, 400 MHz): δH 13.00 (1H, s), 8.12 (1H, d, J = 8.4 Hz), 7.59-7.66 (2H, m), 7.44 (1H, s), 7.34 (1H, dd, J = 1.6 Hz, J = 7.6 Hz), 7.23-7.29 (1H, m), 6.91 (1H, d, J = 8.4 Hz), 6.84 (1H, t, J = 7.2 Hz), 4.17 (3H, s), 2.87 (4H, brs), 1.73 (4H, brs). | 405.1; 96.0 |
| Product 22 | 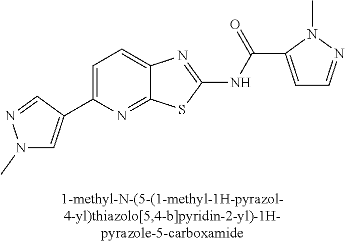 [SMILES: Cn1cc(-c2ccc3nc(NC(=O)c4ccnn4C)sc3n2)cn1] | (400 MHz, DMSO-d₆) δH 12.95 (1H, s), 8.38 (1H, s), 8.10 (1H, d, J = 8.4 Hz), 8.06 (1H, s), 7.78 (1H, d, J = 8.4 Hz), 7.60 (1H, d, J = 2.4 Hz), 7.42 (1H, s), 4.17 (3H, s), 3.90 (3H, s). | 340.2, 99.0 |
| Product 23 | 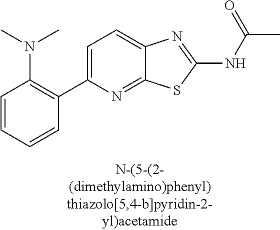 [SMILES: CC(=O)Nc1nc2ccc(-c3ccccc3N(C)C)nc2s1] | (400 MHz, DMSO-d₆) δH 12.46 (1H, s), 8.09 (1H, d, J = 8.8 Hz), 7.97 (1H, d, J = 8.4 Hz), 7.51 (1H, dd, J = 1.2 Hz, 7.6 Hz), 7.29-7.35 (1H, m), 7.11 (1H, d, J = 8.0 Hz), 7.04 (1H, t, J = 7.6 Hz), 2.53 (6H, s), 2.23 (3H, s). | 313.2; 99.0 |
| Product 24^{b} | 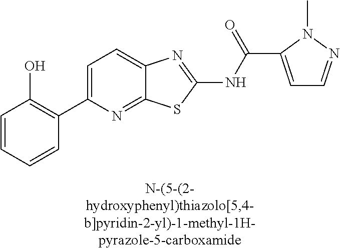 [SMILES: Cn1nccc1C(=O)Nc1nc2ccc(-c3ccccc3O)nc2s1] | (400 MHz, DMSO-d₆) δH 4.18 (3H, s), 6.92-7.01 (2H, m), 7.24-7.35 (2H, m), 7.57 (1H, s), 7.99 (1H, d, J = 7.6 Hz), 8.15-8.26 (2H, m), 12.29 (1H, s), 13.09 (1H, s). | 352.1; 99.0 |
| Product 25^{c} | 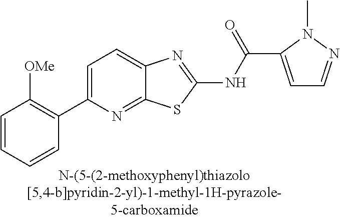 [SMILES: COc1ccccc1-c1ccc2nc(NC(=O)c3ccnn3C)sc2n1] | (DMSO-d₆, 400 MHz): δH 13.00 (1H, s), 8.12 (1H, d, J = 8.0 Hz), 7.95 (1H, d, J = 8.4 Hz), 7.78 (1H, dd, J = 1.6 Hz, J = 7.6 Hz), 7.59 (1H, d, J = 2.0 Hz), 7.38-7.45 (2H, m), 7.18 (1H, d, J = 8.0 Hz), 7.10 (1H, t, J = 8.0 Hz), 4.18 (3H, s), 3.86 (s, 3H). | 366.1; 94.0 |
| Product 26^{c} | 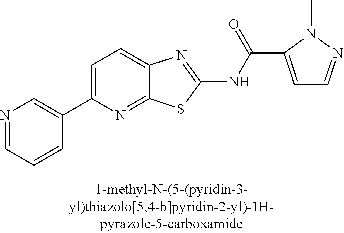 [SMILES: Cn1nccc1C(=O)Nc1nc2ccc(-c3cccnc3)nc2s1] | (DMSO-d₆, 400 MHz): δH 13.09 (1H, s), 9.33 (1H, d, J = 1.6 Hz), 8.65 (1H, d, J = 4 Hz), 8.51 (1H, d, J = 8.0 Hz), 8.23 (2H, q, J = 8.4 Hz, J = 24.4 Hz), 7.61 (1H, d, J = 2 Hz), 7.55 (1H, q, J = 4.8 Hz, J = 8 Hz), 7.44 (1H, d, J = 1.2 Hz), 4.18 (3H, s). | 337.1; 95.0 |

^{a}The reaction was performed in a microwave
^{b}The reaction was performed using Na₂CO₃ as base, dioxane/water (3/1) as solvent, Pd(dppf)Cl₂ as catalyst and conducted in a microwave
^{c}The reaction was performed using K₂CO₃ as base, dioxane/water (3/1) as solvent, cyclopentyl(diphenyl)phosphane-dichloromethane-dichloropalladium-iron as catalyst and conducted in a microwave.

### Example 6: Preparation of 1-methyl-N-(5-(pyridin-2-yl)thiazolo[5,4-b]pyridin-2-yl)-1H-pyrazole-5-carboxamide (Product 27)

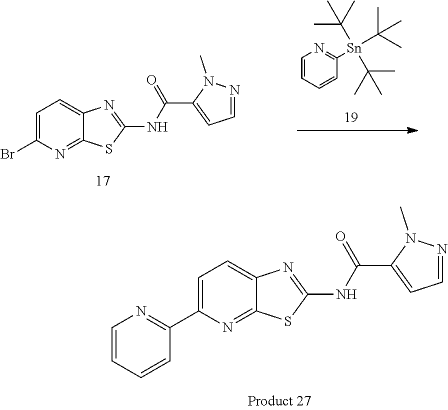 [SMILES: Cn1nccc1C(=O)Nc1nc2ccc(Br)nc2s1 ; C[C](C)(C)[Sn]([c]1ccccn1)([C](C)(C)C)[C](C)(C)C ; Cn1nccc1C(=O)Nc1nc2ccc(-c3ccccn3)nc2s1]

Tetrakis(triphenylphosphine)palladium(0) (Pd(PPh₃)₄, 205.02 mg, 177.42 mmol) was added to a solution of N-(5-bromothiazolo[5,4-b]pyridin-2-yl)-1-methyl-1H-pyrazole-5-carboxamide (17, 200 mg, 591.40 mmol) and 2-(tri-tert-butylstannyl)pyridine (19, 435.44 mg, 1.18 mmol) in toluene (3.0 mL) and dioxane (3.0 mL). The reaction mixture was stirred at 100° C. for 2 hours in a microwave. The mixture was filtered and the filtrate was concentrated. The residue was purified by silica gel column chromatography. The product was further purified by prep-HPLC to provide the free base of the title compound (Product 27, 45 mg, 131.11 mmol, 20% yield). ¹H-NMR (DMSO-d₆, 400 MHz): δH 13.11 (1H, s), 8.71 (1H, d, J=3.6 Hz), 8.55 (1H, d, J=8.4 Hz), 8.45 (1H, d, J=8.0 Hz), 8.26 (1H, d, J=8.4 Hz), 7.97 (1H, t, J=8.4 Hz), 7.61 (1H, s), 7.43-7.47 (2H, m), 4.18 (3H, s); MS (m/z):337.1 [M+H]⁺; purity 98%.

### Example 7: Preparation of 1-methyl-N-(5-(thiazol-2-yl)thiazolo[5,4-b]pyridin-2-yl)-1H -pyrazole-5-carboxamide Product 28

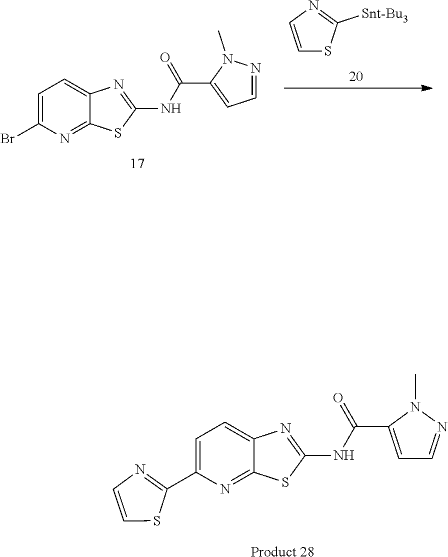

Cesium fluoride (134.75 mg, 887.09 mmol, 32.71 mL), CuI (168.95 mg, 887.09 mmol) and Pd(PPh₃)₄ (136.68 mg, 118.28 mmol) were added to a solution of N-(5-bromothiazolo[5,4-b]pyridin-2-yl)-1-methyl-1H-pyrazole-5-carboxamide (17) (200 mg, 591.40 mmol) and 2-(tri-tert-butylstannyl)thiazole (20, 331.93 mg, 887.09 mmol) in dioxane (5.0 mL). The mixture was stirred at 120° C. for 3 hours. The mixture was filtered and the filtrate was evaporated under reduced pressure. The crude product was purified by re-crystallization from PE:EtOAc=20:1 (5.0 mL) at 25° C. to provide the free base of the title compound (Product 28, 18 mg, 8.9% yield). ¹H-NMR (DMSO-d₆, 400 MHz): δH 13.16 (1H, s), 8.29 (2H, s), 8.01 (1H, d, J=3.2 Hz), 7.90 (1H, d, J=3.2 Hz), 7.62 (1H, d, J=2.0 Hz), 7.45 (1H, s), 4.18 (3H, s); MS (m/z):343.1 [M+H]⁺; purity 95%.

### Example 8: Preparation of N-(5-(4,5-dimethylthiazol-2-yl)thiazolo[5,4-b]pyridin-2-yl)-1-methyl-1H-pyrazole-5-carboxamide (Product 29)

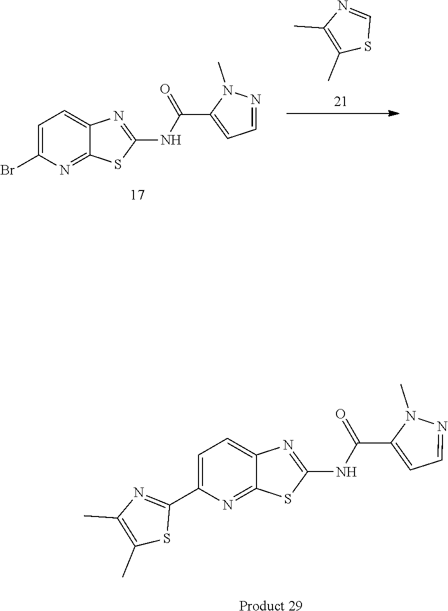

4,5-Dimethylthiazole (21, 401.61 mg, 3.55 mmol), Pd(OAc)₂ (26.55 mg, 118.28 mmol) and tributylphosphine (25.19 mg, 118.28 mmol, 30.72 mL, 95% purity) were added to a solution of N-(5-bromothiazolo[5,4-b]pyridin-2-yl)-1-methyl-1H-pyrazole-5-carboxamide (17, 400 mg, 1.18 mmol), Cs₂CO₃ (423.91 mg, 1.30 mmol) and CuBr (33.93 mg, 236.56 mmol, 7.20 mL) in DMF (10 mL). The mixture was stirred at 150° C. for 12 hours in a microwave. The mixture was filtered and the filtrate was concentrated. The residue was purified by silica gel column chromatography and then was further purified by prep-HPLC to get the title compound (Product 29, 44 mg, 11% yield). 1H-NMR (400 MHz, DMSO-d₆): δ ppm 2.35 (s, 3H), 2.42 (s, 3H), 4.17 (s, 3H), 7.44 (s, 1H), 7.61 (d, J=2.0 Hz, 1H), 8.17-8.23 (m, 2H), 13.14 (s, 1H), MS: [M+H]⁺, 371.1; purity 98%.

Final compounds reported in Table 5 were prepared according to the procedure reported for Product 29 using the corresponding boronic acid:

TABLE 5

| | | | MS (m/z) |
|---|---|---|---|
| | | | [M + H]⁺; |
| Final compound | Structure and name | ¹H-NMR | Purity (%) |
| Product 30 | 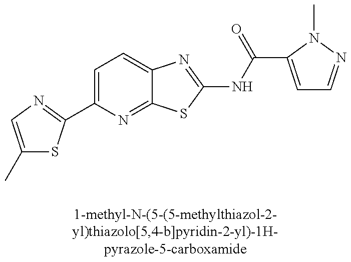 [SMILES: Cc1cnc(-c2ccc3nc(NC(=O)c4ccnn4C)sc3n2)s1] | (400 MHz, DMSO-d₆) δH 2.52 (s, 3H), 4.18 (s, 3H), 7.42 (s, 1H), 7.60 (d, J = 2.0 Hz, 1H), 7.68 (d, J = 1.2 Hz, 1H), 8.22 (q, J = 8.8 Hz, J = 11.6 Hz, 2H), 13.1 (s, 1H). | 357.0; 99.0 |
| Product 31* | 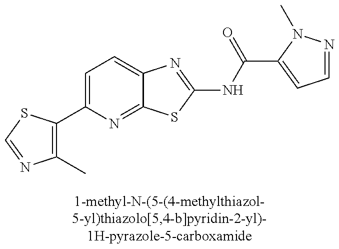 [SMILES: Cc1ncsc1-c1ccc2nc(NC(=O)c3ccnn3C)sc2n1] | (400 MHz, DMSO-d₆) δ ppm 2.70 (s, 3H), 4.17 (s, 3H), 7.37 (d, J = 2.0 Hz, 1H), 7.58 (d, J = 1.6 Hz, 1H), 7.81 (J = 8.4 Hz, 1H), 8.17 (d, J = 8.4 Hz, 1H), 9.04 (s, 1H). | 357.1; 97.0 |
| Product 32* | 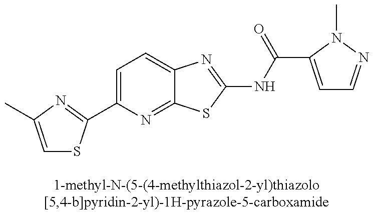 [SMILES: Cc1csc(-c2ccc3nc(NC(=O)c4ccnn4C)sc3n2)n1] | (400 MHz, DMSO-d₆) δ ppm 2.48 (s, 3H), 4.17 (s, 3H), 7.44 (s, 2H), 7.61 (s, 1H), 8.24 (s, 2H), 13.14 (s, 1H) | 357.1; 94.0 |
| Product 33 | 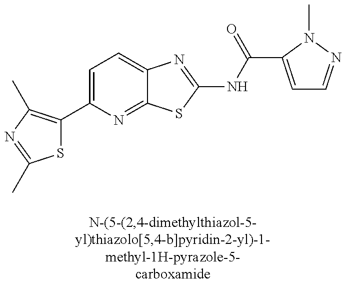 [SMILES: Cc1nc(C)c(-c2ccc3nc(NC(=O)c4ccnn4C)sc3n2)s1] | (400 MHz, DMSO-d₆) δ ppm 2.62 (s, 3H), 2.63 (s, 3H), 4.16 (s, 3H), 7.42 (s, 1H), 7.60 (d, J = 2.0 Hz, 1H), 7.76 (d, J = 8.4 Hz, 1H), 8.18 (d, J = 8.4 Hz, 1H), 13.07 (s, 1H). | 371.0; 91.0 |

*Product 31 and Product 32 were obtained by reactng N-(5-bromothiazolo[5,4-b]pyridin-2-yl)-1-methyl-1H-pyrazole-5-carboxamide and 4-methylthiazole and separated by reverse phase prep-HPLC.

### Example 9: Preparation of N-(5-(2-isobutyramidophenyl)thiazolo[5,4-b]pyridin-2-yl)-1-methyl-1H-pyrazole-5-carboxamide (Product 34)

### Step 1. Preparation of N-(5-(2-aminophenyl)thiazolo[5,4-b]pyridin-2-yl)-1-methyl pyrazole-5-carboxamide (23)

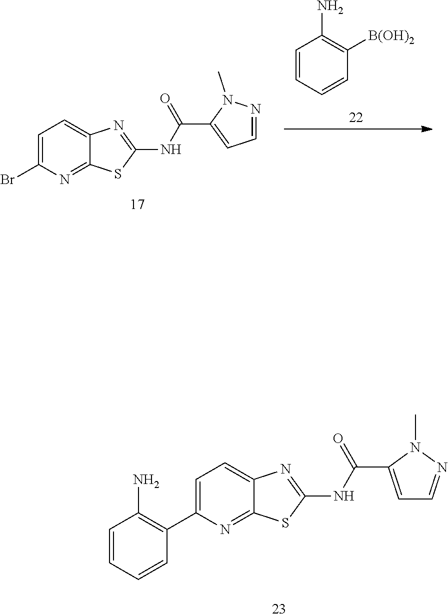 [SMILES: Cn1nccc1C(=O)Nc1nc2ccc(Br)nc2s1 ; Nc1ccccc1B(O)O ; Cn1nccc1C(=O)Nc1nc2ccc(-c3ccccc3N)nc2s1 ; "23"]

Compound 23 was synthesized according to step 3 for the synthesis of Product 1, starting from N-(5-bromothiazolo[5,4-b]pyridin-2-yl)-1-methyl-1H-pyrazole-5-carboxamide (17) and (2-aminophenyl)boronic acid (22).

### Step 2. Preparation of N-(5-(2-isobutyramidophenyl)thiazolo[5,4-b]pyridin-2-yl)-1-methyl-1H-pyrazole-5-carboxamide (Product 34)

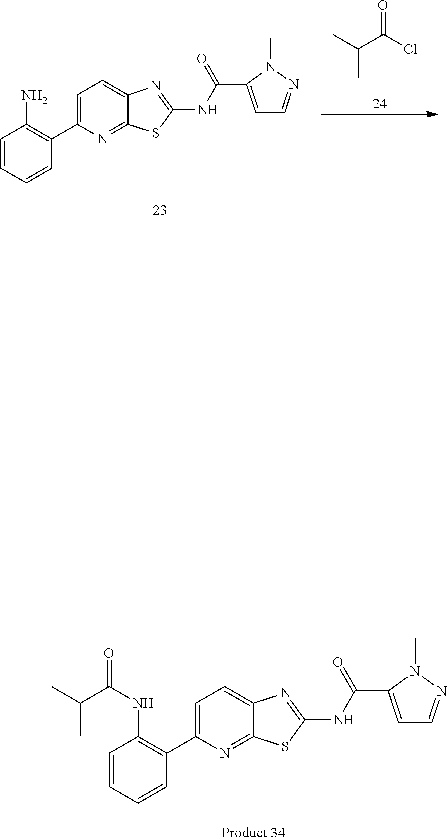

Triethylamine (173.27 mg, 1.71 mmol, 238.34 mL) and DMAP (69.73 mg, 570.78 mmol) were added to a solution of N-(5-(2-aminophenyl)thiazolo[5,4-b]pyridin-2-yl)-1-methyl-1H-pyrazole-5-carboxamide (23, 200 mg, 570.78 mmol) in DCM (5 mL). Isobutyryl chloride (24, 63.86 mg, 599.32 mmol, 62.61 mL) was added to the reaction mixture at 0° C. The mixture was stirred at 15° C. for 2 hours. The solvent was removed under reduced pressure. The crude product was purified by re-crystallization from MeOH (5.0 mL) at 15° C. to provide the free base of the title compound (Product 34, 123 mg, 51% yield). ¹H-NMR (DMSO-d₆, 400 MHz): δH 13.13 (1H, s), 10.98 (1H, s), 8.27 (1H, d, J=8.4 Hz), 8.13 (1H, d, J=8.0 Hz), 7.91 (1H, d, J=8.4 Hz), 7.79 (1H, d, J=6.8 Hz), 7.62 (1H, d, J=2.4 Hz), 7.42-7.45 (2H, m), 7.26 (1H, t, J=7.6 Hz), 4.18 (3H, s), 2.50-2.59 (1H, m), 1.14 (6H, d, J=7.2 Hz); MS (m/z):421.2 [M+H]⁺; purity 97%.

### Example 10: Preparation of N-(5-(2-acetamidophenyl)thiazolo[5,4-b]pyridin-2-yl)-1-methyl-1H-pyrazole-5-carboxamide (Product 35)

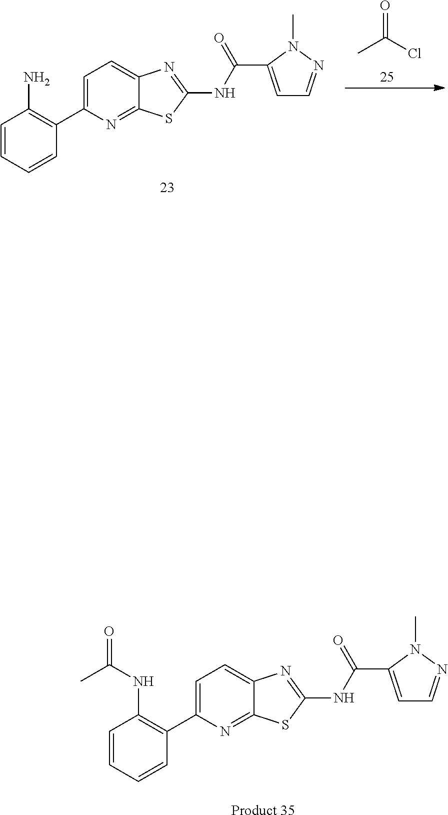

Product 35 was synthesized according to the procedure reported for Product 34, starting from N-(5-(2-aminophenyl)thiazolo[5,4-b]pyridin-2-yl)-1-methyl-1H-pyrazole-5-carboxamide (23) and acetyl chloride (25). ¹H-NMR (DMSO-d₆, 400 MHz): δH 13.10 (1H, s), 10.61 (1H, s), 8.25 (1H, d, J=8.8 Hz), 7.97 (1H, d, J=7.6 Hz), 7.84 (1H, d, J=8.8 Hz), 7.73-7.77 (1H, m), 7.61 (1H, d, J=2.0 Hz), 7.40-7.47 (2H, m), 7.27 (1H, t, J=7.6 Hz), 4.18 (3H, s), 2.03 (3H, s); MS (m/z):393.3 [M+H]⁺; purity 98%.

### Example 11: Preparation of 1-methyl-N-(5-(2-(N-methylacetamido)phenyl)thiazolo[5,4-b]pyridin-2-yl)-1H-pyrazole-5-carboxamide (Product 36)

### Step 1. Preparation of 1-methyl-N-(5-(2-(methylamino)phenyl)thiazolo[5,4-b]pyridin-2-yl)-1H-pyrazole-5-carboxamide (26)

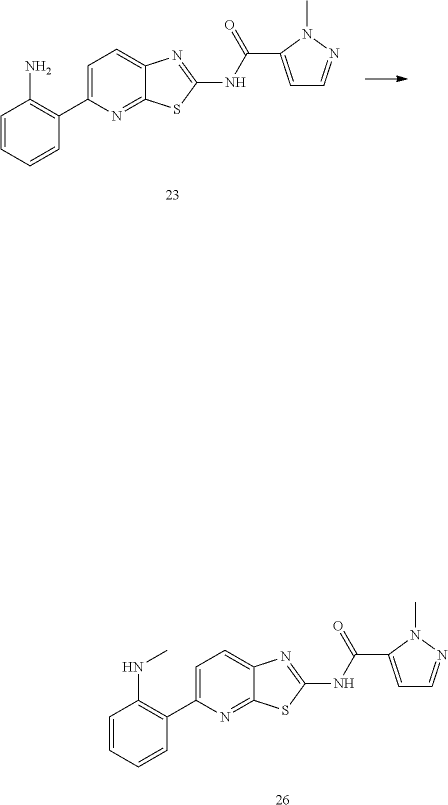 [SMILES: Cn1nccc1C(=O)Nc1nc2ccc(-c3ccccc3N)nc2s1 ; "23" ; CNc1ccccc1-c1ccc2nc(NC(=O)c3ccnn3C)sc2n1 ; "26"]

Paraformaldehyde (321.06 mg, 10.70 mmol) was added to a solution of N-(5-(2-aminophenyl)thiazolo[5,4-b]pyridin-2-yl)-1-methyl-1H-pyrazole-5-carboxamide (23, 1.5 g, 4.28 mmol) in DMF (30 mL) at 15° C. The mixture was stirred at 15° C. for 0.5 hour. Subsequently, NaBH₃CN (1.61 g, 25.69 mmol) was added at 15° C. The mixture was stirred at 50° C. or 15.5 hours. The reaction mixture was diluted with water (30 mL) and extracted three times with EtOAc (30 mL). The combined organic layers were washed with water (50 mL), dried over anhydrous sodium sulfate, filtered and concentrated under reduced pressure to give a residue. The residue was purified by silica gel column chromatography to provide the free base of the title compound (26, 850 mg, 27% yield).

### Step 2. Preparation of 1-methyl-N-(5-(2-(N-methylacetamido)phenyl)thiazolo[5,4-b]pyridin-2-yl)-1H-pyrazole-5-carboxamide (Product 36)

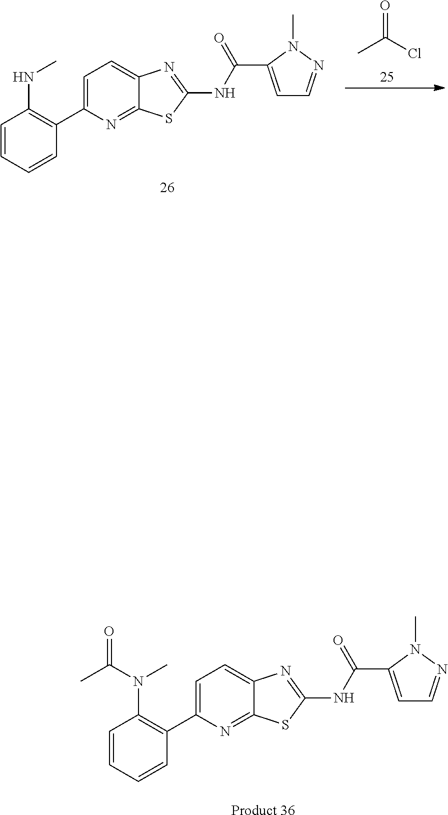

Product 36 was prepared according to the procedure reported for step 2 for the synthesis of Product 34 starting from 1-methyl-N-(5-(2-(methylamino)phenyl)thiazolo[5,4-b]pyridin-2-yl)-1H-pyrazole-5-carboxamide (26) and acetyl chloride (25). ¹H-NMR (DMSO-d₆, 400 MHz): δH 13.08 (1H, s), 8.23 (1H, d, J=8.4 Hz), 7.75 (1H, dd, J=1.6 Hz, J=6.8 Hz), 7.59-7.64 (2H, m), 7.51-7.58 (2H, m), 7.45-7.50 (1H, m), 7.43 (1H, brs), 4.17 (3H, s), 2.95 (3H, s), 1.64 (3H, s); MS (m/z):407.1 [M+H]⁺; purity 99%.

### Example 12: Preparation of N-(5-(2-((2-hydroxyethyl)(methyl)amino)phenyl)thiazolo[5,4-b]pyridin-2-yl)-1-methyl-1H-pyrazole-5-carboxamide (Product 37)

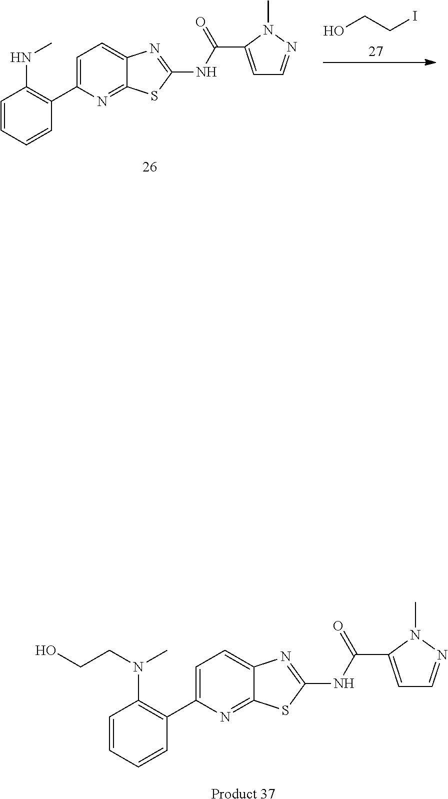

A solution of 1-methyl-N-(5-(2-(methylamino)phenyl)thiazolo[5,4-b]pyridin-2-yl)-1H-pyrazole-5-carboxamide (26, 400 mg, 1.10 mmol) and 2-iodoethan-1-ol (27, 1.89 g, 10.98 mmol, 857.97 mL) was stirred at 50° C. for 16 hours. The reaction mixture was concentrated under reduced pressure to give a residue. The residue was purified by prep-HPLC to provide the free base of the title compound (Product 37, 203 mg, 44% yield). ¹H-NMR (DMSO-d₆, 400 MHz): δH 13.00 (1H, s), 8.14 (1H, d, J=8.4 Hz), 8.05 (1H, d, J=8.4 Hz), 7.61 (1H, d, J=1.6 Hz), 7.53 (1H, d, J=7.2 Hz), 7.45 (1H, s), 7.34 (1H, t, J=7.2 Hz), 7.18 (1H, d, J=8.0 Hz), 7.07 (1H, t, J=7.2 Hz), 4.49 (1H, t, J=5.2 Hz), 4.18 (3H, s), 3.40 (2H, q, J=6.0 Hz, J=11.6 Hz), 2.92 (2H, t, J=6.0 Hz), 2.59 (3H, s); MS (m/z):409.2 [M+H]⁺; purity 97%.

### Example 13: Preparation of N-methyl-N-(5-(o-tolyl)thiazolo[5,4-b]pyridin-2-yl)picolinamide (Product 38)

### Step 1. Preparation of 5-bromo-2-chlorothiazolo[5,4-b]pyridine (28)

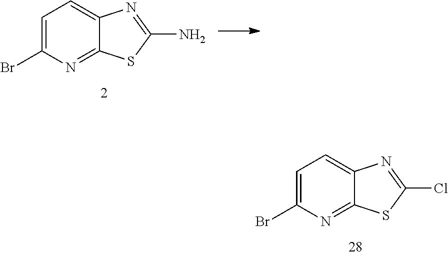 [SMILES: Nc1nc2ccc(Br)nc2s1 ; Clc1nc2ccc(Br)nc2s1]

Copper (II) chloride (8.65 g, 64.32 mmol) was added at 0° C. to a mixture of 5-bromothiazolo[5,4-b]pyridin-2-amine (2, 10 g, 43.46 mmol) in ACN (200 mL). The reaction mixture was stirred at 0° C. for 0.5 h. Then a solution of tert-butyl nitrite (6.59 g, 63.90 mmol) in ACN (25 mL) was added drop-wise at 0° C. to the reaction mixture. The reaction mixture was stirred at 25° C. for 11.5 h. The residue was diluted with water (100 mL) and the aqueous phase was extracted three times with ethyl acetate (50 mL). The combined organic phase was washed with brine (50 mL), dried with anhydrous Na₂SO₄, filtered and concentrated in vacuum to provide the free base of the title compound (28, 8.0 g, 67% yield). ¹H-NMR (CDCl₃-d₁, 400 MHz): δH 8.04 (1H, d, J=8.4 Hz), 7.62 (1H, d, J=8.4 Hz).

### Step 2. Preparation of 5-bromo-N-methylthiazolo[5,4-b]pyridin-2-amine (29)

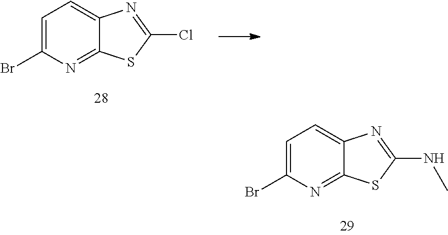

A solution of 5-bromo-2-chlorothiazolo[5,4-b]pyridine (28, 2 g, 8.02 mmol) in MeNH2/EtOH (829.79 mg, 8.02 mmol, 30 mL, 30% purity) was stirred at 60° C. for 12 hours in a sealed tube. The mixture was concentrated under reduced pressure. The residue was purified by silica gel column chromatography to provide the free base of the title compound (29, 0.4 g, 20% yield). ¹H-NMR (CDCl₃-d₁, 400 MHz): δH 7.57 (1H, d, J=8.4 Hz), 7.37 (1H, d, J=8.4 Hz), 5.40 (1H, s), 3.14 (3H, s).

### Step 3. Preparation of N-methyl-5-(o-tolyl)thiazolo[5,4-b]pyridin-2-amine (31)

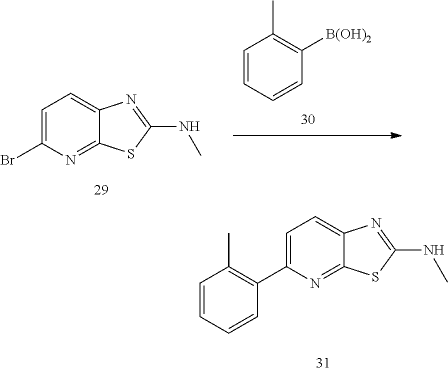

Compound 31 was synthesized according to step 3 for the synthesis of Product 1 using K₂CO₃ as base, dioxane/water as solvent, Pd(dppf)Cl₂·CH₂Cl₂ as catalyst. ¹H-NMR (DMSO-d₆, 400 MHz): δH 8.27 (1H, d, J=4.4 Hz), 7.73 (1H, d, J=8.4 Hz), 7.35-7.42 (2H, m), 7.23-7.31 (3H, m), 2.98 (3H, s), 2.33 (3H, s).

### Step 4. Preparation of N-methyl-N-(5-(o-tolyl)thiazolo[5,4-b]pyridin-2-yl)picolinamide (Product 38)

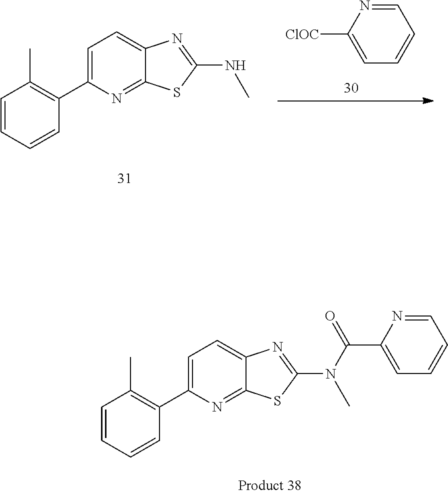

Product 38 was synthesized according to step 6 for the synthesis of Product 1 modification D, starting from N-methyl-5-(o-tolyl)thiazolo[5,4-b]pyridin-2-amine (31) and picolinoyl chloride (32). ¹H-NMR (DMSO-d₆, 400 MHz): δH 8.74 (1H, d, J=4.0 Hz), 8.31 (1H, d, J=8.4 Hz), 8.09 (1H, td, J=2.0 Hz, J=7.6 Hz), 7.91 (1H, d, J=8.0 Hz), 7.63-7.71 (2H, m), 7.48 (1H, d, J=6.8 Hz), 7.28-7.37 (3H, m), 3.71 (3H, s), 2.38 (3H, s); MS (m/z):361.1 [M+H]⁺; purity 99%.

### Example 14: Preparation of N-(5-(o-tolyl)thiazolo[5,4-b]pyridin-2-yl)morpholine-4-carboxamide (Product 39)

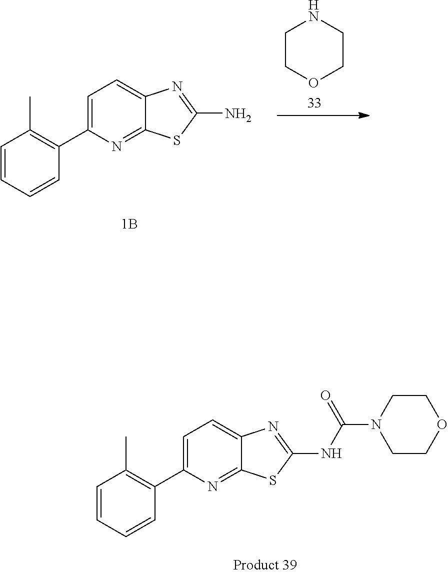 [SMILES: Cc1ccccc1-c1ccc2nc(N)sc2n1 ; C1COCCN1 ; Cc1ccccc1-c1ccc2nc(NC(=O)N3CCOCC3)sc2n1]

A solution of 5-(o-Tolyl)thiazolo[5,4-b]pyridin-2-amine (1B, 200 mg, 828.81 mmol) and TEA (125.80 mg, 1.24 mmol, 173.04 uL) in THF (5.0 mL) was added at 0° C. to a solution of triphosgene (86.08 mg, 290.08 mmol) in THF (4.0 mL). Subsequently, morpholine (33, 288.82 mg, 3.32 mmol, 291.74 mL) was added into the mixture at 0° C. and the reaction mixture was stirred at 25° C. for 2 hours. Water (1.0 mL) was added and the mixture was extracted three times with EtOAc (1.0 mL). The combined organic layers were dried over anhydrous sodium sulfate, filtered and concentrated. The residue was purified by prep-HPLC to provide the free base of the title compound (Product 39, 83 mg, 28% yield). ¹H-NMR (DMSO-d₆, 400 MHz): δH 11.43 (1H, s), 8.03 (1H, brs), 7.53 (1H, d, J=8.8 Hz), 7.42 (1H, d, J=6.8 Hz), 7.25-7.35 (3H, m), 3.60-3.65 (4H, m), 3.51-3.59 (4H, m), 2.35 (3H, s); MS (m/z):355.1 [M+H]⁺; purity 98%.

### Example 15: Preparation of 4-methyl-N-(5-(o-tolyl)thiazolo[5,4-b]pyridin-2-yl)piperazine-1-carboxamide (Product 40)

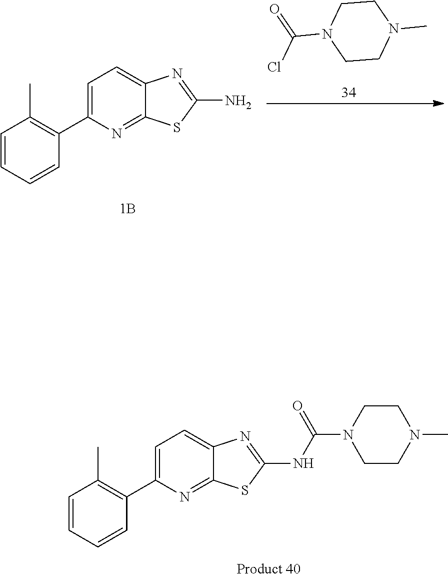

Sodium hydride (397.79 mg, 9.95 mmol, 60% purity) was added at 0° C. to a solution of 5-(o-Tolyl)thiazolo[5,4-b]pyridin-2-amine (1B, 400 mg, 1.66 mmol) in THF (10 mL). The reaction mixture was stirred for 30 min and then 4-methylpiperazine-1-carbonyl chloride (35, 1.32 g, 6.63 mmol, 1.10 mL, HCl) was added at 0° C. Subsequently, the reaction mixture was stirred at 25° C. for 7.5 hours. After the addition of water (20 mL), the mixture was extracted three times with EtOAc (20 mL). The combined organic layers were washed three times with brine (10 mL), dried over anhydrous sodium sulfate, filtered and concentrated. The residue was purified by prep-HPLC to provide the free base of the title compound (Product 40, 126 mg, 20% yield). ¹H NMR (DMSO-d₆, 400 MHz): δH 11.60 (1H, s), 7.96 (1H, d, J=8.4 Hz), 7.53 (1H, d, J=8.4 Hz), 7.43 (1H, d, J=6.8 Hz), 7.26-7.32 (3H, m), 3.57 (4H, brs), 2.31-2.38 (7H, m), 2.21 (3H, s); MS (m/z):386.1 [M+H]⁺; purity 95%.

### Example 16: Preparation of 1-methyl-N-(5-(o-tolyl)thiazolo[4,5-b]pyridin-2-yl)-1H-pyrazole-5-carboxamide (Product 41)

### Step 1. Preparation of 5-chlorothiazolo[4,5-b]pyridine-2-thiol (37)

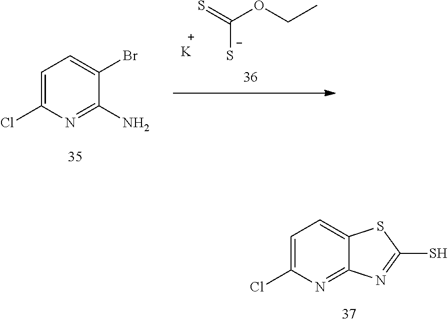

A solution of 3-bromo-6-chloropyridin-2-amine (35, 4 g, 19.28 mmol) and ethoxycarbothioylsulfanyl potassium (36, 5.56 g, 34.71 mmol) in DMF (25 mL) was stirred at 150° C. for 4 hours. Water (30 mL) was added and then the mixture was extracted three times with EtOAc (20 mL). The water phase was adjusted to pH=3 with conc. HCl. A yellow solid was precipitated, filtered and dried to provide the title compound 5-chlorothiazolo[4,5-b]pyridine-2-thiol (37, 1.5 g, 34% yield). ¹H-NMR (400 MHz, DMSO-d6): δ_{H} 7.41 (d, J=8.4 Hz, 1H), 8.15 (d, J=8.4 Hz, 1H), 14.5 (s, 1H).

### Step 2. Preparation of 5-chloro-2-(methylthio)thiazolo[4,5-b]pyridine (38)

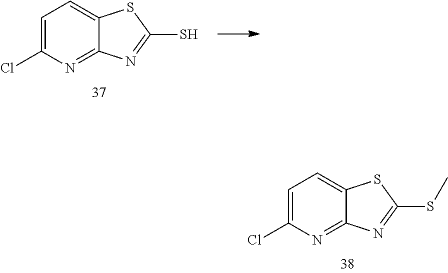

Potassium carbonate (2.05 g, 14.80 mmol) was added to a solution of 5-chlorothiazolo[4,5-b]pyridine-2-thiol (37, 1.5 g, 7.40 mmol) in THF (30 mL). The reaction mixture was cooled to 0° C. and then MeI (1.58 g, 11.10 mmol, 691.08 mL) was added. The mixture was stirred at 25° C. for 2 hours. Water (10 mL) was added at 0° C. and the mixture was extracted three times with EtOAc (10 mL). The combined organic layers were dried over anhydrous sodium sulfate, filtered and concentrated to provide the title compound 5-chloro-2-(methylthio)thiazolo[4,5-b]pyridine (38, 1.2 g, crude). ¹H-NMR (400 MHz, DMSO-d6): δH 2.83 (s, 3H), 7.48 (d, J=8.4, 1H), 8.54 (d, J=8.4 Hz, 1H).

### Step 3. Preparation of 5-chlorothiazolo[4,5-b]pyridin-2-amine (39)

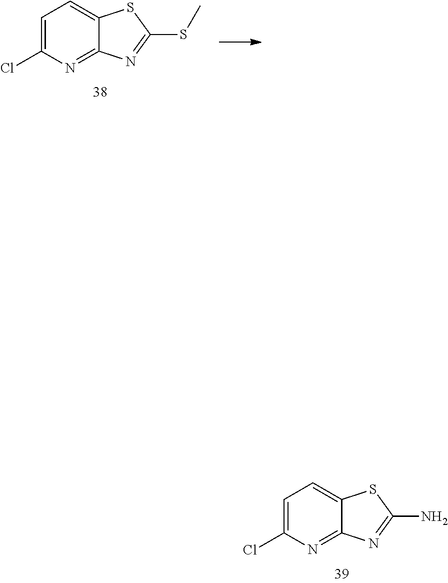

5-Chloro-2-(methylthio)thiazolo[4,5-b]pyridine (38, 700 mg, 3.23 mmol, 1 eq) was dissolved with NH₃. Water (20 mL) and EtOH (20 mL) in a 100 mL sealed tube. The solution was stirred at 100° C. for 12 hours. The mixture was cooled to 25° C. The mixture was filtered and the filter cake was dried to afford the title compound 5-chlorothiazolo[4,5-b]pyridin-2-amine (39, 340 mg, 51% yield). ¹H-NMR (400 MHz, DMSO-d6): δH 7.05 (d, J=8.0, 1H), 8.08 (d, J=8.4 Hz, 1H), 8.18 (s, 2H).

### Step 4. Preparation of N-(5-chlorothiazolo[4,5-b]pyridin-2-yl)-1-methyl-1H-pyrazole-5-carboxamide (40)

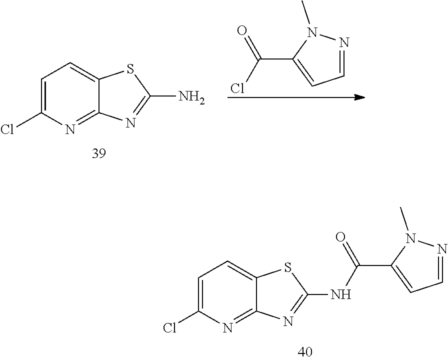

4-Dimethylaminopyridine (671.29 mg, 5.49 mmol), TEA (556.01 mg, 5.49 mmol, 764.80 mL) and 1-methyl-1H-pyrazole-5-carbonyl chloride (794.31 mg, 5.49 mmol) were added in sequence to a solution of 5-chlorothiazolo[4,5-b]pyridin-2-amine (39, 340 mg, 1.83 mmol) in THF (20 mL) at 0° C. The solution was stirred at 25° C. for 5 hours. Water was added (20 mL) to the mixture and a precipitate formed. The mixture was filtered, and the precipitate was dried to afford the title compound N-(5-chlorothiazolo[4,5-b]pyridin-2-yl)-1-methyl-1H-pyrazole-5-carboxamide (40, 400 mg, 1.23 mmol, 67% yield). ¹H-NMR (400 MHz, DMSO-d6): δ_{H} 4.17 (s, 3H), 7.44 (d, J=8.0 Hz, 2H), 7.61 (d, J=7.2 Hz, 1H), 8.54 (d, J=8.0 Hz, 1H), 13.3 (s, 1H).

### Step 5. Preparation of 1-methyl-N-(5-(o-tolyl)thiazolo[4,5-b]pyridin-2-yl)-1H-pyrazole-5-carboxamide (Product 41)

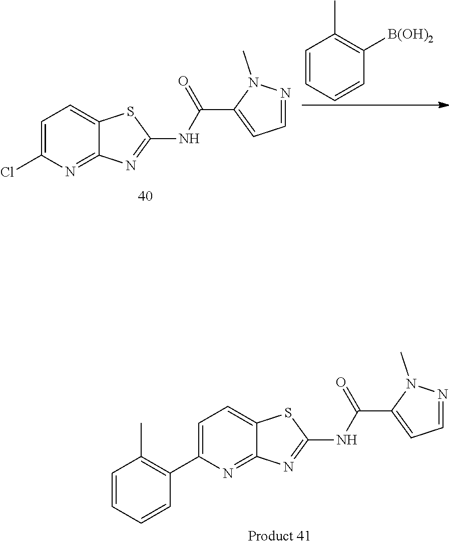

Potassium carbonate (70.58 mg, 510.67 mmol) and Pd(dppf)Cl₂·CH₂C₁₂ (27.80 mg, 34.04 mmol), were added to a solution of N-(5-chlorothiazolo[4,5-b]pyridin-2-yl)-1-methyl-1H-pyrazole-5-carboxamide (40, 50 mg, 170.22 mmol) and o-tolylboronic acid (46.29 mg, 340.45 mmol) in dioxane (3.0 mL) and water (1.0 mL). The reaction mixture was stirred at 100° C. for 45 min in a microwave. The mixture was filtered and concentrated. The residue was purified by silica gel column chromatography to afford the title compound 1-methyl-N-(5-(o-tolyl)thiazolo[4,5-b]pyridin-2-yl)-1H-pyrazole-5-carboxamide (Product 41, 67 mg, 27% yield). ¹H-NMR (400 MHz, DMSO-d6): δ_{H} 2.38 (s, 3H), 4.18 (s, 3H), 7.29-7.37 (m, 3H), 7.45-7.50 (m, 3H), 7.61 (d, J=1.6 Hz, 1H), 8.55 (d, J=8.0 Hz, 1H), 13.2 (s, 1H); MS: 350.1 [M+H]⁺; purity 97%.

### Example 17: Preparation of 1-methyl-N-(6-(o-tolyl)thiazolo[4,5-c]pyridin-2-yl)-1H-pyrazole-5-carboxamide (Product 42)

### Step 1. Preparation of 6-chlorothiazolo[4,5-c]pyridin-2-amine (42)

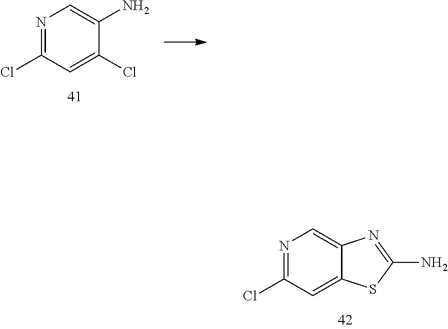 [SMILES: Nc1cnc(Cl)cc1Cl ; Nc1nc2cnc(Cl)cc2s1]

Concentrated HCl (12 M, 332.50 mL) was added to a solution of 4,6-dichloropyridin-3-amine (41, 5.0 g, 30.67 mmol) and KSCN (8.94 g, 92.02 mmol, 8.94 mL) in dioxane (100 mL). The reaction mixture was stirred at 110° C. for 12 hours. The mixture was filtered and the filtrate was concentrated. The residue was purified by silica gel column chromatography to provide the free base of the title compound 6-chlorothiazolo[4,5-c]pyridin-2-amine (42, 163 mg, 110% yield). MS (m/z):185.9 [M+H]⁺.

### Step 2. Preparation of tert-butyl (6-chlorothiazolo[4,5-c]pyridin-2-yl)carbamate (43)

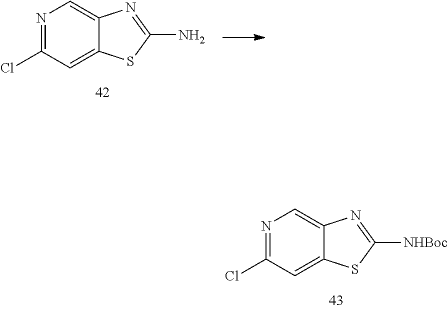 [SMILES: Nc1nc2cnc(Cl)cc2s1 ; CC(C)(C)OC(=O)Nc1nc2cnc(Cl)cc2s1]

Di-tert-butyl dicarbonate (8.82 g, 40.40 mmol, 9.28 mL) was added to a solution of 6-chlorothiazolo[4,5-c]pyridin-2-amine (42, 2.5 g, 13.47 mmol) in DCM (40 mL), followed by DMAP (329.06 mg, 2.69 mmol) and TEA (1.50 g, 14.81 mmol, 2.06 mL). The reaction mixture was stirred at 25° C. for 12 hours. The mixture was concentrated. The residue was purified by silica gel column chromatography to provide the free base of the title compound tert-butyl (6-chlorothiazolo[4,5-c]pyridin-2-yl)carbamate (43, 1 g, 3.15 mmol, 23% yield). MS (m/z): 286.0 [M+H]⁺.

### Example 18: Preparation of tert-butyl (6-(o-tolyl)thiazolo[4,5-c]pyridin-2-yl)carbamate (44)

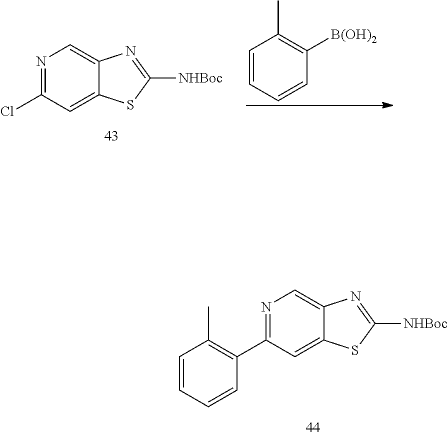

Cesium carbonate (1.14 g, 3.50 mmol) and Pd(PPh₃)₂C₁₂ (245.63 mg, 349.96 mmol) were added to a solution of tert-butyl (6-chlorothiazolo[4,5-c]pyridin-2-yl)carbamate (43, 200 mg, 699.91 mmol) and o-tolylboronic acid (475.79 mg, 3.50 mmol) in DMF (2.0 mL) and water (0.1 mL). The reaction mixture was stirred at 100° C. for 3 hours by in a microwave. The mixture was filtered. Water (5.0 mL) was added to the mixture that was then extracted three times with EtOAc (5.0 mL), dried over anhydrous sodium sulfate, filtered and concentrated. The residue was purified by silica gel column chromatography to provide the free base of the title compound tert-butyl (6-(o-tolyl)thiazolo[4,5-c]pyridin-2-yl)carbamate (44, 700 mg, 53% yield). MS (m/z): 342.1 [M+H]⁺.

### Example 19: Preparation of 6-(o-tolyl)thiazolo[4,5-c]pyridin-2-amine (45)

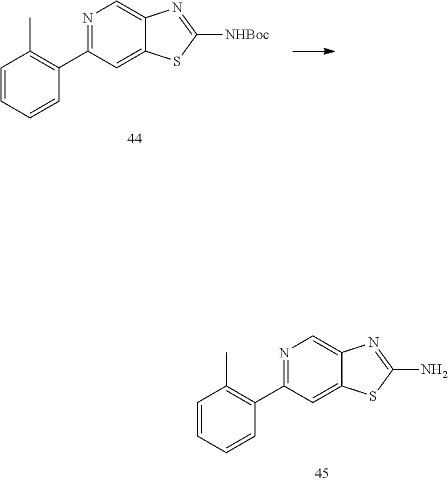 [SMILES: Cc1ccccc1-c1cc2sc(NC(=O)OC(C)(C)C)nc2cn1 ; Cc1ccccc1-c1cc2sc(N)nc2cn1]

tert-Butyl (6-(o-tolyl)thiazolo[4,5-c]pyridin-2-yl)carbamate (44, 650 mg, 1.90 mmol) was dissolved in TFA (7.70 g, 67.53 mmol, 5.0 mL) and DCM (5.0 mL). The solution was stirred at 25° C. for 2 hours. The mixture was concentrated and a sat. NaHCO₃ solution was added to the residue until the pH=7-8. The reaction mixtures was extracted three times with EtOAc (5.0 mL), dried over anhydrous sodium sulfate, filtered and concentrated to provide the free base of the title compound 6-(o-tolyl)thiazolo[4,5-c]pyridin-2-amine (45, 400 mg, crude) as a yellow solid.

### Example 20: Preparation of 1-methyl-N-(6-(o-tolyl)thiazolo[4,5-c]pyridin-2-yl)-1H-pyrazole-5-carboxamide (Product 42)

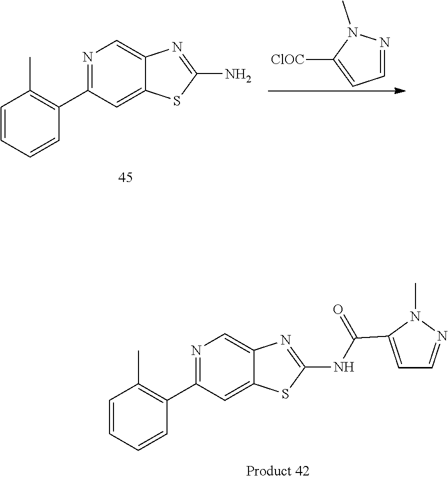

Trieathylamine (125.80 mg, 1.24 mmol, 173.04 mL), DMAP (101.26 mg, 828.81 mmol) and then 1-methyl-1H-pyrazole-5-carbonyl chloride (599.06 mg, 4.14 mmol) were added in sequence to a solution of 6-(o-tolyl)thiazolo[4,5-c]pyridin-2-amine (45, 200 mg, 828.81 mmol) in THF (5.0 mL) at 0° C. The reaction mixtures was stirred at 25° C. for 5 hours. Water (20 ml) was added and the reaction mixture was extracted three times with EtOAc (20 mL). The combined organic layers were washed three times with brine (10 mL), dried over anhydrous sodium sulfate, filtered and concentrated. The residue was purified by silica gel column chromatography to provide the free base of the title compound 1-methyl-N-(6-(o-tolyl)thiazolo[4,5-c]pyridin-2-yl)-1H-pyrazole-5-carboxamide (Product 42, 34 mg, 11% yield). ¹H-NMR (DMSO-d₆, 400 MHz): δ_{H} 13.14 (1H, s), 9.12 (1H, s), 8.23 (1H, d, J=0.8 Hz), 7.61 (1H, d, J=2.4 Hz), 7.46-7.41 (2H, m), 7.34-7.21 (3H, m), 4.18 (3H, s), 2.36 (3H, s); MS (m/z):350.1 [M+H]⁺; purity 97%.

### Example 21: Preparation of 1-methyl-N-(5-(o-tolyl)oxazolo[5,4-b]pyridin-2-yl)-1H-pyrazole-5-carboxamide (Product 43)

### Step 1. Preparation of 3-nitro-6-(o-tolyl) pyridin-2-ol (20)

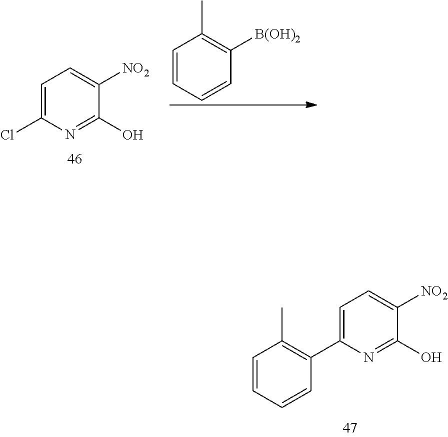

A mixture of 6-chloro-3-nitro-pyridin-2-ol (46, 5.0 g, 28.65 mmol), o-tolylboronic acid (5.84 g, 42.97 mmol), K₂CO₃ (11.88 g, 85.94 mmol), Pd(dppf)Cl₂·CH₂C₁₂ (3.04 g, 3.72 mmol) in a mixture of dioxane (20 mL) and water (10 mL) was degassed and purged with N₂ for 3 times, and then the mixture was stirred at 90° C. for 4 hours under N₂ atmosphere. The crude reaction mixture was adjusted to pH=7 with HCl (1N) aqueous. The aqueous phase was extracted three times with ethyl acetate (50 mL). Then the reaction mixture was concentrated under reduced pressure. The residue was purified by silica gel column chromatography to afford the title compound 3-nitro-6-(o-tolyl) pyridin-2-ol (47, 4.0 g, 60% yield). ¹H-NMR (DMSO-d₆, 400 MHz): δH 12.96 (1H, s), 8.47 (1H, d, J=8 Hz), 7.45 (1H, t, J=8 Hz), 7.21-7.37 (3H, m), 6.37 (1H, d, J=8 Hz), 2.30 (3H, s).

### Step 2. Preparation of 3-amino-6-(o-tolyl) pyridin-2-ol (48)

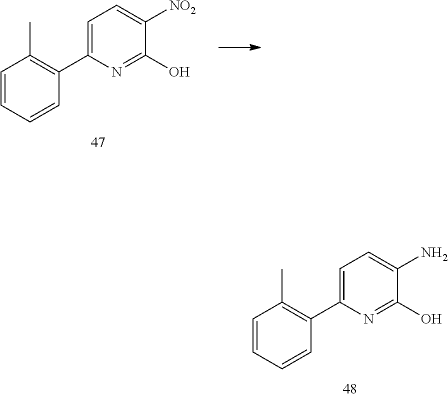 [SMILES: Cc1ccccc1-c1ccc([N+](=O)[O-])c(O)n1 ; Cc1ccccc1-c1ccc(N)c(O)n1]

Pd/C (0.8 g, 10% purity) was added to a mixture of 3-nitro-6-(o-tolyl) pyridin-2-ol (20, 4.0 g, 17.37 mmol) in THF (30 mL). The mixture was degassed and purged with H₂ for three times. The mixture was stirred under H₂ (15 psi) for 5 hours at 25° C. The mixture is filtered through celite and the solvent is removed under reduced pressure to give a residue. The residue was purified by silica gel column chromatography to provide the title compound 3-amino-6-(o-tolyl) pyridin-2-ol (21, 1.88 g, 54% yield). ¹H-NMR (DMSO-d₆, 400 MHz): δH 11.43 (1H, s), 7.20-7.27 (4H, m), 6.51 (1H, d, J=7.2 Hz), 5.93 (1H, d, J=7.2 Hz), 5.06 (2H, s), 2.25 (3H, s);

### Step 3. Preparation of 5-(o-tolyl) oxazolo [5, 4-b] pyridine-2-thiol (49)

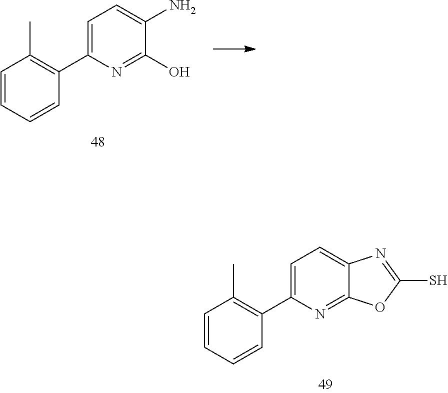 [SMILES: Cc1ccccc1-c1ccc(N)c(O)n1 ; Cc1ccccc1-c1ccc2nc(S)oc2n1]

Thiocarbonyl dichloride (689.08 mg, 459.38 mL) was added at 0° C. to a mixture of 3-amino-6-(o-tolyl)pyridin-2-ol (48, 1 g, 4.99 mmol) in THF (5.0 mL). The mixture was stirred at 0° C. for 5 hours under N₂ atmosphere. The reaction mixture was concentrated under reduced pressure. The residue was purified by silica gel column chromatography to afford the title compound 5-(o-tolyl) oxazolo [5, 4-b]pyridine-2-thiol (49, 0.7 g, 58% yield). ¹H-NMR (DMSO-d₆, 400 MHz): δ_{H} 7.72 (1H, d, J=7.6 Hz), 7.47 (1H, d, J=8 Hz), 7.39 (1H, d, J=7.2 Hz), 7.29-7.35 (4H, m), 2.34 (3H, s).

### Step 4. Preparation of 2-(methylthio)-5-(o-tolyl)oxazolo[5,4-b]pyridine (50)

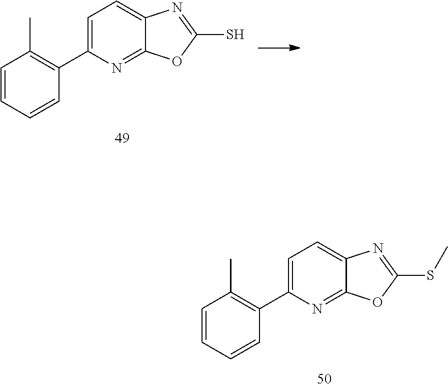 [SMILES: Cc1ccccc1-c1ccc2nc(S)oc2n1 ; CSc1nc2ccc(-c3ccccc3C)nc2o1]

A mixture of 5-(o-tolyl)oxazolo[5,4-b]pyridine-2-thiol (49, 0.84 g, 3.47 mmol), K₂CO₃ (479.15 mg, 3.47 mmol), MeI (738.12 mg, 5.20 mmol, 323.74 mL) in THF (10 mL) was degassed and purged with N₂ for three times. The mixture was stirred at 25° C. for 5 hours under N₂ atmosphere. The reaction mixture was concentrated under reduced pressure. The residue was purified by silica gel column chromatography to afford the title compound 2-(methylthio)-5-(o-tolyl)oxazolo[5,4-b]pyridine (50, 0.84 g, 94% yield).

### Step 5. Preparation of 5-(o-tolyl)oxazolo[5,4-b]pyridin-2-amine (51)

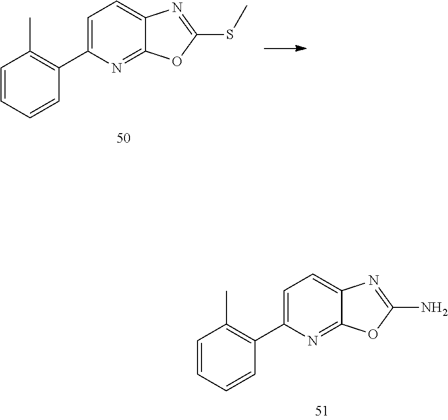 [SMILES: CSc1nc2ccc(-c3ccccc3C)nc2o1 ; Cc1ccccc1-c1ccc2nc(N)oc2n1]

A solution of 2-methylsulfanyl-5-(o-tolyl)oxazolo[5,4-b]pyridine (50, 450 mg, 1.76 mmol) in NH₃. Water (20 mL) was stirred under 50 Psi at 80° C. for 5 hours in a 100 mL of sealed tube. The reaction mixture was concentrated under reduced pressure. The reaction mixture was diluted with water (10 mL) and extracted three times with EtOAc (10 mL). The combined organic layers were dried over anhydrous sodium sulfate, filtered and concentrated under reduced pressure to provide the title compound 5-(o-tolyl)oxazolo[5,4-b]pyridin-2-amine (51, 146 mg, 37% yield) that was used in the next step without any further purification. ¹H-NMR (DMSO-d₆, 400 MHz): δH 7.78 (1H, s), 7.60 (1H, d, J=7.6 Hz), 7.36-7.38 (1H, m), 7.26-7.28 (3H, m), 2.33 (3H, s).

### Step 6. Preparation of 1-methyl-N-(5-(o-tolyl)oxazolo[5,4-b]pyridin-2-yl)-1H-pyrazole-5-carboxamide (Product 43)

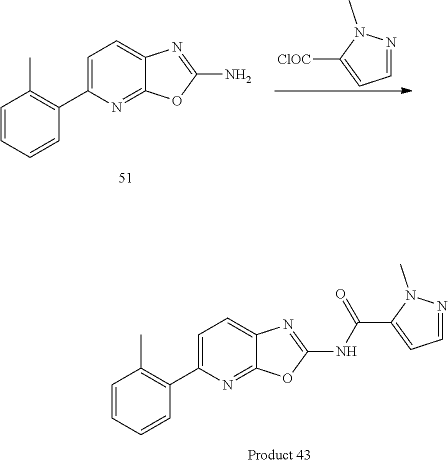

A mixture of 2-methylpyrazole-3-carbonyl chloride (100 mg, 691.76 mmol) in DCM (5.0 mL) was added to a mixture of 5-(o-tolyl)oxazolo[5,4-b]pyridin-2-amine (51, 146 mg, 648.18 mmol), TEA (163.97 mg, 1.62 mmol, 225.55 mL) and DMAP (79.19 mg, 648.18 mmol) in THF (5 mL). The reaction mixture was degassed, purged with N₂ for three times and stirred at 60° C. for 10 hours under N₂ atmosphere. The combined organic phase was washed twice with brine (10 mL), dried with anhydrous sodium sulfate, filtered and concentrated under reduced. The residue was purified by prep-HPLC to afford the title compound 1-methyl-N-(5-(o-tolyl)oxazolo[5,4-b]pyridin-2-yl)-1H-pyrazole-5-carboxamide (Product 43, 52 mg, 24% yield). ¹H-NMR (DMSO-d₆, 400 MHz): δH 8.05 (1H, d, J=8 Hz), 7.53-7.57 (2H, m), 7.45 (1H, d, J=6.8 Hz), 7.30-7.35 (3H, m), 7.20 (1H, s), 4.15 (3H, s), 2.37 (3H, s); MS (m/z):334.2 [M+H]⁺; purity 96%.

### Example 22: Preparation of tert-butyl 6-(1-methyl-1H-pyrazole-5-carboxamido)-2-(2-trifluoromethyl)phenyl)-1H-pyrrolo[3,2-b]pyridine-1-carboxylate (Product 44)

### Step 1. Preparation of tert-butyl 6-bromo-1H-pyrrolo[3,2-b]pyridine-1-carboxylate (53)

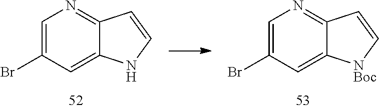 [SMILES: Brc1cnc2cc[nH]c2c1 ; CC(C)(C)OC(=O)n1ccc2ncc(Br)cc21]

A 500 mL round bottom flask was charged with 6-chloro-1H-pyrrolo[3,2-b]pyridine (52, 20 g, 76.1 mmol) DMAP (930 mg, 7.6 mmol) and 350 mL of ACN. Boc₂O (21.0 mL, 91.4 mmol) was then added dropwise upon stirring and the reaction mixture was stirred overnight at room temperature. The reaction was quenched by addition of 100 mL of water and the aqueous phase was extracted three times with 150 mL of EtOAc. The combined organic phase was washed with brine and dried over anhydrous magnesium sulfate. The solids were removed by filtration and the solvent was removed in vacuo. The crude material was purified by silica flash chromatography to afford tert-butyl 6-bromo-1H-pyrrolo[3,2-b]pyridine-1-carboxylate (53, 20.36 g, 90% yield). ¹H-NMR (CHCl₃-d, 400 MHz): δ_{H} 1.67 (8H, s), 6.73 (1H, d, J=3.8 Hz), 7.78 (1H, s), 8.56 (1H, d, J=1.8 Hz). MS (m/z): 297.00 [M+H]⁺.

Intermediates 1 Reported in Table 6 were Prepared According to the Above Step:

TABLE 6

| | | | | MS (m/z) |
|---|---|---|---|---|
| Inter- | | | | [M + H]⁺; |
| mediate | | | | Purity |
| name | Reagent | Structure and name | ¹H-NMR | (%) |
| Inter- mediate 1A | 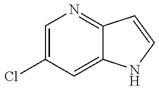 | 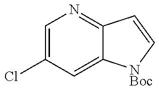 [SMILES: CC(C)(C)OC(=O)n1ccc2ncc(Cl)cc21] | (CHCl₃-d, 400 MHz): δ_{H} 1.68 (10H, s), 6.74 (1H, d, J = 3.8 Hz), 7.80 (1H, s), 8.40 (1H, s), 8.48 (1H, d, J = 2.2 Hz). | 253.10; NA |
| Inter- mediate 1B | 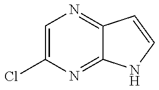 | 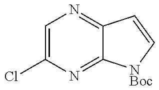 [SMILES: CC(C)(C)OC(=O)n1ccc2ncc(Cl)nc21] | (400 MHz, CDCl₃): δ_{H} 8.51 (1 H, s), 7.94 (1 H, d, J 4.2), 6.76 (1 H, d, J 4.2), 1.69 (9 H, s). | 254.10; NA |
| Inter- mediate 1C | 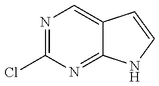 | 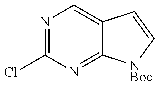 [SMILES: CC(C)(C)OC(=O)n1ccc2cnc(Cl)nc21] | NA | 254.10; NA |
| Inter- mediate 1D | 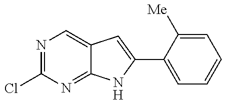 [SMILES: Cc1ccccc1-c1cc2cnc(Cl)nc2[nH]1] | 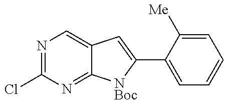 [SMILES: Cc1ccccc1-c1cc2cnc(Cl)nc2n1C(=O)OC(C)(C)C] | (400 MHz, CDCl₃): δ_{H} 8.83 (1 H, s), 7.39- 7.33 (1 H, m), 7.30-7.23 (3 H, m), 6.47 (1 H, s), 2.19 (3 H, s), 1.26 (9 H, s). | 344.20; NA |

### Step 2. Preparation of tert-butyl 6-bromo-2-(4,4,5,5-tetramethyl-1,3,2-dioxaborolan-2-yl)-1H-pyrrolo[3,2-b]pyridine-1-carboxylate (55)

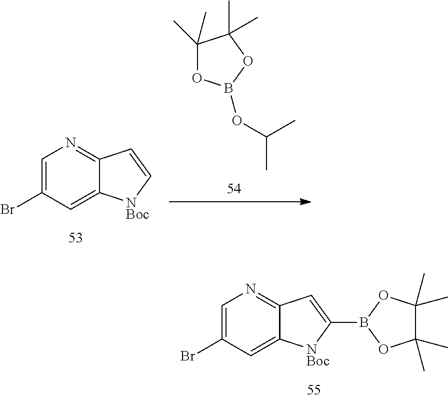 [SMILES: CC(C)(C)OC(=O)n1ccc2ncc(Br)cc21 ; CC(C)OB1OC(C)(C)C(C)(C)O1 ; CC(C)(C)OC(=O)n1c(B2OC(C)(C)C(C)(C)O2)cc2ncc(Br)cc21]

A flame dried 500 mL round bottom flask was charged with tert-butyl 6-bromo-1H-pyrrolo[3,2-b]pyridine-1-carboxylate (53, 15 g, 50.5 mmol) and 2-isopropoxy-4,4,5,5-tetramethyl-1,3,2-dioxaborolane (54, 14.09 g, 75.7 mmol) and dissolved in 90.0 mL THF. The reaction mixture was cooled to 0° C. and freshly prepared LDA (from 8.84 mL diisopropylamine and 25.2 mL 2.5 M butyllithium (in hexanes) in 30 mL THF) was added dropwise over 1 hour. The reaction mixture was then stirred for 1 hour at 0° C. until TLC showed full conversion of the SM. The reaction was quenched with 180 mL of water/1 N HCl (1:1) and stirred until two clear phases appear. The organic phase was collected and the aqueous phase was extracted twice with EtOAc (90 mL). The combined organic phase was washed with brine and dried over MgSO₄. The solids were removed by filtration and the solvent was removed in vacuo. The crude material was dissolved in DCM and treated with activated charcoal (20 g). The solids were removed by filtration on Celite and the solvent was removed in vacuo to afford tert-butyl 6-bromo-2-(4,4,5,5-tetramethyl-1,3,2-dioxaborolan-2-yl)-1H-pyrrolo[3,2-b]pyridine-1-carboxylate (55, 18.61 g, 97% yield). ¹H-NMR (CHCl₃-d, 400 MHz): δ_{H} 1.41 (12H, s), 1.70 (9H, s), 6.98 (1H, s), 8.32 (1H, s), 8.54 (1H, d, J=2.0 Hz). MS (m/z): 424.24 [M+H]⁺.

Intermediates 2 Reported in Table 7 were Prepared According to the Above Step:

TABLE 7

| | | | | MS (m/z) |
|---|---|---|---|---|
| | | | | [M + H]⁺; |
| Intermediate | | | | Purity |
| name | Reagent | Structure and name | ¹H-NMR | (%) |
| Intermediate 2A | 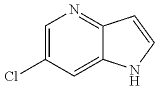 | 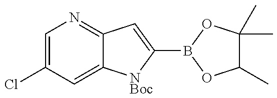 [SMILES: CC1OB(c2cc3ncc(Cl)cc3n2C(=O)OC(C)(C)C)OC1(C)C] | (CHCl₃-d, 400 MHz): δ_{H} 1.68 (10H, s), 7.03 (1H,d, J = 3.8 Hz), 7.80 (1H, s), 8.40 (1H, s). | 379.0; NA |
| Intermediate 2B | 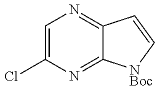 [SMILES: CC(C)(C)OC(=O)n1ccc2ncc(Cl)nc21] | 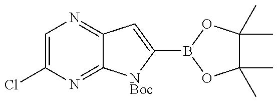 [SMILES: CC(C)(C)OC(=O)n1c(B2OC(C)(C)C(C)(C)O2)cc2ncc(Cl)nc21] | NA | 379.1 |
| Intermediate 2C | 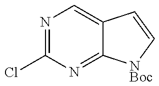 [SMILES: CC(C)(C)OC(=O)n1ccc2cnc(Cl)nc21] | 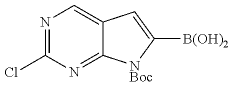 [SMILES: CC(C)(C)OC(=O)n1c(B(O)O)cc2cnc(Cl)nc21] | NA | 298.2 |

### Step 3. Preparation of tert-butyl 6-brom o-2-(2-(trifluoromethyl)phenyl)-1H-pyrrolo[3,2-b]pyridine-1-carboxylate (57)

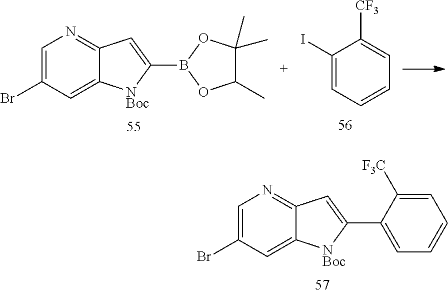

A flame dried 200 mL pressure vial was charged with 2-iodobenzotrifluoride (56, 5 g, 18.5 mmol), the tert-butyl 6-bromo-2-(4,4,5,5-tetramethyl-1,3,2-dioxaborolan-2-yl)-1H-pyrrolo[3,2-b]pyridine-1-carboxylate (55, 8.3 g, 20.3 mmol), P(tBu)₃Pd(crotyl)Cl (370 mg, 5 mol %) and Na₂CO₃ (5.9 g, 55.6 mmol). The reaction vial was purged with argon three times then toluene (90 mL) and water (30 mL) were added and argon was bubbled for 10 min. The vial was sealed with a Teflon screw cap and the reaction was plugged in a pre-heated bath at 75° C. for 2 h. The reaction was cooled down to room temperature and water (30 mL) was added. The two phases were separated, and the aqueous phase was back extracted with three times with 100 mL of EtOAc. The combined organic layers were washed with brine and dried over anhydrous magnesium sulfate. The solids were removed by filtration and the volatiles were removed in vacuo. The crude material was purified by silica gel flash chromatography to afford tert-butyl 6-bromo-2-(2-(trifluoromethyl)phenyl)-1H-pyrrolo[3,2-b]pyridine-1-carboxylate (57, 5.8 g, 71% yield). ¹H-NMR (CHCl₃-d, 400 MHz): δ_{H} 1.18 (9H, s), 6.73-6.71 (1H, m), 7.42 (1H, d, J=7.4 Hz), 7.62-7.53 (2H, m), 7.75 (1H, d, J=7.8 Hz), 8.59 (1H, s), 8.73 (1H, d, J=2.0 Hz); MS (m/z): 441.05 [M+H]⁺.

Intermediates 3 Reported in Table 8 were Prepared According to the Above Step:

TABLE 8

| | | | | | MS |
|---|---|---|---|---|---|
| | | | | | (m/z) |
| Inter- | | | | | [M + |
| me- | | | | | H]⁺; |
| diate | | | | | Purity |
| name | Reagent | Reagent | Structure and name | ¹H-NMR | (%) |
| Inter- me- diate 3A | 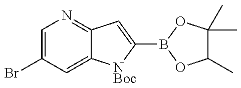 [SMILES: CC1OB(c2cc3ncc(Br)cc3n2C(=O)OC(C)(C)C)OC1(C)C] | 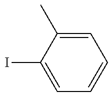 | 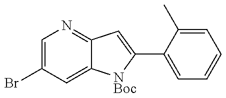 [SMILES: Cc1ccccc1-c1cc2ncc(Br)cc2n1C(=O)OC(C)(C)C] | (CHCl₃-d, 400 MHz): δ_{H} 1.22 (9H, s), 2.17 (3H, s), 6.63 (1H, s), 7.33-7.24 (4H, m), 8.59 (1H, d, J = 2.1 Hz), 8.71 (1H, d, J = 2.0 Hz). | 387.10, 389.10 NA |
| Inter- me- diate 3B | 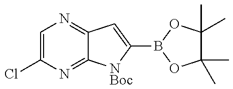 [SMILES: CC(C)(C)OC(=O)n1c(B2OC(C)(C)C(C)(C)O2)cc2ncc(Cl)nc21] | 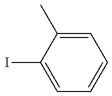 | 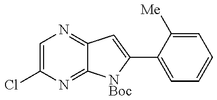 [SMILES: Cc1ccccc1-c1cc2ncc(Cl)nc2n1C(=O)OC(C)(C)C] | (400 MHz, CDCl₃): δ_{H} 8.53 (1 H, s), 7.40-7.33 (1 H, m), 7.30-7.27 (3 H, m), 6.65 (1 H, s), 2.20 (3 H, s), 1.26 (9 H, s). | 344.10; NA |
| Inter- me- diate 3C | 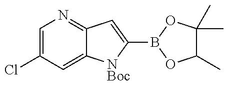 [SMILES: CC1OB(c2cc3ncc(Cl)cc3n2C(=O)OC(C)(C)C)OC1(C)C] | 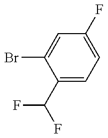 [SMILES: Fc1ccc(C(F)F)c(Br)c1] | 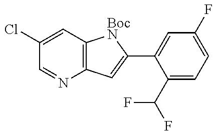 [SMILES: CC(C)(C)OC(=O)n1c(-c2cc(F)ccc2C(F)F)cc2ncc(Cl)cc21] | NA | 397.10; NA |
| Inter- me- diate 3D¹ | 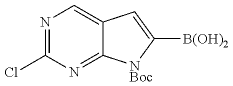 [SMILES: CC(C)(C)OC(=O)n1c(B(O)O)cc2cnc(Cl)nc21] | 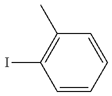 | 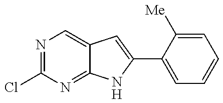 [SMILES: Cc1ccccc1-c1cc2cnc(Cl)nc2[nH]1] | NA | 244.10; NA |

¹The reaction was performed using K₂CO₃ as base, dioxane/water (2/1) as solvent, bis(diphenylphosphino)ferrocene]dichloropalladium(II) as catalyst and by heating at 90° C. for 2 hours.

### Example 23: Preparation of 1-methyl-1H-pyrazole-5-carboxamide (60)

### Step 1. Preparation of methyl 1-methyl-1H-pyrazole-5-carboxylate (59)

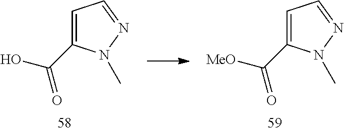

To a stirred solution of 1-methyl-1H-pyrazole-5-carboxylic acid (58, 1 g, 7.9 mmol) in methanol (16 mL) was added sulfuric acid (0.42 mL, 7.9 mmol) and the reaction mixture was refluxed for 20 hours. Volatiles were removed under vacuum and the residue was dissolved with EtOAc. The solution was washed with sat. NaHCO₃, brine sequentially, dried over anhydrous sodium sulfate and concentrated to provide the title compound methyl 1-methyl-1H-pyrazole-5-carboxylate (59, 0.76 g, 68% yield)¹H-NMR (CHCl₃-d, 400 MHz): δ_{H} 3.88 (3H, s), 4.19 (3H, s), 6.83 (1H, d, J=2.0 Hz), 7.46 (1H, d, J=2.0 Hz)). MS (m/z): 141.1 [M+H]⁺.

### Step 2. Preparation of 1-methyl-1H-pyrazole-5-carboxamide (60)

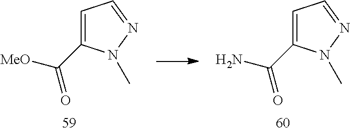

Methyl 1-methyl-1H-pyrazole-5-carboxylate (59, 0.75 g, 5.4 mmol) was stirred in ammonium hydroxide (7.5 mL) at room temperature for 1 h. The product was extracted with 10% IPA in chloroform, dried over anhydrous magnesium sulfate and the solvents were removed in vacuum to provide the title compound 1-methyl-1H-pyrazole-5-carboxamide (60, 0.5 g, 75% yield). ¹H-NMR (CHCl₃-d, 400 MHz): δ_{H} 4.18 (3H, s), 5.91 (2H, br s), 6.56 (1H, d, J=2.1 Hz), 7.45 (1H, d, J=2.1 Hz). MS (m/z): 126.1 [M+H]⁺.

### Example 24: Preparation of 1-methyl-1H-1,2,4-triazole-5-carboxamide (64)

### Step 1. Preparation of methyl 1-methyl-1H-1,2,4-triazole-5-carboxylate (63)

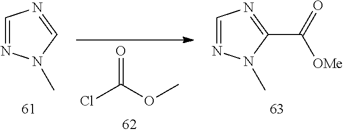

A solution of triazole (61, 3 g, 36.0 mmol) in anhydrous THF (120 ml) was cooled to −78° C. under nitrogen and treated with n-butyl lithium (15 mL of 2.5 M solution in hexane, 38 mmol) over a 15 minute period. The off white solution was stirred for 30 min at −78° C., and brought to 0° C. and stirred for 20 min. The reaction mixture was cooled to −78° C., treated with methyl chloroformate (62, 11 ml, 144 mmol), stirred at −78° C. and allowed to gradually warm to room temperature overnight. The yellow reaction mixture was quenched with water (10 mL), followed by addition of ethyl acetate. The organic layer was collected and washed with brine and dried over anhydrous magnesium sulfate, filtered and concentrated in vacuo. Purification of the residue by silica gel chromatography afforded the title compound methyl 1-methyl-1H-1,2,4-triazole-5-carboxylate (63, 3.2 g, 63% yield). ¹H-NMR (CHCl₃-d, 400 MHz): δ_{H} 4.00 (3H, s), 4.24 (3H, s), 7.95 (1H, s); MS (m/z): 142.1 [M+H]⁺.

### Step 2. Preparation of 1-methyl-1H-1,2,4-triazole-5-carboxamide (64)

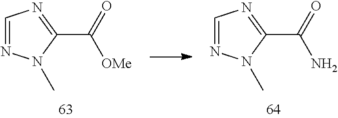

In a sealed tube, a mixture of methyl 1-methyl-1H-1,2,4-triazole-5-carboxylate (63, 1.1 g, 7.79 mmol) and ammonia in methanol (11 mL, 77.94 mmol) was stirred at 65° C. for 15 hours. Following completion of the reaction, the ammonia/methanol solution was evaporated under vacuo to give solid. The solid was triturated with hexane/diethyl ether (8:2) and filtered to afford 1-methyl-1H-1,2,4-triazole-5-carboxamide (64, 0.94 g, 96% yield). ¹H-NMR (DMSO-d6, 400 MHz): δ_{H} 4.10 (3H, s), 7.90 (1H, s), 8.01 (1H, s), 8.12 (1H, s); MS (m/z): 127.1 [M+H]⁺. Step 4. Preparation of 1-methyl-N-(2-(2-(trifluoromethyl)phenyl)-1H-pyrrolo[3,2-b]pyridin-6-yl)-1H-pyrazole-5-carboxamide (Product 44).

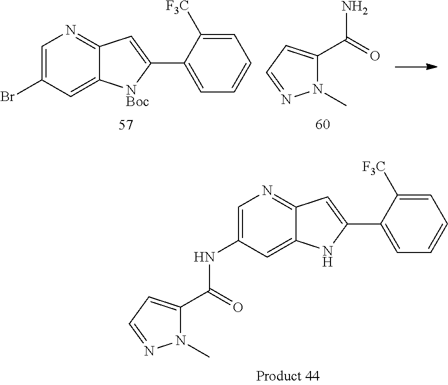

A flame dried 10 mL pressure vial was charged with tert-butyl 6-bromo-2-(2-(trifluoromethyl)phenyl)-1H-pyrrolo[3,2-b]pyridine-1-carboxylate (57, 200 mg, 0.45 mmol), 1-methyl-1H-pyrazole-5-carboxamide(60, 113 mg, 0.91 mmol), [XantPhos Pd(allyl)]Cl (17 mg, 0.02 mmol, 5 mol %), Cs₂CO₃ (221 mg, 0.68 mmol) and dioxane (4.5 mL). The reaction mixture was degassed 10 min with argon and the reaction vial was sealed with a Teflon screw cap. The reaction vessel was plugged in a pre-heated bath at 110° C. and stirred for 16 h. The reaction was cooled to room temperature and water (20 mL) was added. The phases were separated, and the aqueous phase was extracted three times with 50 mL of EtOAc. The combined organic layers were washed with brine and dried over anhydrous magnesium sulfate. The solids were removed by filtration and the volatiles were removed in vacuo. The crude material was dissolved in DCM (5.0 mL) and the reaction was cooled to 0° C. and TFA (5.0 mL) was added dropwise. The reaction mixture was then allowed to warm to room temperature and stirred until full completion. Toluene (10 mL) was added and the solvents were removed in vacuo. The crude material was then purified by reverse phase chromatography to afford 1-methyl-N-(2-(2-(trifluoromethyl)phenyl)-1H-pyrrolo[3,2-b]pyridin-6-yl)-1H-pyrazole-5-carboxamide Product 44 (240 mg, 58%). ¹H-NMR (DMSO-d₆, 400 MHz): δ_{H} 4.11 (3H, s), 6.65 (1H, s), 7.11 (1H, d, J=2.1 Hz), 7.55 (1H, d, J=2.0 Hz), 7.73-7.66 (2H, m), 7.81 (1H, t, J=7.6 Hz), 7.91 (1H, d, J=7.9 Hz), 8.33 (1H, s), 8.58 (1H, d, J=2.2 Hz), 10.39 (1H, s), 11.65 (1H, s); MS (m/z): 386.1 [M+H]⁺; >99% purity.

Final compounds reported in Table 9 were prepared according to step 4 for the synthesis of Product 44 using the corresponding carboxamide.

TABLE 9

| | | | | | MS (m/z) |
|---|---|---|---|---|---|
| Final | | | | | [M + H]⁺; |
| compound | Reagent | Reagent | Structure and name | ¹H-NMR | Purity (%) |
| Product 45 | 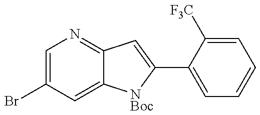 [SMILES: CC(C)(C)OC(=O)n1c(-c2ccccc2C(F)(F)F)cc2ncc(Br)cc21] | 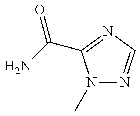 | 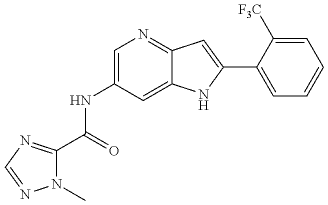 [SMILES: Cn1ncnc1C(=O)Nc1cnc2cc(-c3ccccc3C(F)(F)F)[nH]c2c1] | (DMSO-d₆, 400 MHz): δ_{H} 4.20 (3H, s), 6.65 (1H, s), 7.73-7.66 (2H, m), 7.81 (1H, t, J = 7.6 Hz), 7.91 (1H, d, J = 7.9 Hz), 8.17 (1H, s), 8.37 (1H, s), 8.70 (1H, d, J = 2.2 Hz). | 387.10; 99.0 |
| Product 46 | 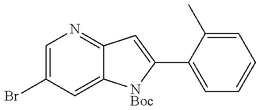 [SMILES: Cc1ccccc1-c1cc2ncc(Br)cc2n1C(=O)OC(C)(C)C] | 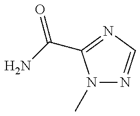 | 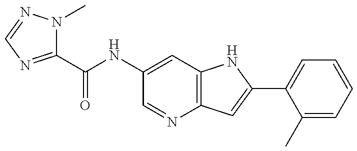 [SMILES: Cc1ccccc1-c1cc2ncc(NC(=O)c3ncnn3C)cc2[nH]1] | (DMSO-d₆, 400 MHz): δ_{H} 3.27 (3H, s), 4.20 (3H, s), 6.69 (1H, s), 7.35-7.30 (3H, m), 7.56- 7.54 (1H, m), 8.17 (1H, s), 8.34 (1H, s), 8.67 (1H, d, J = 2.2 Hz). | 333.10; 98.0 |
| Product 47 | 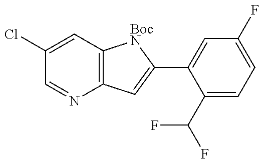 [SMILES: CC(C)(C)OC(=O)n1c(-c2cc(F)ccc2C(F)F)cc2ncc(Cl)cc21] | 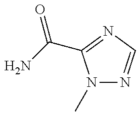 | 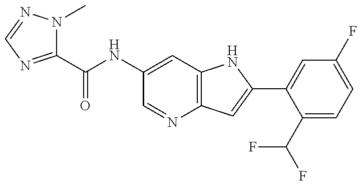 [SMILES: Cn1ncnc1C(=O)Nc1cnc2cc(-c3cc(F)ccc3C(F)F)[nH]c2c1] | (DMSO-d₆, 400 MHz): δ_{H} 4.21 (3H, s), 6.75 (1H, s), 7.17 (1H, t, J = 54.3 Hz), 7.46 (1H, t, J = 8.5 Hz), 7.59 (1H, d, J = 9.9 Hz), 7.88 (1H, t, J = 7.2 Hz), 8.18 (1H, s), 8.41 (1H, s), 8.76 (1H, d, J = 2.2 Hz). | 387.1; 99.0 |
| Product 48 | 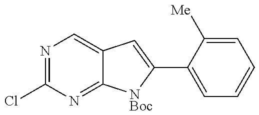 [SMILES: Cc1ccccc1-c1cc2cnc(Cl)nc2n1C(=O)OC(C)(C)C] | 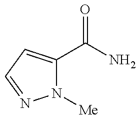 | 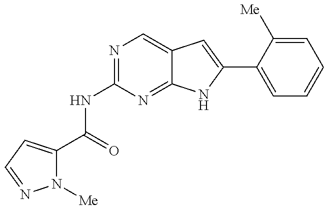 [SMILES: Cc1ccccc1-c1cc2cnc(NC(=O)c3ccnn3C)nc2[nH]1] | (400 MHz, DMSO): δ_{H} 10.84 (1 H, s), 8.93 (1 H, s), 7.57-7.54 (1 H, m), 7.52 (1 H, d, J 2.0), 7.39-7.30 (3 H, m), 7.19 (2 H, d, J 1.9), 6.68 (1H, s), 4.11 (3 H, s), 2.46 (3 H, s). | 333.1; 99.7 |

### Example 25: Preparation of 1-methyl-N-(2-(o-tolyl)-1H-pyrrolo[3,2-b]pyridin-6-yl)-1H-pyrazole-5-carboxamide (Product 49)

### Step 1. Preparation of tert-butyl 6-(1-methyl-1H-pyrazole-5-carboxamido)-2-(o-tolyl)-1H-pyrrolo[3,2-b]pyridine-1-carboxylate (61)

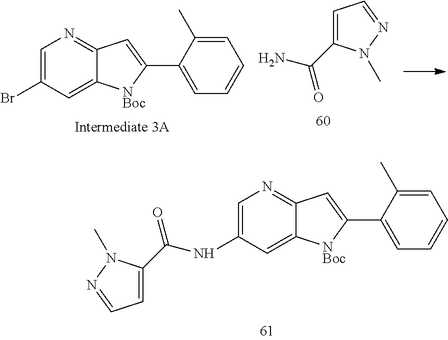

In a flame-dried microwave vial, tert-butyl 6-bromo-2-(o-tolyl)-1H-pyrrolo[3,2-b]pyridine-1-carboxylate (Intermediate 3A, 120 mg, 0.31 mmol), 1-methyl-1H-pyrazole-5-carboxamide (60, 58 mg, 0.47 mmol), tris(dibenzylideneacetone) dipalladium (11 mg, 0.012 mmol), Xantphos (11 mg, 0.019 mmol) and cesium carbonate (152 mg, 0.47 mmol) were suspended in anhydrous dioxane (1.6 mL). The reaction mixture was degassed and refilled with (3 cycles) before heated at 110° C. under argon for 2 hours. After completion, the reaction mixture was cooled down to room temperature, diluted with EtOAc, and filtered through a celite pad. The filtrate was washed with water, brine, and dried over sodium sulfate. Solvents were then removed and the residue was purified by silica chromatography to provide the titled compound (61, 110 mg, 0.25 mmol, 82% yield) ¹H NMR (CHCl₃-d, 400 MHz): δ_{H} 1.25 (9H, s), 2.19 (3H, s), 4.19 (1H, s), 4.26 (3H, s), 6.65 (1H, s), 6.72 (1H, d, J=2.1 Hz), 7.20-7.35 (3H, m), 7.54 (1H, d, J=2.1 Hz), 7.82 (1H, s), 8.65 (1H, d, J=2.3 Hz), 8.97 (1H, d, J=2.2 Hz); MS (m/z): 432.2 [M+H]⁺.

### Step 4. Preparation of 1-methyl-N-(2-(o-tolyl)-1H-pyrrolo[3,2-b]pyridin-6-yl)-1H-pyrazole-5-carboxamide (Product 49)

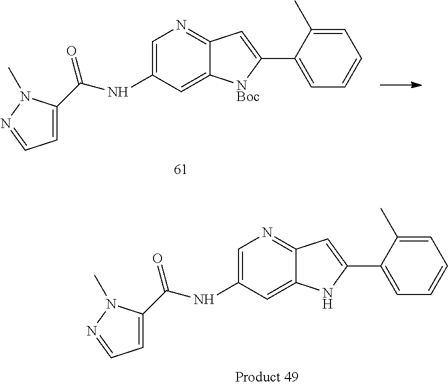 [SMILES: Cc1ccccc1-c1cc2ncc(NC(=O)c3ccnn3C)cc2n1C(=O)OC(C)(C)C ; Cc1ccccc1-c1cc2ncc(NC(=O)c3ccnn3C)cc2[nH]1]

tert-Butyl 6-(1-methyl-1H-pyrazole-5-carboxamido)-2-(o-tolyl)-1H-pyrrolo[3,2-b]pyridine-1-carboxylate (61, 110 mg, 0.25 mmol) was dissolved in 1.3 mL anhydrous DCM and 0.13 mL of TFA were added. The resulting solution was kept stirring at room temperature until complete consumption of starting material. Solvents were then removed under vacuum and the residue was taken up in ethyl acetate. The solution was washed with sat. NaHCO₃, brine, dried over anhydrous sodium sulfate and concentrated. The resulting residue was purified by silica gel column chromatography to provide the title compound 1-methyl-N-(2-(o-tolyl)-1H-pyrrolo[3,2-b]pyridin-6-yl)-1H-pyrazole-5-carboxamide (Product 49, 33 mg, 39% yield). ¹H NMR (CH₃OH-d₄, 400 MHz): δ_{H} 2.49 (3H, s), 4.18 (3H, s), 6.66 (1H, s), 7.02 (1H, d, J=2.1 Hz), 7.35-7.29 (3H, m), 7.54-7.52 (2H, m), 8.39 (1H, s), 8.47 (1H, s); MS (m/z): 332.2 [M+1-1]⁺; purity 97%.

### Example 26: Preparation of 1-methyl-N-(6-(o-tolyl)-5H-pyrrolo[2,3-b]pyrazin-3-yl)-1H-pyrazole-5-carboxamide (Product 50)

### Step 1. Preparation of 3-chloro-6-(o-tolyl)-5H-pyrrolo[2,3-b]pyrazine (64)

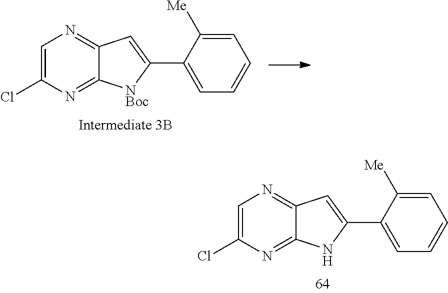 [SMILES: Cc1ccccc1-c1cc2ncc(Cl)nc2n1C(=O)OC(C)(C)C ; Cc1ccccc1-c1cc2ncc(Cl)nc2[nH]1]

A 100 mL round bottom flask was charged with tert-butyl 3-chloro-6-(o-tolyl)-5H-pyrrolo[2,3-b]pyrazine-5-carboxylate (Intermediate 3B, 391 mg, 1.13 mmol), triethylsilane (0.54 mL, 3.41 mmol, 3 equiv.) and 6 mL of DCM. The reaction was cooled to 0° C. and TFA (0.87 mL, 11.3 mmol, 10 equiv.) was added dropwise upon stirring. The reaction was warmed to rt and followed by TLC. The reaction was cooled to 0° C. and was quenched by addition of 10 mL of Na₂CO₃ sat. The aqueous phase was extracted three times with 10 mL of EtOAc. The combined organic phase was washed with brine and dried over anhydrous magnesium sulfate. The solids were removed by filtration and the solvent was evaporated in vacuo. The crude material was purified by silica gel column chromatography to afford 3-chloro-6-(o-tolyl)-5H-pyrrolo[2,3-b]pyrazine (64, 223 mg, 81%). (400 MHz, DMSO): δ_{H} 12.51 (1H, bs), 8.47 (1H, d, J 4.1), 7.59-7.55 (1H, m), 7.41-7.33 (3H, m), 6.85 (1H, d, J 1.8), 2.47 (3H, s); MS (m/z): 244.1 [M+H]⁺.

### Step 5. Preparation of 3-chloro-6-(o-tolyl)-5-((2-(trimethylsilyl)ethoxy)methyl)-5H-pyrrolo[2,3-b]pyrazine (65)

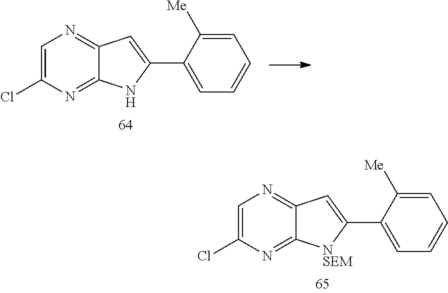 [SMILES: Cc1ccccc1-c1cc2ncc(Cl)nc2[nH]1 ; Cc1ccccc1-c1cc2ncc(Cl)nc2n1COCC[Si](C)(C)C]

A 100 mL round bottom flask was charged with 3-chloro-6-(o-tolyl)-5H-pyrrolo[2,3-b]pyrazine (64, 220 mg, 0.903 mmol, 1.0 equiv.) in anhydrous DMF (4.5 mL). The reaction was cooled to 0° C. and NaH (58 mg, 1.44 mmol, 1.6 equiv.) was added portion wise. The suspension was stirred at 0° C. for 30 min. 2-(Trimethylsilyl)ethoxymethyl chloride (0.24 mL, 1.35 mmol) was then added dropwise. The reaction was warmed to room temperature. The reaction was quenched by addition of 10 mL of water and the aqueous phase was extracted three times with 10 mL of EtOAc. The combined organic phase was washed with brine and dried over anhydrous magnesium sulfate. The solids were removed by filtration and the solvent was evaporated in vacuo. The crude material was purified by silica flash chromatography using a gradient from 100% hexanes to 50% EtOAc in hexanes to afford 3-chloro-6-(o-tolyl)-5-((2-(trimethylsilyl)ethoxy)methyl)-5H-pyrrolo[2,3-b]pyrazine 65 (180 mg, 0.481 mmol, 53%). MS (m/z): 374.1 [M+H]⁺.

### Step 6. Preparation of 1-methyl-N-(6-(o-tolyl)-5-((2-(trimethylsilyl)ethoxy)methyl)-5H -pyrrolo[2,3-b]pyrazin-3-yl)-1H-pyrazole-5-carboxamide (66)

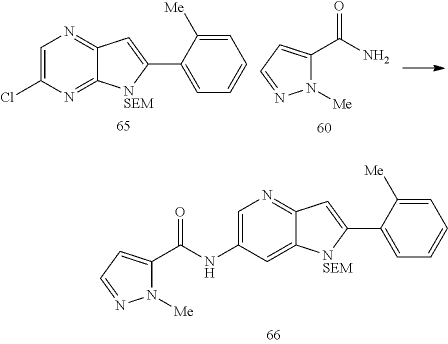

A flame dried 10 mL pressure vial was charged with 3-chloro-6-(o-tolyl)-5-((2-(trimethylsilyl)ethoxy)methyl)-5H-pyrrolo[2,3-b]pyrazine (65, 180 mg, 0.481 mmol), 1-methyl-1H-pyrazole-5-carboxamide (60, 120 mg, 0.962 mmol), CuI (5 mg, 0.024 mmol, 5 mol %), (1R,2R)—N1,N2-dimethylcyclohexane-1,2-diamine (0.004 mL, 0.024 mmol, 5 mol %) and K₂CO₃ (133 mg, 0.962 mmol). The reaction vial was purged with argon three times then dioxane (1 mL) was added and argon was bubbled for 10 min. The vial was sealed with a Teflon screw cap and the reaction was plunged in a pre-heated bath at 170° C. for 16 h. The reaction was cooled down to room temperature and water (5.0 mL) was added. The two phases were separated, and the aqueous phase was back extracted with three times with 10 mL of EtOAc. The combined organic layers were washed with brine and dried over anhydrous magnesium sulfate. The solids were removed by filtration and the volatiles were evaporated in vacuo. The crude material was purified by silica gel column chromatography to afford the title compound 1-methyl-N-(6-(o-tolyl)-5-((2-(trimethylsilyl)ethoxy)methyl)-5H-pyrrolo[2,3-b]pyrazin-3-yl)-1H-pyrazole-5-carboxamide (66, 120 mg, 0.259 mmol, 54%). ¹H-NMR (400 MHz, CDCl₃): δ_{H} 9.54 (1H, s), 8.25 (1H, s), 7.55 (1H, d, J 1.8), 7.44-7.28 (4H, m), 6.78 (1H, d, J 2.0), 6.68 (1H, s), 5.38 (2H, s), 3.37-3.32 (2H, m), 2.25 (3H, s), 0.81-0.75 (2H, m), −0.09 (9H, s); MS (m/z): 463.2 [M+H]⁺.

### Step 7. Preparation of N-(5-(hydroxymethyl)-6-(o-tolyl)-5H-pyrrolo[2,3-b]pyrazin-3-yl)-1-methyl-1H-pyrazole-5-carboxamide (67)

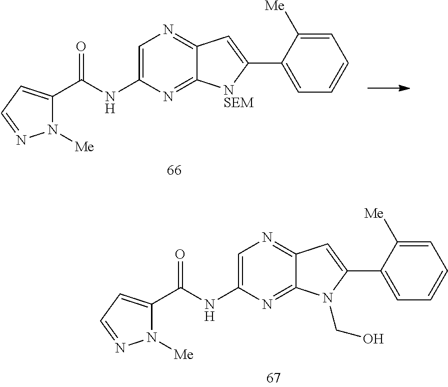 [SMILES: Cc1ccccc1-c1cc2ncc(NC(=O)c3ccnn3C)nc2n1COCC[Si](C)(C)C ; Cc1ccccc1-c1cc2ncc(NC(=O)c3ccnn3C)nc2n1CO]

A 50 mL round bottom flask was charged with 1-methyl-N-(6-(o-tolyl)-5-((2-(trimethylsilyl)ethoxy)methyl)-5H-pyrrolo[2,3-b]pyrazin-3-yl)-1H-pyrazole-5-carboxamide (66, 110 mg, 0.237 mmol), triethylsilane (0.11 mL, 0.713 mmol) and 3.0 mL of DCM. The reaction was cooled to 0° C. and TFA (0.91 mL, 11.9 mmol) was added dropwise upon stirring. The reaction was warmed to room temperature. The reaction mixture was added 3.0 mL of water. The aqueous phase was extracted three times with 10 mL of EtOAc. The combined organic phase was washed with brine and dried over anhydrous magnesium sulfate. The solids were removed by filtration and the solvent was evaporated in vacuo to afford N-(5-(hydroxymethyl)-6-(o-tolyl)-5H-pyrrolo[2,3-b]pyrazin-3-yl)-1-methyl-1H-pyrazole-5-carboxamide 67 that was used in the subsequent reaction without any further purification.

### Step 8. Preparation of 1-methyl-N-(6-(o-tolyl)-5H-pyrrolo[2,3-b]pyrazin-3-yl)-1H -pyrazole-5-carboxamide (Product 50)

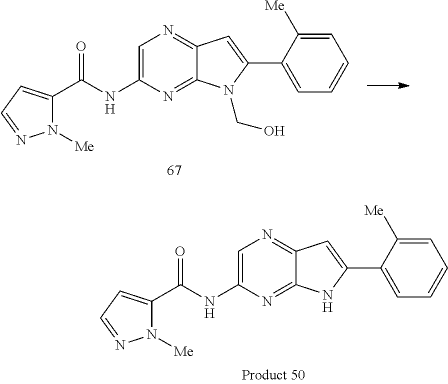 [SMILES: Cc1ccccc1-c1cc2ncc(NC(=O)c3ccnn3C)nc2n1CO ; Cc1ccccc1-c1cc2ncc(NC(=O)c3ccnn3C)nc2[nH]1]

N-(5-(Hydroxymethyl)-6-(o-tolyl)-5H-pyrrolo[2,3-b]pyrazin-3-yl)-1-methyl-1H-pyrazole-5-carboxamide 67 was dissolved in NaHCO₃ sat. (1 mL) and MeOH (1 mL). The reaction was stirred at room temperature overnight. The reaction was added 3.0 mL of water. The aqueous phase was extracted three times with 10 mL of EtOAc. The combined organic phase was washed with brine and dried over anhydrous magnesium sulfate. The solids were removed by filtration and the solvent was evaporated in vacuo. The crude material was then purified by reverse phase chromatography to afford 1-methyl-N-(6-(o-tolyl)-5H-pyrrolo[2,3-b]pyrazin-3-yl)-1H-pyrazole-5-carboxamide (Product 50, 7 mg, 9% yield). ¹H-NMR (400 MHz, DMSO): δ_{H} 12.03 (1H, bs), 10.89 (1H, bs), 9.07 (1H, s), 7.60-7.57 (1H, m), 7.55 (1H, d, J 1.9), 7.41-7.31 (3H, m), 7.27 (1H, d, J 1.9), 6.76 (1H, s), 4.13 (3H, s), 2.47 (3H, s); MS (m/z): 333.1 [M+H]⁺, 93.8% purity.

### Example 27: DRE-Luciferase Reporter Assay

AHR binds to Dioxin Responsive Elements (DRE) upstream of genes that it activates. One measure of AHR activity is activation of a reporter gene, such as luciferase, downstream of one or multiple DRE elements. Luciferase activity will reflect activation and inhibition of AHR in the cells expressing his reporter. 20000 Human HepG2 liver carcinoma—AhR-Lucia reporter cells or Human HT29 colon adenocarcinoma—AhR reporter cells or other cell line with a DRE-luciferase reporter stably transfected were plated in Eagle's Minimal Essential Medium, 10% heat-inactivated FBS, 1×non-essential amino acids Pen-Strep (10,000 U/mL) and Normocin (100 ug/mL) in plates (96-well, 384-well or other plates) and incubated overnight at 37° C. in a CO₂ incubator and treated with and without AhR antagonists at a log dilution starting at 100 uM.

After 1 hr that cells were plated an AHR activating ligand, such as TCDD, kynurenine, ITE (2-(1H-indole-3-ylcarbonyl)-4-thiazolecarboxylic methyl ester), VAF347, BNF (beta-naphthoflavone), FICZ (6-formylindolo(3,2-b) carbazole) or other AHR ligands at their specific EC₅₀ concentration, were added to the cells with or without AHR antagonist.

Cells were incubated for 24 or 48 hours or another time point and then, supernatant was analyzed for determination of luciferase activity as a read-out of the AHR activation or inhibition. Luciferase was measured with the commercial kit QUANTI-Luc™ assay solution kit from Invivogen following the manufacturer's instructions.

The level of luciferase with only agonist ligand added was the maximum signal while the luciferase with no antagonist was the minimum signal. IC₅₀ values were determined as the concentration which inhibits half of the luciferase activity. The IC₅₀ level of luciferase of compounds of the disclosure is reported in Table 10. “A” indicates an IC₅₀ value less than 100 nM, “B” indicates an IC₅₀ between 100 and 500 nM, “C” indicates an IC₅₀ above 500 nM.

TABLE 10

| Name | HEPG2 Luc IC₅₀ (nM) |
|---|---|
| Product 37 | B |
| Product 36 | C |
| Product 23 | C |
| Product 22 | B |
| Product 39 | A |
| Product 21 | C |
| Product 34 | C |
| Product 35 | C |
| Product 20 | A |
| Product 19 | C |
| Product 40 | C |
| Product 14 | A |
| Product 13 | B |
| Product 12 | A |
| Product 10 | C |
| Product 4 | B |
| Product 24 | C |
| Product 33 | B |
| Product 9 | C |
| Product 8 | A |
| Product 31 | B |
| Product 18 | A |
| Product 30 | B |
| Product 32 | C |
| Product 29 | B |
| Product 11 | C |
| Product 28 | C |
| Product 27 | B |
| Product 5 | C |
| Product 25 | A |
| Product 26 | B |
| Product 6 | C |
| Product 17 | C |
| Product 16 | B |
| Product 7 | C |
| Product 3 | B |
| Product 2 | B |
| Product 1 | C |
| Product 15 | A |
| Product 47 | A |
| Product 46 | B |
| Product 45 | A |
| Product 44 | B |
| Product 49 | B |
| Product 50 | C |
| Product 48 | B |
| Product 41 | C |
| Product 42 | C |
| Product 43 | C |

### Example 28: CYP1A1 Gene Expression Assay

Human and mouse colorectal cancer (CRC) cell lines, HT29 and HT26 respectively, American Type Culture Collection (ATCC) are plated in a sterile tissue culture treated 96-well plate (ThermoFisher) at 8.0×10⁵ cells per well, and grown overnight at 37° C., 5% CO₂ in DMEM complete (Gibco) in order to achieve confluence. After the incubation medium is aspirated off the cell monolayers, tissues are then washed with 200 μL of warmed PBS solution, and subsequently 190 μL of pre-warmed growth medium is added to each well. AhR antagonist of interest are diluted at a 20×concentration in growth medium containing 2% DMSO, and 10 μL of compound solutions are added to respective wells in triplicate. After 1 hr, AHR activating ligand, such as TCDD, kynurenine, ITE (2-(1H-indole-3-ylcarbonyl)-4-thiazolecarboxylic methyl ester), VAF347, BNF (beta-naphthoflavone), FICZ (6-formylindolo(3,2-b) carbazole or other AHR ligands, is added with or without AHR antagonist for 24 hours, after which media will be removed and stored at −80 C for later cytokine analysis. At the end of the incubation, medium is aspirated off the CRC cells, and the cells washed with 100 μL of cold PBS solution. RNA is extracted via the TaqMan™ Gene Expression Cells-to-CT™ Kit (ThermoFisher) according to the manufacturer's protocol. The QuantStudio 6 Flex (Applied Biosciences) is used to analyze mRNA levels of CYP1A1 using GAPDH as the endogenous control. TaqMan™ probe sets for both genes are acquired from ThermoFisher. Samples are run in triplicate and data is analyzed using the QuantStudio software and reported as linear and log 2(ΔΔCT) values. Statistical analysis is performed using a two-tailed t-test comparing CYP1A1 levels in the presence of each individual compound to the vehicle negative control. Compounds with IC₅₀ in the range of the nanomolar concentration are considered for further evaluation. This assay can be used to confirm the inhibitory effect of the compounds prior to testing using an in vivo model.

### Example 29: Human PBMC (CD8+) Assay

Human donor blood (8 mL) is collected in sodium citrate CPT tubes and centrifuged at 1,600×g for 20 minutes at room temperature. Buffy coat containing PBMCs is collected and transferred to a 50 mL conical tube containing 30 mL of RPMI-1640 medium at room temperature (supplemented with penicillin-streptomycin). PBMCs samples are centrifuged at 400×g for 10 minutes at 10° C. The pelleted PBMCs are washed twice in 10 ml of RPMI-1640 medium (supplemented with penicillin-streptomycin), then resuspended in RPMI-1640 medium (supplemented with penicillin-streptomycin, fetal bovine serum, and L-Glutamine: RPMI-1640 complete medium). PBMCs are filtered through a 70-micron mesh to remove any cellular debris. The volume is adjusted to achieve 1.66×106 cells/mL, from which 180 μl (300,000 PBMCs) are added into each well in a 96-well plate (sterile, tissue culture treated, round bottom). PBMCs in a 96-well plate are rested for 30 minutes in a 37° C., 5% CO₂ incubator, then subsequently treated with 10 μl of indicated compound. For CD8+(Killing T cells) differentiation assay, PMBC are cultured (1-10×10⁴ cells) in RPMI-1640 complete medium for 2, 4 and 6 days and stimulated with 5 uL/ml ImmunoCult™ Human CD3/CD28/CD2 T Cell Activator; Stemcell #10990) with/without AhR antagonist Compounds. Cell viability was determined using a viability dye (eBioscience Fixable Viability Dye eFluor 780: ThermoFisher 65-0865-14) at 1:500 dilution. The cells were gated for CD8+, defined as Live, CD11c−, CD14−, CD19−, CD8+, CD4−, CD3+. Percent (%) CD8+ were calculated as percentage of CD8+ cells over total live T cells. Statistical analysis was performed with GraphPad Prism Software Using One-Way ANOVA.

### Example 30: Human PBMC Cytokine Assay

Human donor blood (8 mL) is collected in sodium citrate CPT tubes and centrifuged at 1,600×g for 20 minutes at room temperature. Buffy coat containing PBMCs is collected and transferred to a 50 mL conical tube containing 30 mL of RPMI-1640 medium at room temperature (supplemented with penicillin-streptomycin). PBMCs samples are centrifuged at 400×g for 10 minutes at 10° C. The pelleted PBMCs are washed twice in 10 ml of RPMI-1640 medium (supplemented with penicillin-streptomycin), then resuspended in RPMI-1640 medium (supplemented with penicillin-streptomycin, fetal bovine serum, and L-Glutamine: RPMI-1640 complete medium). PBMCs are filtered through a 70 micron mesh to remove any cellular debris. The volume is adjusted to achieve 1.66×106 cells/mL, from which 180 μl (300,000 PBMCs) are added into each well in a 96-well plate (sterile, tissue culture treated, round bottom). PBMCs in a 96-well plate are rested for 30 minutes in a 37° C., 5% CO₂ incubator, then subsequently treated with 10 μl of indicated compound. For cytokine secretion assay, PMBC are cultured (1-10×104 cells) in RPMI-1640 complete medium for 2, 4 and 6 days and stimulated with 5 uL/ml ImmunoCult™ Human CD3/CD28/CD2 T Cell Activator; Stemcell #10990) with/without AhR antagonist compounds. After 2, 4, and 6 days of incubation at 37° C., 5% CO2, 100 μL of cell supernatant is collected and transferred to a 96-well plate (non-tissue treated, flat bottom). The plate is centrifuged at 350×g for 5 minutes at room temperature, and then the clear supernatant transferred to a new 96-well plate (non-tissue treated, flat bottom). The remaining cells are tested for viability using CellTiter-Glo® Luminescent Cell Viability Assay (Promega). The supernatant is analyzed for IL22 and IFg), using Luminex Immunoassay Technology (MAGPIX System). Cytokine levels of PBMC treated DMSO control samples are set to 100%, and compound treated samples are expressed relative to this.

### Example 31: Solubility Determination Assay

The stock solutions of test compounds and control compound progesterone were prepared in DMSO at the concentrations of 10 mM. 15 μL of stock solution (10 mM) of each sample was placed in order into their proper 96-well rack. 485 μL of PBS pH 1.6 and pH 7.4 were added into each vial of the cap-less Solubility Sample plate. The assay was performed in singlet. One stir stick was added to each vial and then the vial was sealed using a molded PTFE/Silicone plug. The solubility sample plates were then transferred to the Eppendorf Thermomixer Comfort plate shaker and shaken at 25° C. at 1100 rpm for 2 hours. After completion of the 2 hours, plugs were removed and the stir sticks were removed using a big magnet. The samples from the Solubility Sample plate were transferred into the filter plate. Using the Vacuum Manifold, all the samples were filtered. An aliquot of 5 μL was taken from the filtrate followed by addition of 495 μL of a mixture of H₂O and acetonitrile containing internal standard (1:1). A certain proportion of ultrapure water was used to dilute the diluent according to the peak shape. The dilution factor was changed according to the solubility values and the LC-MS signal response.

From the 10 mM DMSO STD plate, 6 μL was transferred into the remaining empty plate, and then 194 μL of DMSO were added to that plate to have a STD concentration of 300 μM. From the 300 μM DMSO STD plate, 5 μL were transferred into the remaining empty plate, and then 495 μL of a mixture of H₂O and acetonitrile containing internal standard (1:1) were added to that plate to have a final STD concentration of 3 μM. A certain proportion of ultrapure water was used to dilute the diluent according to the peak shape. The concentrations of the standard samples were changed according to the LC-MS signal response.

The plate was placed into the well plate autosampler. The samples were evaluated by LC-MS/MS analysis.

All calculations were carried out using Microsoft Excel.

The filtrate was analyzed and quantified against a standard of known concentration using LC coupled with mass spectral peak identification and quantitation. Solubility values of the test compound and control compound were calculated as follows:

$$[ Sample ] = \frac{Area ratio Sample \times INJ VOL STD \times DF Sample \times [ STD \}}{Area ratio STD \times INJ VOL Sample}$$

Any value of the compounds that was not within the specified limits was rejected and the experiment was repeated.

The solubility of compounds of the disclosure in pH 1.6 and 7.4 buffers is reported in Table 11. “+++” indicates a solubility value equal to or greater than 1 “++” indicates a solubility value between 0.1 and 1 μM and “+” indicates a solubility value less than 0.1 μM.

TABLE 11

| | Aq. Solubility | Aq. Solubility |
|---|---|---|
| | at pH = 1.6 | at pH = 7.4 |
| Compound Name | (mM) | (mM) |
| Product 39 | +++ | +++ |
| Product 12 | ++ | ++ |
| Product 8 | ++ | + |
| Product 18 | +++ | +++ |
| Product 25 | +++ | +++ |
| Product 15 | ++ | +++ |
| Product 47 | +++ | ++ |
| Product 49 | +++ | +++ |

### Example 32: Hepatocyte Stability Assay

Preparation of working solutions: 10 mM stock solutions of test compound and positive control were prepared in DMSO. In separate conical tubes, the 10 mM solution of test compound and the positive control were diluted to 100 μM by combining 198 μL of 50% acetonitrile/50% water and 2 μL of 10 mM stock.

Preparation of Hepatocytes: Incubation medium (William's E Medium supplemented with GlutaMAX) and hepatocyte thawing medium were placed in a 37° C. water bath and allowed warming for at least 15 minutes prior to use. A vial of cryopreserved hepatocytes was transferred from storage, ensuring that vials remained at cryogenic temperatures until thawing process ensued. Cells were thawed by placing the vial in a 37° C. water bath and gently shaking the vials for 2 minutes. After thawing was completed, vial was sprayed with 70% ethanol and transferred to a biosafety cabinet. Wide-bore pipette tip were used to transfer hepatocytes into 50 mL conical tube containing thawing medium. The 50 mL conical tube were placed into a centrifuge and spun at 100 g for 10 minutes. Upon completion of spin, thawing medium was aspirated and resuspended hepatocytes in enough incubation medium to yield ˜1.5×10⁶ cells/mL. Using an AO/PI Staining, cells were counted and the viable cell density was determined. Cells with poor viability (<75% viability) were determined to be not acceptable for use. Cells were diluted with incubation medium to a working cell density of 0.5×10⁶ viable cells/mL.

Procedure for Stability Determination: 198 μL of hepatocytes were pipetted into each wells of a 96-well non-coated plate. The plate was placed in the incubator to allow the hepatocytes to warm for 10 minutes. 2 μL of the 100 μM test compound or positive control solutions were pipetted into respective wells of the 96-well non-coated plate to start the reaction. The plate was returned to the incubator for the designed time points. Well contents was transferred in 25 μL aliquots at time points of 0, 15, 30, 60, 90 and 120 minutes. The aliquots were then mixed with 6 volumes (150 μL) of acetonitrile containing internal standard, IS (100 nM alprazolam, 200 nM caffeine and 100 nM tolbutamide) to terminate the reaction. The mixture was vortex for 5 minutes. Samples were centrifuged for 45 minutes at 3,220 g. An aliquot of 100 μL of the supernatant was diluted by 100 μL ultra-pure water, and the mixture was used for LC/MS/MS analysis. All incubations were performed in duplicate.

Data Analysis: All calculations were carried out using Microsoft Excel. Peak areas were determined from extracted ion chromatograms. In vitro half-life (t_{1/2}) of parent compound was determined by regression analysis of the percent parent disappearance vs. time curve.

The in vitro half-life (in vitro t_{1/2}) was determined from the slope value:

in vitro t_{1/2=0.693}/k

Conversion of the in vitro t_{1/2} (in min) into the in vitro intrinsic clearance (in vitro CLᵢₙₜ, in μL/min/1×10⁶ cells) was done using the following equation (mean of duplicate determinations):
in vitro CLᵢₙₜ=kV/N
V=incubation volume (0.2 mL);
N=number of hepatocytes per well (0.1×10⁶ cells).

Data Processing Rules: The rules for data processing are shown in Table 12.

TABLE 12
Remaining
%

| | |
|---|---|
| ≥80% at | If T-test with p < 0.05 is obtained, report the calculated |
| 120 min | CLᵢₙₜ value; |
| | When the calculated CLint value <3.73, then report <3.73 |
| | instead of calculated value. If T-test with p < 0.05 is not |
| | obtained, then report <3.73 for CLᵢₙₜ value and >371.12 for |
| | t_{1/2} value when all the other data points fall in the range of |
| | 80%~120% (one data point within the range of 70%~130% is |
| | accepted, otherwise the experiment should be repeated). |
| <80% at | Always remove from the calculation all points with <10% left |
| 120 min | of 0.5 min sample, but leave at least 2 points |
| | If T-test with p < 0.05 is obtained, report the calculated |
| | CLᵢₙₜ value. |
| | If T-test with p < 0.05 is not obtained, the experiment must |
| | be repeated. |

The human, rat and mouse liver hepatocyte clearance of compounds of the disclosure is reported in Table 13. “+++” indicates a CLᵢₙₜ value less than 20 mL/min/Kg, “++” indicates a CLᵢₙₜ between 20 and 50 mL/min/Kg, and “+” indicates an CLᵢₙₜ above 50 mL/min/Kg.

TABLE 13

| | Human | Rat | Mouse |
|---|---|---|---|
| | hepatocyte | hepatocyte | hepatocyte |
| | clearance | clearance | clearance |
| Compound Name | (mL/min/Kg) | (mL/min/Kg) | (mL/min/Kg) |
| Product 39 | +++ | | |
| Product 12 | ++ | | |
| Product 8 | ++ | | + |
| Product 18 | ++ | | |
| Product 25 | +++ | + | + |
| Product 15 | +++ | + | |
| Product 47 | +++ | | |

### Example 33: Liver Microsome Stability Assay

The master solution was prepared according to Table 14.

TABLE 14

| | Stock | | | Final | |
|---|---|---|---|---|---|
| Reagent | Concentration | | Volume | Concentration | |
| Phosphate buffer | 100 | mM | 210 μL | 100 | mM |
| Microsomes | 20 | mg/mL | 6.25 μL | 0.5 | mg/mL |

Two separate experiments were performed as follows.

With Cofactors (NADPH): 25 μL of 10 mM NADPH was added to the incubations. The final concentrations of microsomes and NADPH were 0.5 mg/mL and 1 mM, respectively. The final concentration of microsomes was 0.5 mg/mL. The mixture was pre-warmed at 37° C. for 10 minutes. The reaction was started with the addition of 2.5 μL of 100 μM control compound or test compound solutions. Verapamil was used as positive control in this study. The final concentration of test compound or control compound was 1 μM. The incubation solution was incubated in water batch at 37° C. Aliquots of 25 μL were taken from the reaction solution at 0.5, 5, 15, 30 and 60 minutes. The reaction was stopped by the addition of 5 volumes of cold acetonitrile with IS (200 nM caffeine and 100 nM tolbutamide). Samples were centrifuged at 3, 220 g for 40 minutes. Aliquot of 100 μL of the supernatant was mixed with 100 μL of ultra-pure H₂O and then used for LC-MS/MS analysis.

Data Analysis: All calculations were carried out using Microsoft Excel. Peak areas were determined from extracted ion chromatograms. The slope value, k, was determined by linear regression of the natural logarithm of the remaining percentage of the parent drug vs. incubation time curve.

in vitro t_{1/2}=−(0.693/k)

The in vitro half-life (in vitro t_{1/2}) was determined from the slope value:

Conversion of the in vitro t_{1/2} (min) into the in vitro intrinsic clearance (in vitro CLᵢₙₜ, in μL/min/mg protein) was done using the following equation (mean of duplicate determinations):

$$in vitro {CL}_{int} = ( \frac{0.693}{( {t}_{1 / 2} )} ) * ( \frac{volume of incubation ( µL )}{amount of proteins ( mg )} )$$

The calculations of Scaled-up CLᵢₙₜ (mL/min/kg), Predicted CLH (mL/min/kg) and EH were done using the following equation:

Scaled-up CLᵢₙₜ=(0.693/t_{1/2})×(1/(microsomal protein concentration(0.5mg/mL)))×Scaling Factors;

Predicted CLH=(QH×Scaled-up CLᵢₙₜ×f_{ub})/(QH+Scaled-up CLᵢₙₜ×f_{ub});

EH=Predicted CLH/QH

where QH is the hepatic blood flow (mL/min/kg) (Table 14),
f_{ub} is the fraction of unbound drug in plasma which is assumed to be 1.

The scaling factors for intrinsic clearance prediction in the human and mouse microsomes are reported in Table 15.

TABLE 15

| | Microsomal | Liver weight | | |
|---|---|---|---|---|
| | protein per | per Kg of | Scaling | Hepatic |
| Species | gram of liver | body weight | factor* | blood flow |
| Human | 48.8 | 25.7 (human) | 1254.2 human) | 20.7 (human) |
| | | 40.0 (rat) | 1792 (rat) | 55.2 (rat) |

*Scaling Factor = (microsomal protein per gram of liver) × (liver weight per kilogram of body weight)

Data Processing Rules: The rules for data processing are shown in Table 16.

TABLE 16

| Remaining | |
|---|---|
| % | Processing Rules |
| ≥80% at | If T-test with p < 0.05 is obtained, report the calculated |
| 60 min | CLᵢₙₜ value |
| | If T-test with p < 0.05 is obtained, report the calculated |
| | CLᵢₙₜ value; |
| | When the calculated CLᵢₙₜ value <7.50, then report <7.50 |
| | instead of calculated value. |
| | If T-test with p < 0.05 is not obtained, then report <7.50 for |
| | CLᵢₙₜ value and >184.78 for t_{1/2} value when all the other data |
| | points fall in the range of 80%~120% (one data point within |
| | the range of 70%~130% is accepted, otherwise the experiment |
| | should be repeated). |
| <80% at | Always remove from the calculation all points with <10% left |
| 60 min | of 0.5 min sample, but leave at least 2 points |
| | If T-test with p < 0.05 is obtained, report the calculated CLᵢₙₜ |
| | value. |
| | If T-test with p < 0.05 is not obtained, the experiment must be |
| | repeated. |

### Example 34: Caco-2 Permeability Assay

Preparation of Caco-2 Cells: 50 μL and 25 mL of cell culture medium were added to each well of the Transwell insert and reservoir, respectively. The HTS transwell plates were incubated at 37° C., 5% CO₂ for 1 hour before cell seeding. Caco-2 cells were diluted to 6.86×10⁵ cells/mL with culture medium and 50 μL of cell suspension were dispensed into the filter well of the 96-well HTS Transwell plate. Cells were cultivated for 14-18 days in a cell culture incubator at 37° C., 5% CO₂, 95% relative humidity. Cell culture medium was replaced every other day, beginning no later than 24 hours after initial plating.

Assessment of Cell Monolayer Integrity: Medium was removed from the reservoir and each Transwell insert and replaced with prewarmed fresh culture medium. Transepithelial electrical resistance (TEER) across the monolayer was measured using Millicell Epithelial Volt-Ohm measuring system (Millipore, USA). The Plate was returned to the incubator once the measurement was done. The TEER value was calculated according to the following equation:

TEER measurement (ohms)×Area of membrane (cm²)=TEER value (ohm·cm²)

TEER value should be greater than 230 ohm·cm², which indicates the well-qualified Caco-2 monolayer.

Preparation of Solutions: 2 mM stock solutions in DMSO of control compounds were prepared and diluted with HBSS (10 mM HEPES, pH 7.4) to get 10 μM working solution. 0.2 mM stock solutions of test compounds in DMSO were prepared and diluted with HBSS (10 mM HEPES, pH 7.4 with 0.5% BSA) to get 1 μM working solution. Metoprolol, erythromycin and cimetidine were used as control compounds.

Performing the Drug Transport Assay: The Caco-2 plate was removed from the incubator. The monolayer was washed twice with pre-warmed HBSS (10 mM HEPES, pH 7.4). The plate was incubated at 37° C. for 30 minutes. To determine the rate of drug transport in the apical to basolateral direction, 125 μL of the working solution was added to the Transwell insert (apical compartment). A 50 μL sample was transferred immediately from the apical compartment to 200 μL of acetonitrile containing IS (100 nM alprazolam, 200 nM Caffeine and 100 nM tolbutamide) in a new 96-well plate as the initial donor sample (A-B) and it was vortexed at 1000 rpm for 10 minutes. The wells in the receiver plate (basolateral compartment) were filled with 235 μL of transport buffer. To determine the rate of drug transport in the basolateral to apical direction, 285 μL of the working solution were added to the receiver plate wells (basolateral compartment). A 50 μL sample was transferred immediately from the basolateral compartment to 200 μL of acetonitrile containing IS (100 nM alprazolam, 200 nM Caffeine and 100 nM tolbutamide) in a new 96-well plate as the initial donor sample (B-A) and it was vortexed at 1000 rpm for 10 minutes. The Transwell insert (apical compartment) was filled with 75 μL of transport buffer. The apical to basolateral direction and the basolateral to apical direction need to be done at the same time. The plates were incubated at 37° C. for 2 hours. At the end of the incubation, 50 μL samples from donor sides (apical compartment for Ap→B1 flux, and basolateral compartment for B1→Ap) and receiver sides (basolateral compartment for Ap→B1 flux, and apical compartment for B1→Ap) were transferred to wells of a new 96-well plate, followed by the addition of 4 volume of acetonitrile containing IS (100 nM alprazolam, 200 nM Caffeine and 100 nM tolbutamide). Samples were vortexed for 10 minutes, 50 μL samples were transferred to wells of a new 96-well plate, followed by the addition of 50 μL Hepes and 200 μL IS. All samples were vortexed for 10 minutes, and then centrifuged at 3,220 g for 40 minutes. An aliquot of 150 μL of the supernatant was mixed with an appropriate volume of ultra-pure water before LC-MS/MS analysis.

Data analysis: All calculations were carried out using Microsoft Excel. Peak areas were determined from extracted ion chromatograms. Lucifer yellow leakage of monolayer can be calculated using the following equation:

$$LY Leakage = ( \frac{{I}_{acceptor} \times 0.3}{{I}_{acceptor} \times 0.3 + {I}_{donor} \times 0.1} ) \times 100 \%$$

where I_{acceptor} is the fluorescence intensity in the acceptor well (0.3 mL), and
I_{donor} is the fluorescence intensity in the donor well (0.1 mL) and expressed as % leakage.

Lucifer yellow percentage amount transported values should be less than 1.5%. However, if the lucifer yellow percentage amount transported value for a particular transwell is higher than 1.5 but the determined digoxin Pₐₚₚ in that transwell is qualitatively similar to that determined in the replicate transwells then, based upon the scientific judgement of the responsible scientist, the monolayer is considered acceptable.

Apparent permeability (Papp) can be calculated for drug transport assays using the following equation:

where Pₐₚₚ is

$${P}_{app} = \frac{dQ / dt}{A \times {D}_{o}}$$

apparent permeability (cm/s×10⁻⁶);
dQ/dt is the rate of drug transport (pmol/second);
A is the surface area of the membrane (cm2);
Dₒ is the initial donor concentration (nM; pmol/cm₃).

Efflux ratio can be determined using the following equation:

$$Efflux Ratio = \frac{{P}_{app ( B - A )}}{{P}_{app ( A - B )}}$$

where P_{app(B-A)} indicates the apparent permeability coefficient in basolateral to apical direction,

and P_{app(A-B)} indicates the apparent permeability coefficient in apical to basolateral direction.

### Example 35: Plasma Protein Binding Determination with Ultracentrifugation Method

The frozen plasma (stored at −80° C.) was thawed in a 37° C. water bath, followed by centrifugation at 3,220 g for 10 minutes to remove clots. The supernatant was removed into a new tube as the spun plasma. The spun plasma was pre-warmed in a 37° C. water bath for 10 minutes. The stock solutions of test compounds were diluted to 200 μM in DMSO, and then spiked into the plasma. Duplicate samples were prepared. The final concentration of compound was 1.0 μM. The final concentration of organic solvent was 0.5%. Warfarin was used as positive control in the assay. 1.0 mL of the spiked plasma was transferred to a new balance ultracentrifuge tube. Samples were incubated at 37° C., 5% CO₂ for 30 minutes. After incubation, the balance ultracentrifuge tubes were centrifuged at 600,000 g for 5.5 hours at 37° C. After centrifugation, 50 μL solution was removed from the center of the ultracentrifuge tubes as the post-ultracentrifugation samples, followed by the addition of 50 μL blank plasma and 400 μL quench solution (acetonitrile containing internal standards (IS, 100 nM Alprazolam, 500 nM Labetalol and 2 μM Ketoprofen)) to precipitate protein and release compounds. Samples were vortexed for 2 minutes, followed by centrifugation at 20,000 g for 15 minutes at room temperature. The supernatant was diluted with ultrapure water and then used for LC-MS/MS analysis. Stability samples was prepared by transferring 50 μL of the spiked plasma to 0.6 mL tubes and incubated at 37° C., 5% CO₂ for 0.5 and 6 hours. After incubation, 50 μL PBS (100 mM, pH7.4) and 400 μL quench solution were added to the stability samples. And then stability samples were treated the same way as the post-ultracentrifugation samples. The supernatant was diluted with ultrapure water and then used for LC-MS/MS analysis. 0.5 hour time point samples were also used as no-spun controls. Time 0 samples were prepared by transferring 50 μL spiked plasma to 0.6 mL tubes containing 50 μL PBS, followed by the addition of 400 μL quench solution to precipitate protein and release compound. And then these samples were treated the same way as the post-ultracentrifugation samples. The supernatant was diluted with ultrapure water and then used for LC-MS/MS analysis.

Data Analysis: All calculations were carried out using Microsoft Excel. The concentrations of test compound in plasma samples and post-ultracentrifugation plasma was determined from peak areas. The percentages of test compound bound was calculated as follows:

% Unbound=(Peak Area post-ultracentrifugation/Peak Area non-spun control)×100%

% Bound=100%−% Unbound

Remaining % at 0.5 hr=Area ratio 0.5 hr/Area ratio 0 hr×100%

Remaining % at 6 hr=Area ratio 6 hr/Area ratio 0 hr×100%

$$LogK = Log ( \frac{\% Bound}{100 - \% Bound} )$$

### Example 36: CYP Inhibition Assay

Stock solutions of test compounds were prepared in DMSO at the concentrations of 10 mM. Stock solution was diluted to 2 mM with acetonitrile. The final concentration of test compounds was 10 μM. The concentration of positive inhibitor is listed in Table 17. For the stock solution preparation, if the positive control could not be well dissolved in the mixture of DMSO and acetonitrile (1:4) at the highest concentration, another mixture of acetonitrile and DMSO, the mixture of acetonitrile and H₂O or DMSO will be used to dissolve the compound.

TABLE 17
Table 4. Positive inhibitor nominal concentration

| | | Conc. of | Final |
|---|---|---|---|
| | | stock | conc. in |
| CYP Isoform | Positive control | solution (μM) | system (μM) |
| CYP2D6, 3A4 | Quinidine, Ketoconazole | 100 μM | 0.5 μM |

Preparation details of these substrates are given in Table 18. The substrate solutions are stored in a −20° C. freezer and warmed to room temperature prior to use.

TABLE 18
Table 5. Preparation of Substrate Stock Solution

| | | Conc. of | Final | Incu- |
|---|---|---|---|---|
| CYP | | stock | conc. in | bation |
| Isoform | Substrate | solution (mM) | system (μM) | Time |
| 2D6 | Dextromethorphan | 0.4 (in ACN) | 2 | 20 min |
| 3A4 | Midazolam | 0.2 (in MeOH + | 1 | 5 min |
| | | ACN) | | |

Preparation of Phosphate Buffer (100 mmol/L, pH 7.4): To prepare the Solution A, 7.098 g of disodium hydrogen phosphate were weighed out and added into 500 mL of pure water, then sonicated to dissolve the content. To prepare the Solution B, 3.400 g of potassium dihydrogen phosphate were weighed out and added into 250 mL of pure water, then sonicated to dissolve the content. Solution A was placed on a stirrer and slowly Solution B was added into Solution A until the pH reached 7.4. Preparation of 10 mmol/L NADPH Solution: NADPH was dissolved at 8.334 mg/mL in phosphate buffer; the solution was freshly prepared prior to use.

The master solution was prepared according to Table 19. The incubation was carried out in 96 deep well plates. The following volumes were dispensed into each well of the incubation plate: 179 μL of the substrate and HLM mixture in phosphate buffer, 1 μL of the compound working solution, or vehicle (mixture of DMSO and acetonitrile (1:4)). The incubation plate was placed into the water bath and pre-warmed at 37° C. for 15 minutes before the reactions was started by the addition of 20 μL of 10 mmol/L NADPH solution in phosphate buffer. After the addition of NADPH, the incubation plate was incubated at 37° C. for corresponding time. The assay was performed in duplicate.

TABLE 19
Table 6. Preparation of master solution

| Buffer | Stock Concentration | | Volume | Final Concentration | |
|---|---|---|---|---|---|
| Microsomes | 20 | mg/mL | 2 μL | 0.2 | mg/mL |
| Phosphate buffer | 100 | mM | 176 μL | 100 | mM |

| | | | |
|---|---|---|---|
| Substrate | — | 1 μL | — |

The reaction was quenched by the addition of 1.5 volume (300 μL) of cold acetonitrile containing 3% formic acid and internal standards (200 nM Labetalol, 200 nM Alprazolam and 200 nM tolbutamide). The plate was centrifuged at 3,220 g for 40 minutes. 100 μL of the supernatant was transferred to a new plate. The supernatant was diluted with 100 μL pure water. The samples were mixed well and analyzed using UPLC/MS/MS.

Data Analysis: The automatic peak integration areas are checked for all the samples. The Analyte Peak Area and Internal Standard Peak Area are exported into excel spreadsheet. The inhibition of each P450 enzyme in human liver microsomes is measured as the percentage decrease in the activity of marker metabolite formation compared to non-inhibited controls (=100% activity).

The percentage of remaining activity was calculated as follows:

Area Ratio=Peak Area Analyte/Peak Area Internal Standard

Remaining Activity (%)=Area Ratio test compound/Area Ratio vehicle*100%

Inhibition %=100−Remaining Activity (%)

### Example 37: hERG Inhibition Assay

hERG stably expressed HEK 293 cell line (Cat #K1236) was purchased from Invitrogen. The cells are cultured in 85% DMEM, 10% dialyzed FBS, 0.1 mM NEAA, 25 mM HEPES, 100 U/mL Penicillin-Streptomycin and 5 μg/mL Blasticidin and 400 m/mL Geneticin. Cells are split using TrypLE™ Express about three times a week and maintained between −40% to −80% confluence. Before the assay, the cells were onto the coverslips at 5×105 cells/per 6 cm cell culture dish and induced with doxycycline at 1 μg/mL for 48 hours.

External solution (in mM): 132 NaCl, 4 KCl, 3 CaCl₂, 0.5 MgCl₂, 11.1 glucose, and 10 HEPES (pH adjusted to 7.35 with NaOH). Internal solution (in mM): 140 KCl, 2 MgCl₂, 10 EGTA, 10 HEPES and 5 MgATP (pH adjusted to 7.35 with KOH). Working solution preparation for test compound: test compounds were initially prepared in DMSO with final concentration of 10 mM as stock solution. Stock solution of each compound was serial-diluted by ratio of 1:3 with DMSO to prepare additional 3 intermediate solutions including 3.33, 1.11 and 0.37 mM.

Before performing the hERG assay, the working solutions were prepared by dilution of 10, 3.33, 1.11, and 0.37 mM intermediate solutions in 1000 folds using extracellular solution, while 30 μM working solution was prepared by 333.333-folds dilution of 10 mM DMSO stock. so that the final concentration of working solution was 30, 10, 3.33, 1.11 and 0.37 μM. The final DMSO concentration in working solutions was maintained in range of 0.1-0.3% (v/v).

Experimental procedure: the coverslip was removed from the cell culture dish and placed it on the microscope stage in bath chamber. A desirable cell was located using the ×10 objective. The tip of the electrode was located under the microscope using the ×10 objective by focusing above the plane of the cells. Once the tip was in focus, the electrode was advanced downwards towards the cell using the coarse controls of the manipulator, while simultaneously moving the objective to keep the tip in focus. When directly over the cell, the fine controls of the manipulator were used to approach the surface of the cell in small steps, by using the ×40 objective. Gentle suction was applied through the side-port of the electrode holder to form a gigaohm seal.

Cfast was used to remove the capacity current that is in coincidence with the voltage step. The whole cell configuration was obtained by applying repetitive, brief, strong suction until the membrane patch has ruptured. membrane potential was set to −60 mV at this point to ensure that hERG channels were not open. The spikes of capacity current was then cancelled using the Cslow on the amplifier.

Holding potential was set to −90 mV for 500 ms; current was recorder at 20 kHz and filtered at 10 kHz. Leaking current was tested at −80 mV for 500 ms.

The hERG current was elicited by depolarizing at +30 mV for 4.8 seconds and then the voltage was taken back to −50 mV for 5.2 seconds to remove the inactivation and observe the deactivating tail current. The maximum amount of tail current size was used to determine hERG current amplitude. Current was recorded for 120 seconds to assess current stability. Only stable cells with recording parameters above threshold were proceeded with further drug administrations. Vehicle control was applied to the cells to establish the baseline. Once the hERG current was found to be stabilized for 5 minutes, working solution was applied. hERG current in the presence of test compound were recorded for approximately 5 minutes to reach steady state and then 5 sweeps were captured. For dose response testing, 5 doses of test compound was applied to the cells cumulatively from low to high concentrations. In order to ensure the good performance of cultured cells and operations, the positive control, Dofetilide, with 5 doses was also used to test the same batch of cells.

The following criteria were used to determine data acceptability: initial seal resistance >1 GΩ; leak currents <50% of the control peak tail currents at any time; the peak tail amplitude >300 pA; membrane resistance Rm >500 MΩ; access resistance (Ra)<15 MΩ; apparent run-down of peak current <2.5% per min.

Data that met the above criteria for hERG current quality were further analyzed as the following steps. Percent current inhibition was calculated using the following equation: (Note: PatchMaster or Clampfit software were used to extract the peak current from the original data).

$$Peak current inhibition = ( 1 - \frac{Peak tail {current}_{compound}}{Peak tail {current}_{blank vehicle}} ) \times 100$$

The dose response curve of test compounds was plotted with % inhibition against the concentration of test compounds using Graphpad Prism 6.0, and fit the data to a sigmoid dose-response curve with a variable slope.

### Example 38: In Vivo Rat PK Studies

The studies were conducted in male SD rats, three rats per group. Compounds were dosed 1.0 mg/Kg i.v. (vehicle ethanol: % PEG400 in deionized water, in proportions suitable for dosing a clear solution) and 3.0 mg/Kg p.o. (vehicle: 1% methyl cellulose: 1,500 cP in DI water (w/v)).

### In Vivo Model

Balb/c and C57BL/6 mice will be purchased from certified vendors and used in the studies. Animal husbandry, feeding and health conditions will be according to animal welfare guidelines. AHR agonist, and test compounds will be formulated in suitable vehicles.

CYP1A1 Levels in liver and spleen: C57BL/6 mice (n=3 per group) will be treated with AHR agonist alone or with AHR antagonist. Animals will be sacrificed at 4 or 10 hours after treatment upon which their livers and spleens will be collected and subsequent RT-PCR will be performed to determine levels of Cyplal and GAPDH. Data analysis will be performed including normalization to GAPDH housekeeping gene and to control treatment.

### Efficacy Study AHR Antagonist and Checkpoint Inhibitor Anti-PD-1 in the Mouse Colorectal Cancer Model CT26 in Balb/c Mice

CT26 is a murine colon carcinoma cell line obtained from ATCC. CT26 cells will be cultured in RPMI supplemented with 10% FBS.1 106 CT26 cells in 100 μL PBS will be implanted subcutaneously in 6-8-week-old Balb/c mice. Dosing for the efficacy study will start 5 days after implantation and after the tumor have reached 100 mm3: AHR antagonist will be dosed orally, every day (QD) at 30 mg/kg and 10 mg/kg for 3 weeks. anti-PD-1 (BioXcell RMP1-14) will be twice a week, intraperitoneally at 10 mg/kg for five total doses. Tumors will be monitored by caliper measurement every day and body weight will be measured three times per week. At the end point, tumors will be recovered and analyzed by Flowcytometry and or IHC for infiltrated tumor immune cells.

### Efficacy Study AHR Antagonist and Checkpoint Inhibitor Anti-PD-1 in the Mouse Colorectal Cancer Model MC38 in C57BL/6

MC38 is a murine colon carcinoma cell line obtained from Kerafast. MC38 cells will be cultured in RPMI supplemented with 10% FBS. 1 106 MC38 cells in 100 μL PBS will be implanted subcutaneously in 6-8-week-old C57BL/6 mice. Dosing for the efficacy study will start 5 days after implantation and after the tumor have reached 100 mm3: AHR antagonist will be dosed orally, every day (QD) at 30 mg/kg and 10 mg/kg for 3 weeks. anti-PD-1 (BioXcell RMP1-14) will be twice a week, intraperitoneally at 10 mg/kg for five total doses. Tumors will be monitored by caliper measurement every day and body weight will be measured three times per week. At the end point, tumors will be recovered and analyzed by Flowcytometry and or IHC for infiltrated tumor immune cells

### AHR-Dependent Gene Expression in Tumor, Spleen and Liver:

AHR-dependent gene expression will be measured in tissue samples such as tumor or liver. RNA will be extracted from the tissue via RNA isolation kit such as Qiagen. The RNA extraction will be done from total cells or cells post-sorting for specific populations of cells such as tumor cells, tumor associated-T cells, tumor associated-myeloid cells, Tumor associate-macrophages or others. Gene expression will be determined by quantitative RT-PCR using probes for specific genes including a housekeeping gene such as Gapdh for normalization. AHR-dependent genes will be examined include but are not limited to: CYP1A1, CYP1B1, AHRR, IDO1, IDO2, IL22, IL6, VEGFA, STAT3, cdc2, MMP13, MMP-9.

1. A compound chosen from 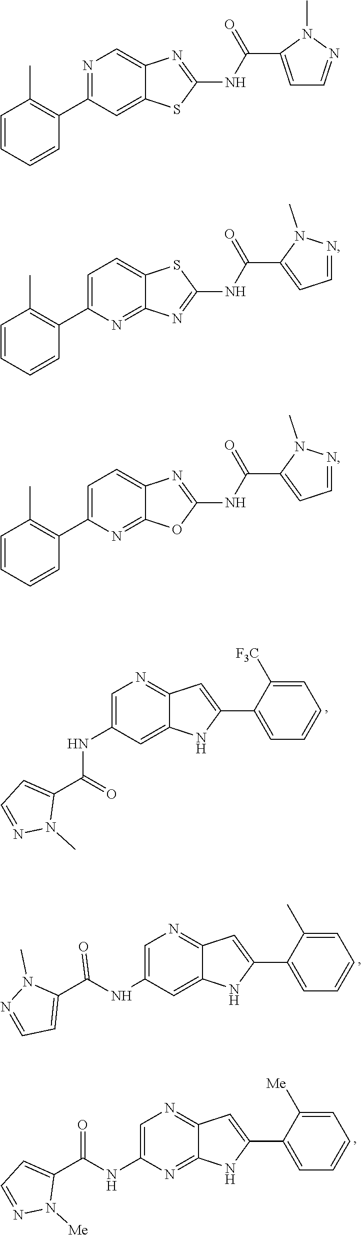 [SMILES: Cc1ccccc1-c1cc2sc(NC(=O)c3ccnn3C)nc2cn1 ; Cc1ccccc1-c1ccc2sc(NC(=O)c3ccnn3C)nc2n1 ; Cc1ccccc1-c1ccc2nc(NC(=O)c3ccnn3C)oc2n1 ; Cn1nccc1C(=O)Nc1cnc2cc(-c3ccccc3C(F)(F)F)[nH]c2c1 ; Cc1ccccc1-c1cc2ncc(NC(=O)c3ccnn3C)cc2[nH]1 ; Cc1ccccc1-c1cc2ncc(NC(=O)c3ccnn3C)nc2[nH]1]
and pharmaceutically acceptable salts of any of the foregoing.

2. A compound chosen from 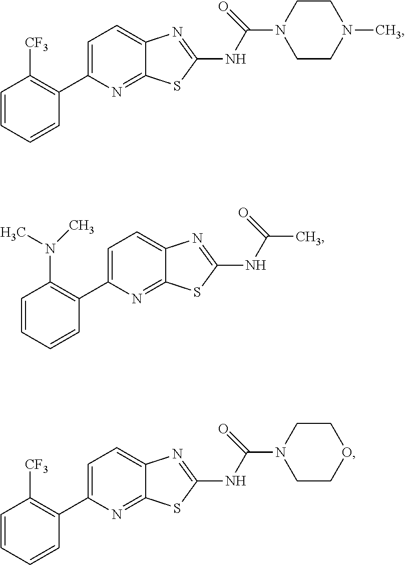 [SMILES: CN1CCN(C(=O)Nc2nc3ccc(-c4ccccc4C(F)(F)F)nc3s2)CC1 ; CC(=O)Nc1nc2ccc(-c3ccccc3N(C)C)nc2s1 ; O=C(Nc1nc2ccc(-c3ccccc3C(F)(F)F)nc2s1)N1CCOCC1]
and pharmaceutically acceptable salts of any of the foregoing.

3. A pharmaceutical composition comprising at least one entity chosen from compounds of claim 1, the compounds of claim 2
compounds of formula I 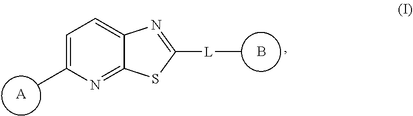
compounds of formula (II) 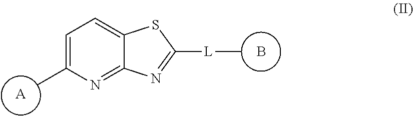
with the proviso that the compound is not 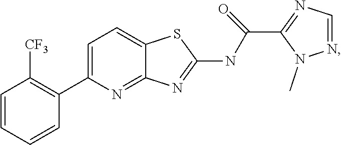 [SMILES: Cn1ncnc1C(=O)Nc1nc2nc(-c3ccccc3C(F)(F)F)ccc2s1]
compounds of formula (III) 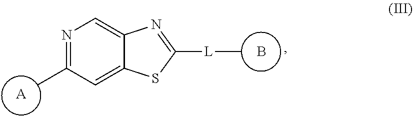
compounds of formula (IV) 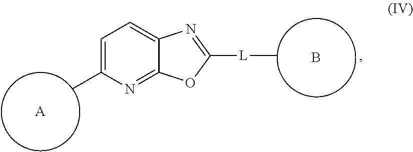
compounds of formula (V) 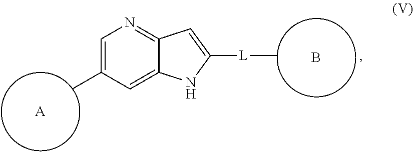
compounds of formula (VI) 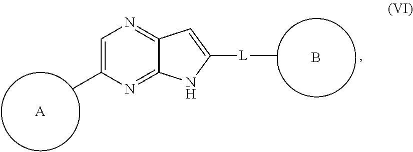
compounds of formula (VII) 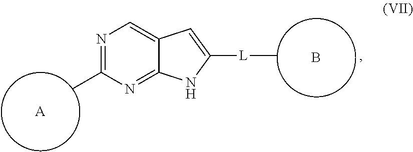
and pharmaceutically acceptable salts of any of the foregoing, and at least one pharmaceutically acceptable excipient.

4. A method of treating a disease or condition associated with aberrant AhR signaling in a subject in need thereof comprising administering to the subject a pharmaceutical composition according to claim 3 or a therapeutically effective amount of at least one entity chosen from
compounds of claim 1,
compounds of claim 2,
compounds of formula I 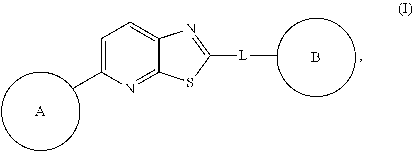
compounds of formula (II) 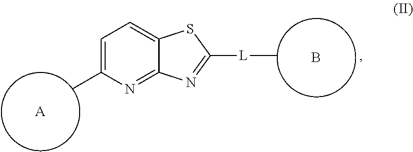
with the proviso that the compound is not 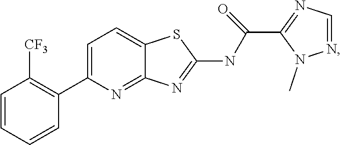 [SMILES: Cn1ncnc1C(=O)Nc1nc2nc(-c3ccccc3C(F)(F)F)ccc2s1]
compounds of formula (III) 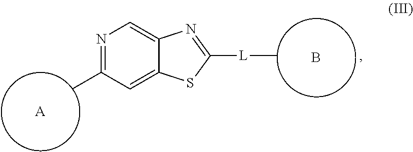
compounds of formula (IV) 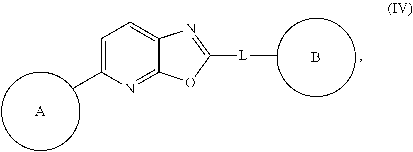
compounds of formula (V) 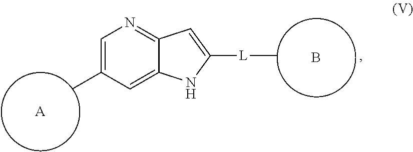
compounds of formula (VI) 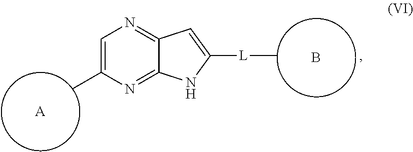
compounds of formula (VII) 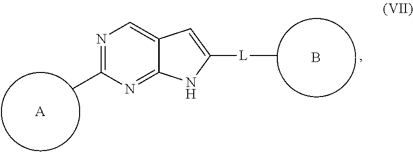
and pharmaceutically acceptable salts of any of the foregoing,
wherein
Ring A is chosen from optionally substituted aryls, optionally substituted heteroaryls, optionally substituted cycloalkyls, and optionally substituted heterocycloalkyls;
Ring B is chosen from optionally substituted aryls, optionally substituted heteroaryls, optionally substituted cycloalkyls, and optionally substituted heterocycloalkyls; and
L is chosen from a bond and —NT¹-C(O)—, wherein T¹ is H or Me.

5. A method of treating a disease or condition associated with aberrant AhR signaling in a subject in need thereof comprising administering to the subject a therapeutically effective amount of a pharmaceutical composition according to claim 3 or a therapeutically effective amount of at least one entity chosen from compounds of any one of claims 1 and 2, compounds of Formula I 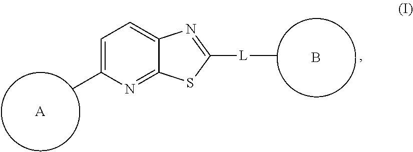
compounds of formula (II) 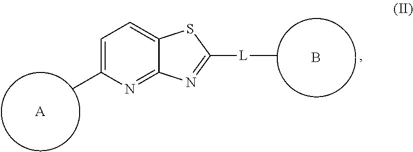
with the proviso that the compound is not 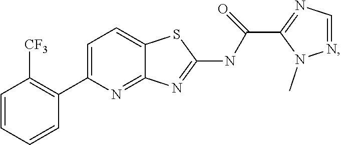 [SMILES: Cn1ncnc1C(=O)Nc1nc2nc(-c3ccccc3C(F)(F)F)ccc2s1]
compounds of formula (III) 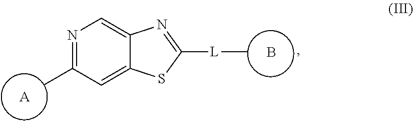
compounds of formula (IV) 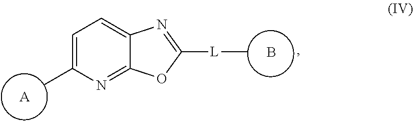
compounds of formula (V) 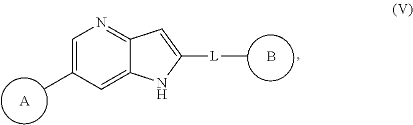
compounds of formula (VI) 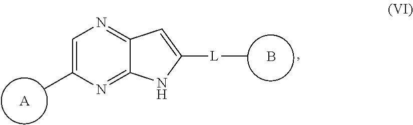
compounds of formula (VII) 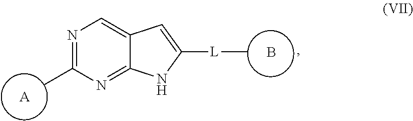
and pharmaceutically acceptable salts thereof,
wherein
Ring A is chosen from optionally substituted aryls, optionally substituted heteroaryls, optionally substituted cycloalkyls, and optionally substituted heterocycloalkyls;
Ring B is chosen from optionally substituted aryls, optionally substituted heteroaryls, optionally substituted cycloalkyls, and optionally substituted heterocycloalkyls; and
L is chosen from a bond and —NT¹-C(O)—, wherein T¹ is H or Me.

6. A method of treating a disease or condition mediated by AhR signaling in a subject in need thereof comprising administering to the subject a therapeutically effective amount of a pharmaceutical composition according to claim 3 or a therapeutically effective amount of at least one entity chosen from compounds of any one of claims 1 and 2, compounds of Formula I 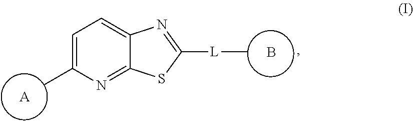
compounds of formula (II) 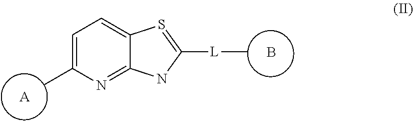
with the proviso that the compound is not 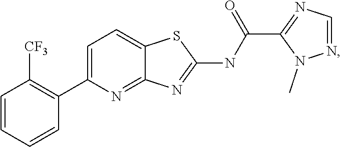 [SMILES: Cn1ncnc1C(=O)Nc1nc2nc(-c3ccccc3C(F)(F)F)ccc2s1]
compounds of formula (III) 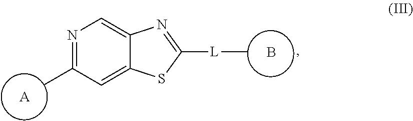
compounds of formula (IV) 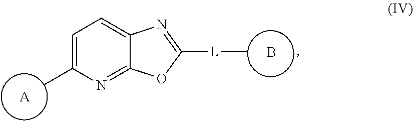
compounds of formula (V) 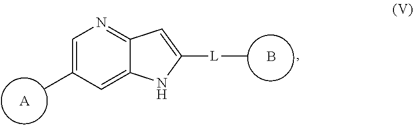
compounds of formula (VI) 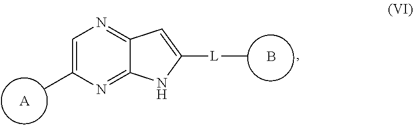
compounds of formula (VII) 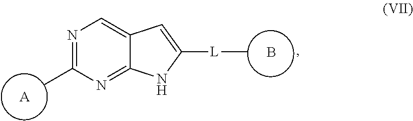
and pharmaceutically acceptable salts thereof,
wherein
Ring A is chosen from optionally substituted aryls, optionally substituted heteroaryls, optionally substituted cycloalkyls, and optionally substituted heterocycloalkyls;
Ring B is chosen from optionally substituted aryls, optionally substituted heteroaryls, optionally substituted cycloalkyls, and optionally substituted heterocycloalkyls; and
L is chosen from a bond and —NT¹-C(O)—, wherein T¹ is H or Me.

7. A method of inhibiting cancer cell proliferation mediated by AhR signaling in a subject in need thereof comprising administering to the subject a pharmaceutical composition according to claim 3 or a therapeutically effective amount of at least one entity chosen from compounds of any one of claims 1 and 2, compounds of Formula I 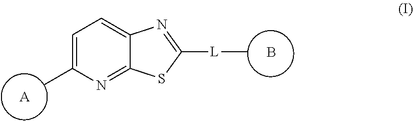
compounds of formula (II) 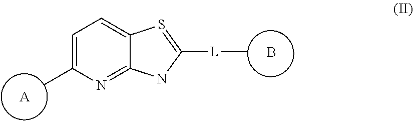
with the proviso that the compound is not 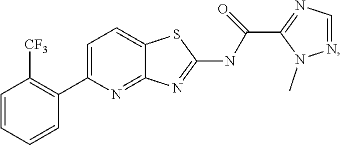 [SMILES: Cn1ncnc1C(=O)Nc1nc2nc(-c3ccccc3C(F)(F)F)ccc2s1]
compounds of formula (III) 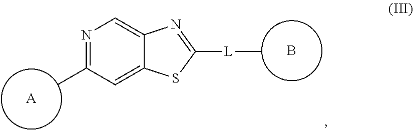
compounds of formula (IV) 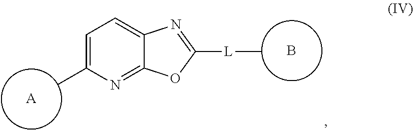
compounds of formula (V) 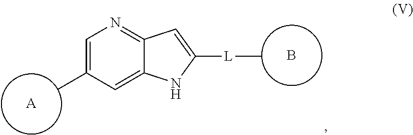
compounds of formula (VI) 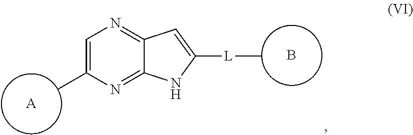
compounds of formula (VII) 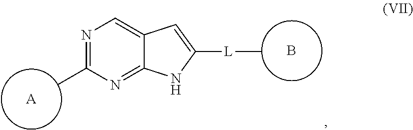
and pharmaceutically acceptable salts thereof,
wherein
Ring A is chosen from optionally substituted aryls, optionally substituted heteroaryls, optionally substituted cycloalkyls, and optionally substituted heterocycloalkyls;
Ring B is chosen from optionally substituted aryls, optionally substituted heteroaryls, optionally substituted cycloalkyls, and optionally substituted heterocycloalkyls; and
L is chosen from a bond and —NT¹-C(O)—, wherein T¹ is H or Me.

8. A method of inhibiting tumor cell invasion or metastasis mediated by AhR signaling in a subject in need thereof comprising administering to the subject a therapeutically effective amount of a pharmaceutical composition according to claim 3 or a therapeutically effective amount of at least one entity chosen from compounds of any one of claims 1 and 2, compounds of Formula I 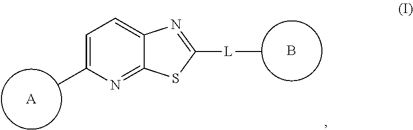
compounds of formula (II) 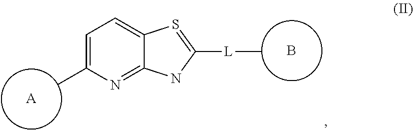
with the proviso that the compound is not 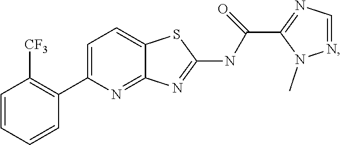 [SMILES: Cn1ncnc1C(=O)Nc1nc2nc(-c3ccccc3C(F)(F)F)ccc2s1]
compounds of formula (III) 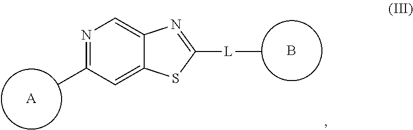
compounds of formula (IV) 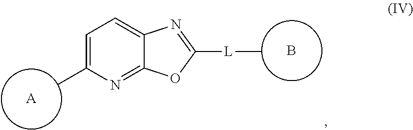
compounds of formula (V) 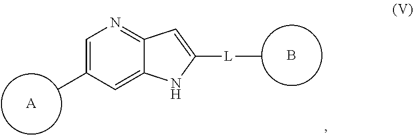
compounds of formula (VI) 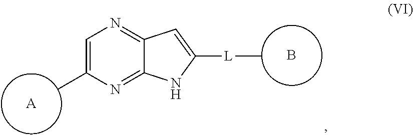
compounds of formula (VII) 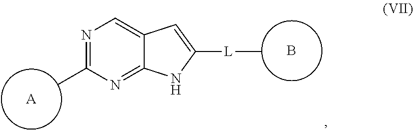
and pharmaceutically acceptable salts thereof,
wherein
Ring A is chosen from optionally substituted aryls, optionally substituted heteroaryls, optionally substituted cycloalkyls, and optionally substituted heterocycloalkyls;
Ring B is chosen from optionally substituted aryls, optionally substituted heteroaryls, optionally substituted cycloalkyls, and optionally substituted heterocycloalkyls; and
L is chosen from a bond and —NT¹-C(O)—, wherein T¹ is H or Me.

9. The method according to any one of claims 4-8, wherein the at least one entity is chosen from compounds of any one of claims 1 and 2 and pharmaceutically acceptable salts thereof.

10. The method according to any one of claims 4-8, wherein the least one entity is chosen from:
(i) 1-Methyl-N-(5-(2-(trifluoromethyl)phenyl)thiazolo[5,4-b]pyridin-2-yl)-1H-pyrazole-5-carboxamide;
(ii) 1-Methyl-N-(5-(2-(trifluoromethyl)phenyl)thiazolo[5,4-b]pyridin-2-yl)-1H-1,2,4-triazole-5-carboxamide;
(iii) 1-Methyl-N-(5-(o-tolyl)thiazolo[5,4-b]pyridin-2-yl)-1H-1,2,4-triazole-5-carboxamide;
(iv) N-(5-(2-(difluoromethyl)phenyl)thiazolo[5,4-b]pyridin-2-yl)-1-methyl-1H-pyrazole-5-carboxamide;
(v) N-(5-(2-(difluoromethyl)phenyl)thiazolo[5,4-b]pyridin-2-yl)-1-methyl-1H-1,2,4-triazole-5-carboxamide;
(vi) N-(5-(o-tolyl)thiazolo[5,4-b]pyridin-2-yl)picolinamide;
(vii) N-(5-(o-tolyl)thiazolo[5,4-b]pyridin-2-yl)nicotinamide;
(viii) N-(5-(o-tolyl)thiazolo[5,4-b]pyridin-2-yl)isonicotinamide;
(ix) N-(5-(2-methoxyphenyl)thiazolo[5,4-b]pyridin-2-yl)-1-methyl-1H-1,2,4-triazole-5-carboxamide;
(x) N-(5-(2-(difluoromethyl)-5-fluorophenyl)thiazolo[5,4-b]pyridin-2-yl)-1-methyl-1H-pyrazole-5-carboxamide;
(xi) N-(5-(2-(difluoromethyl)-5-fluorophenyl)thiazolo[5,4-b]pyridin-2-yl)-1-methyl-1H-1,2,4-triazole-5-carboxamide;
(xii) N-(5-(2-methoxyphenyl)thiazolo[5,4-b]pyridin-2-yl)picolinamide;
(xiii) N-(5-(2-methoxyphenyl)thiazolo[5,4-b]pyridin-2-yl)nicotinamide;
(xiv) N-(5-(2-methoxyphenyl)thiazolo[5,4-b]pyridin-2-yl)isonicotinamide;
(xv) 1-Methyl-N-(5-(o-tolyl)thiazolo[5,4-b]pyridin-2-yl)-1H-pyrazole-5-carboxamide;
(xvi) N-(5-(3-methoxyphenyl)thiazolo[5,4-b]pyridin-2-yl)-1-methyl-1H-pyrazole-5-carboxamide;
(xvii) N-(5-(3-(dimethylcarbamoyl)phenyl)thiazolo[5,4-b]pyridin-2-yl)-1-methyl-1H-pyrazole-5-carboxamide;
(xviii) N-(5-(2-(dimethylamino)phenyl)thiazolo[5,4-b]pyridin-2-yl)-1-methyl-1H-pyrazole-5-carboxamide;
(xix) N-(5-(3-(dimethylamino)phenyl)thiazolo[5,4-b]pyridin-2-yl)-1-methyl-1H-pyrazole-5-carboxamide;
(xx) N-(5-(4-(dimethylamino)phenyl)thiazolo[5,4-b]pyridin-2-yl)-1-methyl-1H-pyrazole-5-carboxamide;
(xxi) 1-Methyl-N-(5-(2-(pyrrolidin-1-yl)phenyl)thiazolo[5,4-b]pyridin-2-yl)-1H-pyrazole-5-carboxamide;
(xxii) 1-Methyl-N-(5-(1-methyl-1H-pyrazol-4-yl)thiazolo[5,4-b]pyridin-2-yl)-1H-pyrazole-5-carboxamide;
(xxiii) N-(5-(2-(dimethylamino)phenyl)thiazolo[5,4-b]pyridin-2-yl)acetamide;
(xxiv) N-(5-(2-hydroxyphenyl)thiazolo[5,4-b]pyridin-2-yl)-1-methyl-1H-pyrazole-5-carboxamide;
(xxv) N-(5-(2-methoxyphenyl)thiazolo[5,4-b]pyridin-2-yl)-1-methyl-1H-pyrazole-5-carboxamide;
(xxvi) 1-Methyl-N-(5-(pyridin-3-yl)thiazolo[5,4-b]pyridin-2-yl)-1H-pyrazole-5-carboxamide;
(xxvii) 1-Methyl-N-(5-(pyridin-2-yl)thiazolo[5,4-b]pyridin-2-yl)-1H-pyrazole-5-carboxamide;
(xxviii) 1-Methyl-N-(5-(thiazol-2-yl)thiazolo[5,4-b]pyridin-2-yl)-1H-pyrazole-5-carboxamide;
(xxix) N-(5-(4,5-dimethylthiazol-2-yl)thiazolo[5,4-b]pyridin-2-yl)-1-methyl-1H-pyrazole-5-carboxamide;
(xxx) 1-Methyl-N-(5-(5-methylthiazol-2-yl)thiazolo[5,4-b]pyridin-2-yl)-1H-pyrazole-5-carboxamide;
(xxxi) 1-Methyl-N-(5-(4-methylthiazol-5-yl)thiazolo[5,4-b]pyridin-2-yl)-1H-pyrazole-5-carboxamide;
(xxxii) 1-Methyl-N-(5-(4-methylthiazol-2-yl)thiazolo[5,4-b]pyridin-2-yl)-1H-pyrazole-5-carboxamide;
(xxxiii) N-(5-(2,4-dimethylthiazol-5-yl)thiazolo[5,4-b]pyridin-2-yl)-1-methyl-1H-pyrazole-5-carboxamide;
(xxxiv) N-(5-(2-isobutyramidophenyl)thiazolo[5,4-b]pyridin-2-yl)-1-methyl-1H-pyrazole-5-carboxamide;
(xxxv) N-(5-(2-acetamidophenyl)thiazolo[5,4-b]pyridin-2-yl)-1-methyl-1H-pyrazole-5-carboxamide;
(xxxvi) 1-Methyl-N-(5-(2-(N-methylacetamido)phenyl)thiazolo[5,4-b]pyridin-2-yl)-1H-pyrazole-5-carboxamide;
(xxxvii) N-(5-(2-((2-hydroxyethyl)(methyl)amino)phenyl)thiazolo[5,4-b]pyridin-2-yl)-1-methyl-1H-pyrazole-5-carboxamide;
(xxxviii) N-Methyl-N-(5-(o-tolyl)thiazolo[5,4-b]pyridin-2-yl)picolinamide;
(xxxix) N-(5-(o-tolyl)thiazolo [5,4-b]pyridin-2-yl)morpholine-4-carboxamide;
(xl) 4-Methyl-N-(5-(o-tolyl)thiazolo[5,4-b]pyridin-2-yl)piperazine-1-carboxamide; and
pharmaceutically acceptable salts of any of the foregoing.

11. The method according to any one of claims 4-8, wherein Ring A is chosen from optionally substituted 6-10 membered aryls, optionally substituted 5-10 membered heteroaryls, optionally substituted 3-10 membered cycloalkyls, and optionally substituted 3-10 membered heterocycloalkyls.

12. The method according to any one of claims 4-11, wherein Ring A is chosen from phenyl, pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, and pyrimidinyl, each of which may be substituted with one or more substituents, which may be the same or different.

13. The method according to any one of claims 4-11, wherein Ring A is chosen from

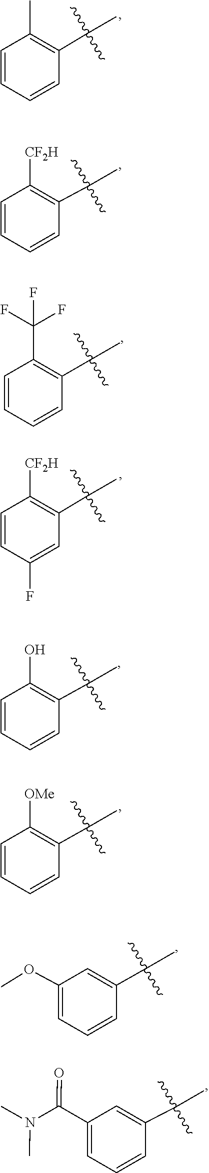

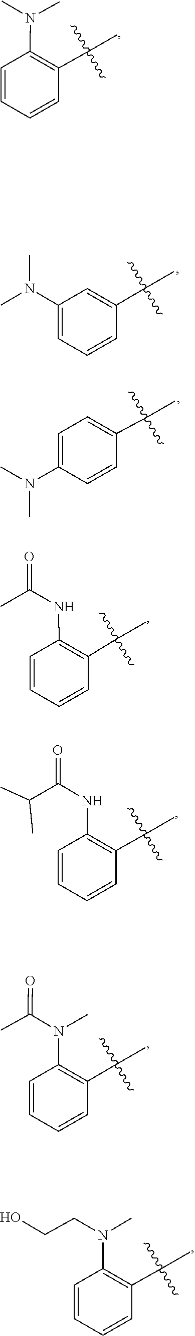

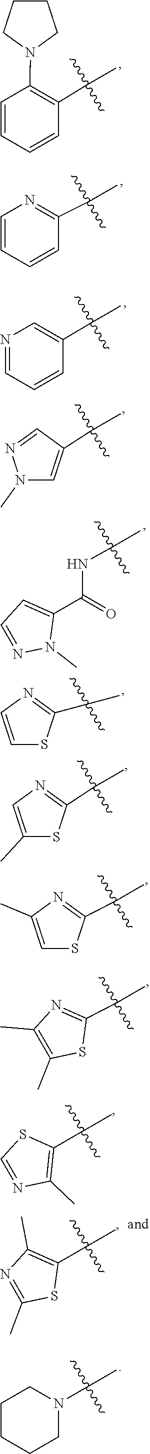

14. The method according to any one of claims 4-13, wherein Ring B is chosen from optionally substituted 6-10 membered aryls, optionally substituted 5-10 membered heteroaryls, optionally substituted 3-10 membered cycloalkyls, and optionally substituted 3-10 membered heterocycloalkyls.

15. The method according to any one of claims 4-14, wherein Ring B is chosen from phenyl, pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, pyridinonyl, pyrimidinyl, piperidinyl, piperazinyl, and morpholinyl each of which may be substituted with one or more substituents, which may be the same or different.

16. The method according to any one of claims 4-14, wherein Ring B is chosen from

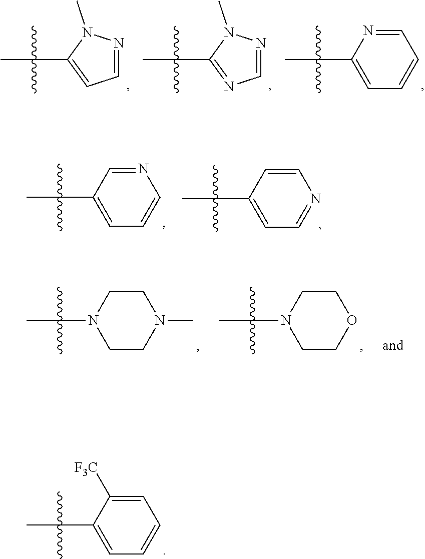

17. The method according to any one of claims 4-16, wherein L is a bond, —NH(C═O)—, or —NCH₃(C═O)—.

18. The method of any one of claims 4-17, wherein the disease is chosen from cancers.

19. The method of any one of claims 4-18, wherein the disease is chosen from breast cancers, respiratory tract cancers, brain cancers, cancers of reproductive organs, digestive tract cancers, urinary tract cancers, eye cancers, liver cancers, skin cancers, head and neck cancers, thyroid cancers, parathyroid cancers, and metastases of any of the foregoing.

20. The method of any one of claims 4-18, wherein the disease is chosen from lymphomas, sarcomas, melanomas, glioblastomas, and leukemias.

21. The method of any one of claims 4 to 20, further comprising administering to the subject a therapeutically effective amount of at least one immune checkpoint inhibitor.

22. The method of claim 21, wherein the immune checkpoint inhibitor is chosen from PD-1 inhibitors, PD-L1 inhibitors, and CTLA-4 blockers.
